SEQUENCE LISTING (1 sequence)

SEQ ID NO: 1 - protein, 8 aa, artificial sequence
  eight amino acid sequence tag that binds to streptavidin
WSHPQFEK

(19) United States
(12) Patent Application Publication
(10) Pub. No.: US 2019/0062706 A1
(43) Pub. Date: Feb. 28, 2019

(54) SELECTIVE EXPANSION OF DIFFERENT SUBPOPULATIONS OF T CELLS BY THE ALTERATION OF CELL SURFACING SIGNALS AND SIGNAL RATIO

(71) Applicant: LIFE TECHNOLOGIES AS, Oslo (NO)

(72) Inventors: Hilde ALMAASBAK, Oslo (NO); Tanja AARVAK, Oslo (NO); Kerstin BERNSTROEM, Oslo (NO); Oystein AAMELLEM, Jar (NO)

(21) Appl. No.: 15/771,821

(22) PCT Filed: Oct. 27, 2016
(86) PCT No.: PCT/EP2016/075973

§ 371 Date: Apr. 27, 2018

(30) Foreign Application Priority Data
Oct. 28, 2015 (GB) 1519059.8
Aug. 30, 2016 (GB) 1614644.1

(51) Int. Cl. C12N 5/0783

(52) U.S. Cl. CPC C12N 5/0637; C12N 2501/599; C12N 2501/51; C12N 2501/515

(57) ABSTRACT

This invention relates, inter alia, to compositions of expanded T cell populations, methods for the expansion of T cell populations and methods for using such populations of cells. In some aspects, the invention relates to compositions and methods for the selective expansion of T cell subpopulations present in mixed T cell populations, as well as T cell subpopulations produced by methods for the invention.

### FIELD OF THE INVENTION

This invention relates, inter alia, to compositions of expanded T cell populations, methods for the expansion of T cell populations and methods for using such populations of cells. In some aspects, the invention relates to compositions and methods for the selective expansion of T cell subpopulations present in mixed T cell populations, as well as T cell subpopulations produced by methods for the invention.

### BACKGROUND OF THE INVENTION

The ability of T cells to recognize the universe of antigens associated with, for example, various cancers or infectious organisms is conferred by T cell antigen receptor (TCR), which is made of both an α (alpha) chain and a β (beta) chain or a γ (gamma) and a δ (delta) chain. The proteins which make up these chains are encoded by DNA, which employs a unique mechanism for generating the tremendous diversity of the TCR. This multi-subunit immune recognition receptor associates with the CD3 complex and binds to peptides presented by the major histocompatibility complex (MHC) class I and II proteins on the surface of antigen-presenting cells (APCs). Binding of TCR to the antigenic peptide on the APC is the central event in T cell activation, which occurs at an immunological synapse at the point of contact between the T cell and the APC. To sustain T cell activation, T lymphocytes typically require a second co-stimulatory signal. Co-stimulation is typically necessary for a T helper cell to produce sufficient cytokine levels that induce clonal expansion. Utilizing exogenously administered cytokines and stimulators of cell surface proteins T cells can be expanded and activated ex vivo.

Ex vivo expanded T cells are widely used in translational research and in clinical trials for the immunotherapy of cancer and opportunistic infections, and to prevent autoimmunity and graft versus host disease (GVHD) after transplantation. Although the administration of ex vivo expanded T cells has produced some positive clinical results, the complexity of the manufacturing of large number of various T cell products remains a major limitation for a broader application. While early ex vivo culture conditions only aimed at producing high T-cell numbers and resulted in cells expressing unfavorable phenotypes with low in vivo proliferative capacity, focus has been shifted towards protocols yielding T cells with better in vivo engraftment, proliferation and functionality.

Some of these earlier protocols utilized DYNABEADS® CD3/CD28 CTS™ in the activation and expansion of T cells. This method can produce populations of T cells that display a less differentiated phenotype and demonstrate increased anti-tumor activity and superior persistence compared to T cells expanded with soluble anti-CD3 antibody (OKT-3) and IL-2 in various in vivo models. Similar protocols using a combination of CD3 and CD28 binding and/or signaling can be used for the isolation and activation of polyclonal T cells. However, they do not provide optimal activation of antigen experienced memory T cells, such as tumor infiltrating lymphocytes (TILs) and virus-specific T cells.

The quality and quantity of the primary T-cell activation signal, and the presence and type of costimulatory ligands, cytokines and growth factors are believed to determine the overall signal delivered to the T cells and ultimately influence on the fate of the activated T cell. The successful expansion of such antigen experienced T cells depends on adequate strength of stimulation via TCR/CD3, as well as on the provision of optimal co-stimulatory signals and cytokines. Some reports show that using relatively strong CD3 signaling capability predominately expands naïve T cells and are believed to delete antigen experienced T cells due to activation-induced cell death (AICD) (Kalamasz et al., Immunother 27:405 (2004)). Antigen experienced memory T cells and various subsets thereof have broad ranging therapeutic implications in the treatment of cancers, autoimmune disorders, inflammatory diseases, allergic diseases, and infectious diseases. Therefore, there is a long felt need for reliable, efficient and rapid way to expand specific immune subpopulations, such as antigen experienced memory T cells, regulatory T cells (Tregs), and Th17 cells.

The present invention addresses this need for subpopulation specific expansion of specific T cell subtypes from the general T cell population and provides additional benefits as well.

### SUMMARY OF THE INVENTION

The present invention provides, inter alia, compositions and methods for the selective expansion of various T cell subpopulations. These T cell subpopulations include, but are not limited to: (1) CD4+CD25+FOXP3+ regulatory T cells (Treg), a suppressive subset of CD4+ T helper cells important for the regulation of immune responses; (2) Th17 cells, an inflammatory subset of CD4+ T helper cells that regulate host defense, and are involved in tissue inflammation and various autoimmune diseases; and (3) memory T cells, or antigen specific T cells, a long lasting cell type that retains immunity to prior exposed antigens. Disclosed herein are methods and compositions for the selective expansion of each of the above described T cell subpopulations. The present invention provides the T cell subpopulation in sufficient numbers for therapeutic uses as well (e.g., adoptive immunotherapy, in vivo infusions, etc.). The methods and compositions described herein can be used to generate different T cell subpopulations for research purposes and/or for clinical use. The resultant expanded T cell subpopulations have wide ranging uses in clinical settings.

In some aspects the invention relates to compositions and methods for selectively expanding members of T cell subpopulations. Such methods include those that comprise exposing a mixed population of T cells to (a) a first agent that provides a primary activation signal to the members of the T cell subpopulation, thereby activating the T cells, and (b) a second agent and a third agent, each of which stimulates two or more different accessory molecules on the members of the T cell subpopulation, thereby stimulating the proliferation of the activated T cells of (a). In some aspects, the ratios of the first agent, the second agent, and the third agent may be adjusted to induce the members the T cell subpopulation to selectively expand over members of other T cell subpopulations. The first agent may be an antibody (e.g., an anti-CD3 antibody). The second agent may also be an antibody. The third agent may be an antibody, a non-antibody protein, or a chemical agent (e.g., rapamycin). When a non-antibody protein is used, this protein may be a chemokine or cytokine (e.g., Interleukin-1α, Interleukin-2, Interleukin-4, Interleukin-1β, Interleukin-6, Interleukin-12, Interleukin-15, Interleukin-18, Interleukin-21, Transforming growth factor β1, etc.).

In some specific aspects, the ratio of the first agent, the second agent, and the third agent may be adjusted such that the first agent is lower in concentration compared to the second and/or third agent. Further, the lower concentration of the first agent may be about 0.06 units, about 0.1 units, about 0.2 units, about 0.3 units, about 0.34 units, or about 0.4 units. Also, the first agent may be an anti-CD3 antibody at a concentration of about 0.34 units, and the second agent may be an anti-CD28 antibody at a concentration of 3.4 units.

In some aspect, anti-CD3 may be used in and amounts of 0.01 to 4.0 (e.g., from about 0.01 to about 3.5, from about 0.01 to about 3.4, from about 0.02 to about 3.4, from about 0.05 to about 3.5, from about 0.1 to about 3.4, from about 0.2 to about 3.4, from about 0.5 to about 3.0, from about 0.1 to about 2.5, from about 0.01 to about 1.0, from about 0.05 to about 1.0, etc.) units and anti-CD28 may be used in and amounts of 1.0 to 5.0 (e.g., from about 1.0 to about 4.8, from about 1.0 to about 4.5, from about 1.0 to about 3.5, from about 1.0 to about 3.0, from about 1.0 to about 2.9, from about 1.5 to about 3.5, from about 2.0 to about 3.5, from about 2.5 to about 3.5, from about 1.0 to about 4.8, etc.) units. Further, the ratio of anti-CD3 to anti-CD28 may be in the range of from 1:10 to 1:50 (e.g., from about 1:12 to about 1:48, from about 1:10 to about 1:48, from about 1:12 to about 1:50, from about 1:10 to about 1:40, from about 1:10 to about 1:35, from about 1:10 to about 1:30, etc.).

In some instances, the T cell subpopulation selectively expanded may be Treg cells. In such instances (as well as other instances), the lower concentration of the first agent may be about 0.01 units. Further, the first agent may be an anti-CD3 antibody at a concentration of about 0.01 units, about 0.06 units, about 0.1 units, about 0.2 units, about 0.3 units, about 0.34 units, or about 0.4 units, and a second agent may be an anti-CD28 antibody, and a third agent may be an anti-CD137 antibody.

In some instances, the T cell subpopulation selectively expanded may be memory T cells. In such instances (as well as other instances), the lower concentration of the first agent may be about 0.005, about 0.01, or about 0.06 units. In other instances, the T cell subpopulation selectively expanded may be Th17 cells. Further, the first agent may be an anti-CD3 antibody at a concentration of about 0.01, about 0.05, about 0.06, about 0.1, or about 0.34 units, and a second agent may be an anti-ICOS antibody.

The invention also includes compositions and methods for selectively expanding T cell subpopulations. Such methods include those that comprise (a) exposing T cells to CD3, CD28 and/or CD137 signals ex vivo, and (b) culturing said T cells in a manner that allows for the expansion of Th17 cells, antigen experienced T cells and/or regulatory T cells. In some instances, the CD3, CD28 and CD137 signals may be mediated by anti-CD3, anti-CD28 and/or anti-CD137 antibodies. In more specific instances, anti-CD3, anti-CD28 and anti-CD137 antibodies may be used in a range that encompasses the concentrations of 0.01 units to 1.5 units (e.g., for the expansion of memory T cells). Further, anti-CD3, anti-CDS, anti-ICOS, anti-CD6, anti-CD28 and anti-CD137 antibodies may be used in a range that encompasses the concentrations of 0.06 units to 1.5 units (e.g., for the expansion of Th17 cells). Also, anti-CD3, anti-CDS, anti-ICOS, anti-CD6, anti-CD28 and anti-CD137 antibodies may be used in a range that encompasses the concentrations of about 0.34 units to 3.41 units (e.g., for the expansion of Treg cells). Additionally, anti-CD3 antibodies may be used in a lower concentration compared to the concentration of anti-CD5, anti-ICOS, anti-CD6, anti-CD28 and anti-CD137 antibodies. In some aspect, the T cells may be isolated using CD3 selection. Further, Th17 cells expanded by methods of the invention may be CD3⁺, CD8/CD4+/ and may produce IL-17 cytokine. Such Th17 cells may also be capable of producing IL-17, IL-21 and/or IL-22.

Memory T cells expanded by methods of the invention include those selected from the group consisting of stem memory T cells, central memory T cells, and effector memory T cells. Stem memory cells may have one or more of following markers: CD3+, CD45RO−, CCR7+, CD45RA+, CD62L+ (L-selectin), CD27+, CD28+, IL-7Rα+, IL-2Rβ, CXCR3, and LFA-1. Central memory cells may have one or more of following markers: CD3+, CCR7+, CD45RA−, CD45RO+, CD62L+ (L-selectin), CD27+, and CD28+. Further, these central memory cells may be capable of producing IL-2. Effector memory cells expanded by methods of the invention may have one or more of following markers: CD28+/−, CD27+/−, CD3+, CD4+, CD8+, CCR7−, CD45RA−, CD45RO+. Effector memory cells may also be cells capable of producing IFNγ and IL-4. Treg cells expanded by methods of the invention may have one or more of following markers: CD4⁺, CD25+, FOXP3⁺ and CD127^{neg/low}.

The invention further includes compositions and methods for selectively expanding T regulatory cells. These methods include those comprising (a) exposing T cells to CD3 and CD28 signals ex vivo, and (b) culturing the T cells in a manner that allows for the expansion of T regulatory cells. Such CD3 and CD28 signals may be mediated by anti-CD3, and anti-CD28 antibodies. Further, anti-CD3, and anti-CD28 antibodies may each be used in ranges that encompasses concentration range of the 0.34 to 3.4 units. Additionally, anti-CD3 antibodies may be used in a lower concentration compared to the concentration of anti-CD28 and/or antibodies.

The invention also includes compositions and methods for selectively expanding Th17 cells. Such method include those comprising (a) exposing CD3+ T cells to CD3, CD28, CD5 and/or ICOS signals ex vivo, and (b) culturing said CD3+ T cells in a manner that allows for the expansion of Th17 cells, wherein the amount of CD3, CD28, CD5 and/or ICOS may be the same or different. In such methods, the CD3, CD28 and ICOS signals may be mediated by anti-CD3, anti-CD28, anti-CD5, and anti-ICOS antibodies. Further, the anti-CD3, anti-CD28, anti-CD5, and anti-ICOS antibodies may be used in a range that encompasses the concentration range of about 0.06 to about 1.5 units for the expansion of Th17 cells. Additionally, the anti-CD3 antibodies may be used in a lower concentration compared to the concentration of anti-CD28 and/or anti-ICOS antibodies.

The invention additionally includes compositions and methods for selectively expanding antigen experienced T cells. These methods include those comprising (a) exposing T cells to CD3, CD28, CD27 and/or CD137 signals ex vivo, and (b) culturing said T cells in a manner that allows for the expansion of antigen experienced T cells. The CD3, CD28 and CD27 and/or anti-CD137 signals may be provided by anti-CD3, anti-CD28, anti-CD27 and/or anti-CD137 antibodies. The anti-CD3, anti-CD28, anti-CD27 and anti-CD137 antibodies may be used in a range that encompasses the concentration range 0.01-1.5 units. The anti-CD3 antibodies may be used in a lower concentration compared to the concentration of anti-CD28 and/or anti-CD137 antibodies.

The invention also includes compositions comprising CD3+ (1) T cells and (2) beads containing (a) anti-CD3 antibodies and (b) anti-CD28, anti-CD137, anti-ICOS, or anti-CD5 antibodies capable of selective expansion of T cell subpopulations. In some instances, the amount of (a) anti-CD3 antibodies and (b) anti-CD28, anti-CD137, anti-ICOS, or anti-CD5 antibodies present may be the same or different. In particular instances, the T cell subpopulation may be selected from the group consisting of Th17 cells, antigen experienced T cells and/or regulatory T cells. Further, the anti-CD3, anti-CD28, anti-CD5, anti-ICOS, anti-CD27 and anti-CD137 antibodies may be used in a range that encompasses the concentration range of 0.01 to 1.5 units (e.g., for the expansion of memory T cells), in a range that encompasses the concentration range of 0.06 to 1.5 units (e.g., for the expansion of Th17 cells), or in a range that encompasses the concentration range of 0.34 to 3.41 units (e.g., for the expansion of Treg cells). In some instances, the anti-CD3 antibodies may be used in a lower concentration compared to the concentration of anti-CD28 and anti-CD137 antibodies.

The invention further includes compositions comprising (1) T cells and (2) beads containing anti-CD3, anti-CD28, anti-ICOS, anti-CD5, and/or anti-CD137 antibodies capable of selective expansion of Th17 cells, wherein the Th17 cells may be capable of producing one or more effector cytokines. In some instances, the amounts of anti-CD3 and anti-CD28 antibodies present on the beads may be the same or different. Further, the one or more effector cytokine may be one or more cytokine selected from the group consisting of IL-17, IL-21, and IL-22.

The invention includes composition comprising (1) T cells and (2) beads containing anti-CD3, anti-CD28, and anti-CD137 antibodies capable of selective expansion of antigen experienced memory T cells. In some instances, the T cells may be capable of recognizing specific antigen and, wherein the amount of anti-CD3, anti-CD28 and anti-CD137 antibodies present on the beads may be the same or different. The specific antigen may be selected from the group consisting of viral antigens (e.g., CMV, EBV, Influenza, HIV, etc.), bacterial (e.g., Streptococci M-protein, Neisseria pilli, Borrelia burgdorferi lipoprotein VisE, B. pseudomallei polysaccharide antigens etc.,), fungal or protozoal (e.g., Aspergillus fumigatus galactomannan, or F. tularensis lipopolysaccharide, etc.), and cancer antigens.

The invention also includes compositions comprising (1) T cells and (2) beads containing anti-CD3 and anti-CD28 antibodies that are capable of selectively expanding regulatory T cells (e.g., regulatory T cells that are CD4+ CD25+ FOXP3+ CD127^{low/neg}). In some instances, the amount of anti-CD3 and anti-CD28 antibodies present on the beads may be the same or different. The regulatory T cells activity may comprise suppressive activity.

The invention also includes compositions and methods for (a) treating an individual in need thereof, (b) reconstituting an immune system of an individual in need thereof, and (c) providing adoptive immunotherapy to an individual in need thereof. Such methods comprise administering to the individual a pharmaceutically acceptable composition comprising Th17 cells, antigen experienced T cells, and/or regulatory T cells. Individuals in need thereof may be affected by cancer, inflammatory diseases, autoimmune diseases, allergic disease, or infectious diseases, transplant related disease. Further, exemplary cancers include lung, ovarian, pancreatic, breast, liver and skin cancer Inflammatory disease may be selected from the group consisting of diabetes; rheumatoid arthritis; inflammatory bowel disease; familial mediterranean fever; neonatal onset multisystem inflammatory disease; tumor necrosis factor (TNF) receptor-associated periodic syndrom (TRAPS); deficiency of interleukin-1 receptor antagonist (DIRA); Systemic Lupus; Uveitis; and Behcet's disease.

In such methods (as well as other methods of the invention), the T cells may be genetically modified. Further, the genetic modification may be result in the presence of one or more chimeric antigen receptor or a genetically modified T cell receptor.

The invention also includes compositions and methods for selectively altering the proportional ratio of two T cell subtypes in a sample. Such methods include those comprising contacting a sample comprising a mixed population of T cells with at least two stimulatory agents. In some instances, the stimulatory agents provide different amounts of signals to the T cells in the mixed population. In specific instances, one T cell subtype may selectively expand as compared to a second T cell subtype. Further, the sample may comprise buffy coat cells derived from an individual, as well as a sub-set of buffy coat cells (e.g., mononuclear cells). Further, the at least two stimulatory signals may stimulate CD3 and CD28 receptors. Also, at least one T cell subtype may be selectively eliminated from the mixed population. In some aspects, Treg T cells may be increased in proportion with respect to all T cells within the mixed population. One method of doing this is through contacting of the cells with rapamycin in an amount suitable for eliminating non-Treg cells from the population. In additional aspects, the total number of memory T cells may be decreased in the sample. Further, the amount of stimulatory signal CD3 may be less than half than the CD28 stimulatory signal.

The invention also includes methods for expanding Th17 cells through the stimulation of CD3 and CD5 cell surface receptors. In some embodiments, the invention includes method for expanding Th17 cells, these methods may comprise: (a) exposing a population of T cells to CD3 and CD5 signals ex vivo and (b) culturing the population of T cells under conditions that allows for the expansion of Th17 cells. In specific embodiments of the invention, the population of T cells may be exposed to one or more aryl hydrocarbon receptor agonist (e.g., 6-formylindolo[3,2-b]carbazole (FICZ)) and/or may not be exposed to exogenous Interleukin-23. In some instances, the population of T cells is a mixed population of different T cells types. Further, method of the invention include those comprising contacting the population of T cells with one or more polarizing agents (e.g., Interleukin-1β, Interleukin-23, Tumor Growth Factor-β, Interleukin-6, Interleukin-21, Interleukin-2, anti-Interleukin-4 antibody, and/or anti-Interferon γ antibody).

The term “exogenous”, when used in reference to a protein, refers to a protein that is present in a composition but is not produced by a cell present in the composition. For example, a T cell present in a sample may produce IL-2. In such an instance, exogenously added IL-2 refers to IL-2 that is put into the sample as a non-cellular composition (e.g., IL-2 in a buffer).

In some embodiments, the Th17 cells are engineered to express one or more chimeric antigen receptors. These at least one of the one or more chimeric antigen receptors include those that have specificity for a cell surface antigen of a mammalian cell (e.g., an antigen associated).

The invention also includes compositions comprising a CD3 signal and a CD5 signal, In some instances, compositions of the invention may contain one or more aryl hydrocarbon receptor agonist (e.g., 6-formylindolo[3,2-b]carbazole (FICZ)), one or more cytokine (e.g., Interleukin-1β, Interleukin-23, Tumor Growth Factor-β, Interleukin-6, Interleukin-21 and/or Interleukin-2) and/or one or more antibody (e.g., anti-Interleukin-4 antibody, and/or anti-Interferon γ antibody). In specific instances, compositions of the invention may contain Interleukin-10 and Interleukin-6. Compositions such as those above may further comprise a population of T cells. Further, the CD3 signal in compositions of the invention may be one or more anti-CD3 antibody. Also, the CD5 signal in compositions of the invention may be one or more anti-CD5 antibody.

Compositions of the invention may also comprise a population of T cells, a CD3 signal, a CD5 signal, an aryl hydrocarbon receptor agonist, and one or more cytokine. In specific embodiments, compositions of the invention may comprise the population of T cells is present in a mixture comprising: (a) a “buffy coat” sample, (b) a sample of white blood cells that contains greater than 80% mixed T cells, (c) a sample that contains greater than 80% CD4+ T cells, or (d) a sample that contains greater than 80% Th17 cells.

The invention further relates to methods for the separation and activation of T cells from mixed populations of cells (T cells and non-T cells, such as B cells). In some embodiments, such methods comprise: (a) contacting the mixed population of cells with one or more solid supports having bound thereto at least a first ligand with binding affinity for a protein located on T cells present in the mixed population of cells, under conditions that allow for binding of the T cells to the solid support and activation of the same T cells, and (b) separation of the T cells bound to the solid support from cells not bound to the solid support to obtain a purified T cell population. The one or more solid support may have bound thereto at least a first ligand and/or at least a second ligand, wherein each of the first ligand and the second ligand have binding affinity for different proteins located on individual T cells present in the mixed population of cells. Further, the first ligand and the second ligand may be bound to the same solid support or different solid supports. In some instances, the first ligand may be either an anti-CD3 antibody or an anti-CD4 antibody. Further, the second ligand may be any number of ligands, including a ligand selected from the group consisting of: (a) an anti-CD5 antibody, (b) an anti-CD28 antibody, (c) an anti-CD137 antibody, and (d) an anti-ICOS antibody. Further, methods of the invention include those comprising releasing cells of the purified T cell population from solid supports, including the release of T cells obtained in step (b) from the solid support. Additionally, methods of the invention include methods comprising expanding the released T cells once released from solid supports. In many instances, expansion of the released T cells will occur in a culture medium (e.g., a culture medium wherein one or more chemokine or cytokine is present). Further, one or more chemokine or cytokine may be present in step (a). Such one or more chemokine or cytokine include those selected from the group consisting of: (a) Interleukin-1α, (b) Interleukin-2, (c) Interleukin-4, (d) Interleukin-1β, (e) Interleukin-6, (f) Interleukin-12, (g) Interleukin-15, (h) Interleukin-18, (i) Interleukin-21, and (j) Transforming growth factor β1.

Methods of the invention also include methods for the activation and expansion of T cells. Such methods include those comprising contacting mixed populations of T cells with: (a) a first agent which provides a primary activation signal to the members of a T cell subpopulation (e.g., Treg cells, Th17 cells, etc.) by stimulating a molecule on the members of the T cell subpopulation, and (b) a second agent that stimulates a molecule on the members of the T cell subpopulation that is different than the molecule stimulated by the first agent. In many instances, T cells in the population (e.g., members of the T cell subpopulation) are activated and expand. In additional instances, the first agent and the second agent may be bound to one or more solid supports. Further, the T cells may be maintained under conditions that allow for expansion (e.g., expansion of members of the T cell subpopulation). Additionally, the solid supports may be removed from contact with the T cells after a time period of less than 120 hours. In other words, the solid supports are placed in contact with the T cells for a limited time period, then removed from contact with the T cells. This time period may be from about 12 hours to about 120 hours (e.g., from about 12 hours to about 120 hours, from about 24 hours to about 120 hours, from about 36 hours to about 120 hours, from about 48 hours to about 120 hours, from about 60 hours to about 120 hours, from about 48 hours to about 96 hours, from about 60 hours to about 96 hours, from about 72 hours to about 96 hours, etc.). As discussed elsewhere herein, the first agent may an anti-CD3 antibody. Further, the second agent may also be an antibody. Additionally, the T cells may be contacted with a third agent is an antibody or a non-antibody protein (e.g., a chemokine or a cytokine).

Each of the aspects and embodiments described herein are capable of being used together, unless excluded either explicitly or clearly from the context of the embodiment or aspect.

### BRIEF DESCRIPTION OF THE DRAWINGS

FIGS. 1A and 1B are line plots showing the fold expansion of CD4+CD25+CD127low/− flow cytometry sorted Tregs and retention of FOXP3+ cells (˜90% FOXP3+ cells) following activation with DYNABEADS® Treg Expander CD3/CD28. FIG. 1A is a line plot showing fold expansion following activation with DYNABEADS® Treg Expander CD3/CD28. The data indicate efficient expansion of several hundred fold. FIG. 1B is a line plot showing % FOXP3+ cell retention. The data indicate the cells retain high FOXP3 expression after 14 days in expansion culture. Cells are re-stimulated using the same prototype bead at day 9. Increased expansion is achieved with DYNABEADS® conjugated with a lower CD3 amount.

FIG. 2 is a bar graph showing the fold expansion of CMV specific memory T cells at day 10 post-activation. Fold expansion negatively correlates with increased signal strength provided by DYNABEADS® Memory Cell Expanders conjugated with increasing amounts of the agonistic CD3 antibody.

FIG. 3A through FIG. 3D are graphs showing IL-17 expression in T cells cultured with Th17-polarizing conditions and expanded with DYNABEADS® Th17 Expander CD3/ICOS (anti-CD3 antibody conjugated at 1.5 and 0.06; and two different ICOS clones) or DYNABEADS® CD3/CD28 (Cell Therapy System). Starting on day 3, IL-2 is added to the cultures. At day 10-13 cultures are stimulated with PMA-ionomycin for 4-5 hours before assessment of the of IL-17 expression. Histograms show intracellular expression of IL-17. FIG. 3A is a graph showing IL-17 expression from cells expanded with DYNABEADS® Th17 Expander CD3/ICOS (ISA-3), CD3 high (1.5). FIG. 3B is a graph showing IL-17 expression from cells expanded with DYNABEADS® Th17 Expander CD3/ICOS (ISA-3), CD3 low (0.06). FIG. 3C is a graph showing IL-17 expression from cells expanded with DYNABEADS® Th17 Expander CD3/ICOS (ICOS clone C398.4A purchased from eBiocienses, Affymetrix), low CD3 (0.06). FIG. 3D is a graph showing IL-17 expression from cells expanded with DYNABEADS® CD3/CD28 CTS™. The activation signal strength and the nature of co-stimulation highly influence polarization and expansion of Th17 cells. Note: activation with DYNABEADS® Th17 Expander CD3/ICOS (mid-0.3) result in a similar phenotype as “(high-1.5)” at a bead:cell ratio of 1:1.

FIG. 4A through FIG. 4C are graphs showing T cells from 3 donors activated with DYNABEADS® Th17 Expanders CD3/ICOS. FIG. 4A is a series of line graphs of fold T cell expansion from donors A, B, and C respectively. FIG. 4B is a series of bar graphs indicating the percentage of IL-17 producing cells resultant from selective expansion of T cells from donors A, B and C respectively with DYNABEADS® Th17 Expanders CD3/ICOS with differing bead:cell ratios (reported as BC). FIG. 4C is a series of bar graphs indicating the relative number of IL-17 producing cells resultant from expanding T cells from donors A, B, and C respectively with differing bead:cell ratios (reported as BC).

FIG. 5. Effect of neutralizing antibodies. Average % IL-17 producing CD4 T cells (n=3 donors) stimulated with CD3/ICOS beads or CD3/CD28 beads, and polarized with (+) or without (−) neutralizing antibodies. P-values (paired T test) given for variations caused by blocking antibodies (NSD, not statistically different (top panel). Individual results; single donors (A,B,C) and ICOS versus CD28 stimulation (bottom panels).

FIG. 6. Effect of costimulation on cytokine production. Purified CD3+ T cells activated with Dynabeads prototypes CD3/CD28, CD3/CD5, or CD3/ICOS expands a Th17 subset with variable efficacy where typically CD3/CD5 being the most efficient, followed by CD3/ICOS and with CD3/CD28 being the less efficient stimulatory prototype. CD3/CD5 stimulation typically induces IL-17A production in 25-50% of the CD4+ T cells, with a high proportion being polyfunctional IL-17+INF-γ+ upon restimulation day 10-13.

FIG. 7. Effect of costimulation on phenotype. T cells stimulated by CD3/CD28, CD3/CD5 or CD3/ICOS co-express various levels of CCR4 and CCR6 post-expansion, with CD3/CD5 and CD3/ICOS stimulation generating a higher fraction of CCR4+CCR6+ cells (77 and 73%) compared to CD3/CD28 (46%).

FIG. 8. T cells were activated with CD3/CD5 (upper) or CD3/ICOS (lower) figure and expanded in presence of standard Th17-polarizing cytokines/antibodies (cytokines package), or with 100 nM FICZ and IL-1β and IL-6. At day 13 cultures were stimulated with PMA/Ionomycin for 4-5 hours before assessment of IL-17A and IL-17F expression. Cytograms show intracellular expression of the cytokines.

FIG. 9A. Early removal of stimulatory DYNABEADS® improves TH17 polarization. T cells were activated with CD3/CD5, CD3/ICOS, or CD3/CD28 beads in presence of standard Th17-polarizing cytokines/antibodies (TGF-β, IL-23, IL-6, IL-1β, and αIL-4/α-IFN-γ neutralizing antibodies). Stimulatory DYNABEADS® were i) kept in culture throughout expansion, ii) removed day 2 (48 h) post-activation, or iii) removed day 3 (72 h) post-activation. At day 13 cultures were stimulated with PMA/Ionomycin for 4-5 hours before assessment of intracellular IL-17A and IL-17F expression in CD4+ T cells.

FIG. 9B shows fraction and absolute number of CD4+ T cells expressing the Th17 associated surface marker CD161 day 10 post-activation of cells prepared as set out in the FIG. 9A legend.

FIG. 10A. CD3/CD5 isolated T cells can be directly polarized and expanded. T cells were isolated from PBMC using DYNABEADS® CD3/CD5 at a bead: cell ratio of 1:3, 1:1 and 3:1. The isolation efficiency was compared to using DYNABEADS® CD3/CD28 CTS, which is commonly used to enrich for T cells in T cell stimulation protocols. Isolation efficiency (%) is shown.

FIG. 10B. The isolated T cells of FIG. 10A were simultaneously expanded in presence of standard Th17-polarizing cytokines/antibodies (TGF-β, IL-23, IL-6, IL-1β, and αIL-4/α-IFN-γ neutralizing antibodies). Stimulatory DYNABEADS® were removed day 3 post-activation. At day 13 cultures were stimulated with PMA/Ionomycin for 4-5 hours before assessment of intracellular IL-17A and IL-17F expression in CD4+ T cells.

### DETAILED DESCRIPTION

In some aspects, the present invention provides, inter alia, methods and compositions for the selective expansion of specific T cell subpopulations. These T cell subpopulations include, but are not limited to, Th17 cells, regulatory T cells (Tregs) and memory T cells. The specific T cell subpopulations disclosed herein can be used for the treatment of various physiological conditions, diseases, and/or disease states.

### Some Aspects of the Invention

In some aspects, the invention is based upon signal types and signal intensities for the activation and/or expansion of T cell subpopulations. Along these lines, it has been observed that specific T cell subpopulations may be obtained and/or enhanced in a mixed population by selective cell surface marker stimulation. It has further been observed that variations in T cell receptor signal strength can also be used to obtain specific T cell subpopulations. In many instances, T cell subpopulations will be obtained from a mixed T cell population (e.g., total T cells obtained from peripheral blood).

Stimulation of T cell receptors can have a number of effects on a particular T cell, for example (1) no effect upon the T cell, (2) T cell activation, (3) T cell proliferation, (4) T cell polarization, (5) T cell differentiation (e.g., memory T cells), and (6) the induction of apoptosis in the T cell. The effect generated will often be a function of factors, such as the specific T cells present, the nature of the stimulatory signal(s), the ratio of the strength of multiple stimulatory signals (e.g., two, three, four, etc. signals) when multiple signals are employed, and the total or individual signal strength to which the T cell is exposed.

In many instances, T cells will be separated from other cell types prior to receptor stimulation. This may be done in a single step or in multiple steps. Exemplary methods are as follows: (1) buffy coat or apheresis isolation of mononuclear cells, (2) isolation of CD4+ cells using, for example, magnetic beads having one or more CD4 receptor binding agent, and (3) fluorescence activated cell sorting (see Example 1).

In some aspects of the invention, the ratio of two or more T cell signals are adjusted in a manner that results in selective expansion of a first set of one or more T cell subpopulations over a second set of one or more T cell subpopulations. In many instances, the first set of one or more (e.g., one, two, three, four, five, etc.) T cell subpopulations will be smaller than the second set of one or more T cell subpopulations. In some instances, the first set of one or more T cell subpopulations may comprise a single T cell subpopulation and the second set of one or more T cell subpopulations may comprise all of the other T cell subpopulations present. In some instances, a first T cell subpopulation (e.g., antigen experienced (memory) T cells) will be selectively expanded over a second T cell subpopulation (e.g., naive T cells). Further, one or more additional T cell subpopulations may expand in conjunction with the first T cell population.

In many instances, one signal will be generated by stimulation of a first T cell receptor (e.g., the CD3 receptor) and another signal will be generated by stimulation of a second, co-stimulation T cell receptor (e.g., the CD28 receptor, the CD137 receptor, the CD27 receptor, the CD5 receptor, the CD6 receptor, the ICOS receptor, the CD134 receptor, etc.). Signal ratios may be altered in manner that (a) enhances the expansion of a particular T cell population, (b) enhances the elimination of another T cell population (e.g., via apoptosis, inhibition of cell growth, by having no expansion effect, etc.), or both (a) and (b). In some instances, one or more additional T cell receptors may also be stimulated or other signals may be provided to the T cells.

Exemplary ratios of stimulation signal of a first T cell receptor to stimulation signal of a second T cell receptor will vary with the T cell subpopulation that is sought to be obtained and may be from about 50:1 to about 1:200 (e.g., about 1:5, about 1:10, about 1:15, about 1:20, about 1:40, from about 50:1 to about 1:40, from about 50:1 to about 1:30, from about 40:1 to about 1:40, from about 30:1 to about 1:40, from about 40:1 to about 1:20, from about 40:1 to about 1:10, from about 50:1 to about 1:1, from about 50:1 to about 5:1, from about 40:1 to about 5:1, from about 50:1 to about 10:1, from about 50:1 to about 15:1, from about 50:1 to about 20:1, from about 40:1 to about 5:1, from about 30:1 to about 3:1, from about 20:1 to about 3:1, from about 15:1 to about 3:1, from about 10:1 to about 5:1, from about 1:5 to about 1:10, from about 1:3 to about 1:20, from about 1:8 to about 1:25, from about 1:3 to about 1:40, from about 1:5 to about 1:50, from about 1:10 to about 1:50, from about 1:10 to about 1:100, from about 1:10 to about 1:150, from about 1:10 to about 1:200, from about 1:5 to about 1:150, from about 1:5 to about 1:200, etc.).

For purposes of illustration, signal provided by anti-CD3 antibodies and anti-CD28 antibodies may be present in a ratio of 1:10. It has been found that for expansion of some T cell subpopulations a lower amount of CD3 signal is desirable over a second signal (e.g., a CD28 signal and/or a CD137 signal). In some instances, when more than two T cell receptor signals are provided the ratio of each signal may be different or two or more of the signal ratios may be the same (e.g., two of three). As an example, CD3, CD28, and CD137 signals may be present at a ratio of 1:10:10. When each of these signals are generated by antibodies, this will generally mean that one part of an anti-CD3 antibody is present with ten parts of both anti-CD28 and anti-CD137 antibodies. This, of course, assumes that the amount of receptor stimulation is equal for each of the three receptors by their cognate antibody.

One issue for consideration is characterization of mixtures containing population of T cells generated by methods of the invention. In many instances, compositions and methods for the invention will be directed to altering the ratio of T cells of particular subpopulations in a mixture. For example, methods of the invention may result in certain types of T cells being eliminated from a mixed population by, as examples, apoptosis or dilution. Thus, one aspect of the invention relates to the amount of enhancement or depletion of a T cell population in a mixture, as well as the mixtures themselves. For example, if there are two T cell subpopulations in a mixture (e.g., Th17 T cells and Th1 T cells) and these subpopulations are present in, for example, a 1:1 ratio, then the invention includes methods in which one T cell subpopulation is increased in proportion to the other T cell subpopulation. For purposes of illustration the ratio may be altered to from about 1:1.5 to about 1:100,000 (e.g., from about 1:1.5 to about 1:100,000, from about 1:1.5 to about 1:80,000, from about 1:1.5 to about 1:50,000, from about 1:1.5 to about 1:10,000, from about 1:1.5 to about 1:5,000, from about 1:2,500 to about 1:25,000, from about 1:2,500 to about 1:60,000, from about 1:2,500 to about 1:80,000, from about 1:2,500 to about 1:100,000, from about 1:5,000 to about 1:100,000, from about 1:5,000 to about 1:80,000, from about 1:5,000 to about 1:50,000, from about 1:5,000 to about 1:25,000, etc.).

Further, the invention relates to compositions and methods for altering the ratio of T cells of particular subpopulations in a mixture, where the proportion of one T cell subpopulation is increased over another T cell subpopulation by at least 200,000 fold (e.g., from about 1,000 fold to about 200,000 fold, from about 5,000 fold to about 200,000 fold, from about 10,000 fold to about 200,000 fold, from about 20,000 fold to about 200,000 fold, from about 50,000 fold to about 200,000 fold, from about 75,000 fold to about 200,000 fold, from about 1,000 fold to about 120,000 fold, from about 5,000 fold to about 120,000 fold, from about 10,000 fold to about 120,000 fold, from about 1,000 fold to about 80,000 fold, from about 10,000 fold to about 80,000 fold, etc. An example of what is meant by “fold” is illustrated as follows. If two T cell subpopulations are present in an initial ratio of 1:2, then an alteration in their ratio to 1:8 is a 2 fold increase of one T cell subpopulation with respect to the other T cell subpopulation.

Another factor that can result is the selective expansion of individual T cell subpopulations is stimulus signal strength. By “stimulus signal strength” refers to the total signal strength on a per T cell basis. This includes the strength of the various signals (e.g., a signal stimulating a first T cell surface receptor, a signal stimulation of a second T cell surface receptor, a signal stimulation of a third T cell surface receptor, etc.) and the combined signal to which each T cell in the population is exposed to. Thus, the invention also relates to the amount of stimulatory signal received by each cell in a mixture of various T cell subpopulations. The stimulatory signal can be modulated by alterations to concentrations of stimulatory agents, ratios thereof, or ratios of surfaces comprising said stimulatory agents to cell count. In embodiments where stimulatory agents are bead-based a bead:cell ratio may be altered. A bead:cell ratio may comprise about 1:5000, about 1:2500, about 1:1000, about 1:500, about 1:250, about 1:100, about 1:90, about 1:80, about 1:70, about 1:60, about 1:50, about 1:40, about 1:30, about 1:10, about 1:9, about 1:8, about 1:7, about 1:6, about 1:5, about 1:4, about 1:3, about 1:2, about 1:1, about 2:1, about 3:1, about 4:1, about 5:1, or about 8:1, about 10:1, or about 15:1 beads per cell.

In some instances, one or more cytokine may be added to a T cell population. In many instances, IL-1 beta, IL-2, IL-4, IL-5, IL-6, IL-7, IL-10, IL-12, IL-13, IL-15, IL-21, IL-23, IFN-gamma, and TGF-beta. When Th17 polarization is desired, one or more of the following cytokines may be used: IL-1 beta, IL-6, TGF-β, IL-21, IL-23, and neutralizing anti-IL-4 and anti-IFN-gamma antibodies. Furthermore, IL-1β, IL-6, TGF-β, IL-23, and neutralizing antibodies against IL-4 and IFNγ signals may be used for the selective expansion of Th17 cells.

Table 1 shows a number of different T cell subtypes that may be obtained using methods of the invention. Table 1 shows various signals that may be used to selectively expand T cells of particular types.

TABLE 1
T Cell Subtype Stimuli

| | Signal 1 | Signal 2 | |
|---|---|---|---|
| T cell Subtype | (Agonists) | (Agonists) | Signal 3* |
| Naive T cells | CD3 | CD28 | IL-2 |
| Central Memory | CD3 | CD28 | IL-2, IL-7, |
| T cells | | | IL-15 |
| Effector Memory | CD3 | CD137 and/or | IL-2, IL-15, |
| T cells | | CD27 and/or CD28 | IL-7, IL-21 |
| | | and/or other | |
| Treg | CD3 | CD5, CD28, and/or | IL-2 |
| | | CD278 (ICOS) | |
| Th17 | CD3 | CD5, CD6, and/or | IL-21, TGFβ, |
| | | CD278 (ICOS) | IL-6, IL-1beta |
| Th2 | CD3 | CD28 | IL-4 |
| Th1 | CD3 | CD28 | IL-2, IL-12 |
| Th22 | CD3 | CD28 and/or | IL-23, IL-6 |
| | | other | |
| Th9 | CD3 | CD28 and/or | IL-4, TGFβ |
| | | other | |

*“and/or” in each instance where more than an agent is set out.

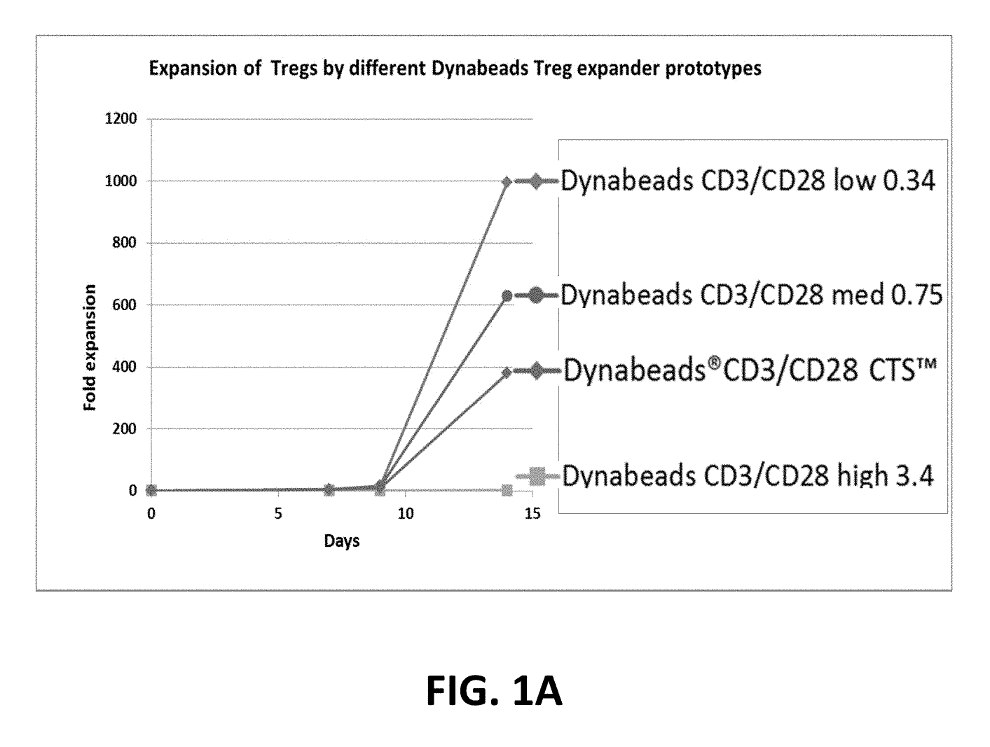

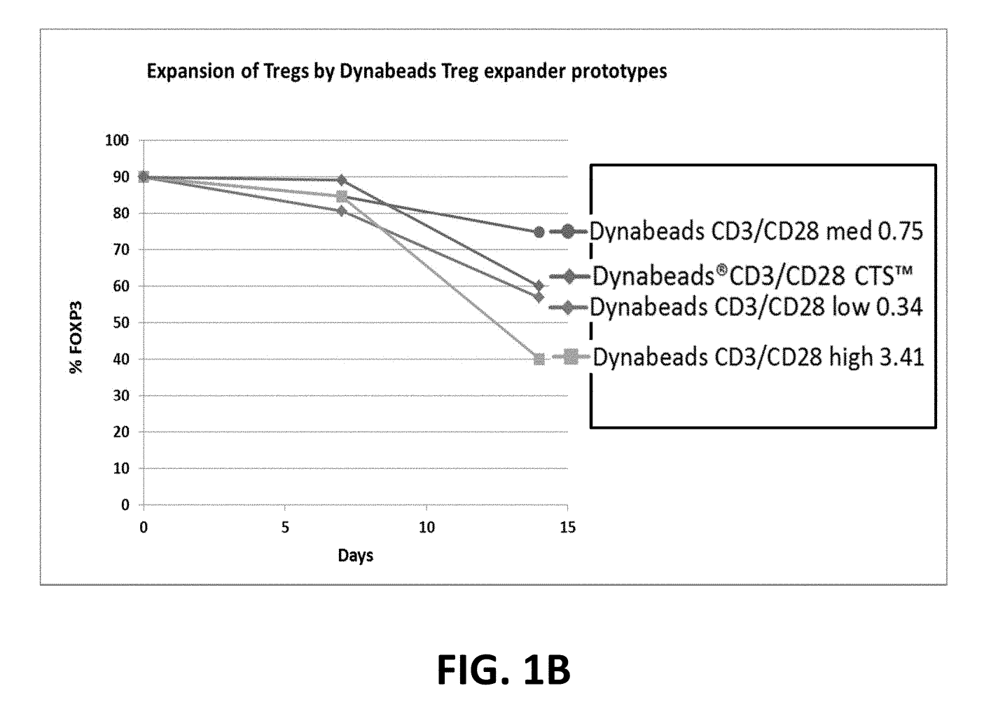

In particular, the invention includes methods for the selective expansion of one or more T cell subpopulations. Such methods result in the enhancement or depletion of specific T cells in a sample. As an example, naïve T cells, memory T cells, Th1 T cells and regulatory T cells (Tregs) stimulation of CD3 and CD28 receptors in conjunction with Interleukin-2. It has been shown that naïve T cells may be expanded while memory T cells may be depleted from a sample by the adjustment of total CD3/CD28 stimulus (see U.S. Pat. No. 8,617,884, the disclosure of which is incorporated herein by reference). It has now been shown, related to one aspect of the invention, that different T cell subpopulations may be selectively expanded by adjusting signal ratios and total signal strength. As an example, Treg cells expand well when CD3 signal is lower than CD28 signal (see FIG. 1A). The identification of selective expansion conditions can be used to increase the proportion of members of one T cell subpopulation over member of one or more other T cell subpopulations in a sample, even when the various cells of the various T cell subpopulations expand in response to the same stimuli. For purposes of illustration, assume that Treg T cells represent 1% of a mixed population and naïve T cells, memory T cells are represent, respectively, 1.5%, 3% of the same mixed population, stimulatory signals may be adjusted to induce elimination of memory T cells, while selectively expanding Treg T cells. The net result may be a mix population where Treg T cells represent 40% and naïve T cells, memory T cells, and Th1 T cells are represent, respectively, 2%, 0.5% and 2.5% of the mixed population.

An additional agent that may be used for the selective enhancement or depletion of one or more T cell subtypes (e.g., CD4+CD25+FOXP3+ regulatory T cells, CD4+CD25+FOXP3− regulatory T cells, CD4+CD25− T cells, etc.) is rapamycin.

### Mammalian Immune System

The mammalian immune system uses two general adaptive mechanisms to protect the body against environmental pathogens. When a pathogen-derived molecule is encountered, the immune response is highly activated to ensure protection against that pathogenic organism.

The first mechanism is the non-specific (or innate) inflammatory response. The innate immune system can recognize specific molecules that are present on pathogens but not on the body itself. The second mechanism is the specific or acquired (or adaptive) immune response. Adaptive immune responses are custom tailored to the pathogen in question. The adaptive immune system evolves a specific immunoglobulin (antibody) response to many different molecules present in the pathogen, called antigens. In addition, a large repertoire of T cell receptors is sampled for their ability to bind processed forms of the antigens bound to MHC class I and II on antigen-presenting cells (APCs), such as dendritic cells (DCs).

The immune system recognizes and responds to structural differences between self and non-self proteins. Proteins that the immune system recognizes as non-self are referred to as antigens. Pathogens typically express large numbers of highly complex antigens. Acquired immunity has specific memory for antigenic structures; repeated exposure to the same antigen increases the response, which increases the level of induced protection against that particular pathogen.

Acquired immunity is mediated by specialized immune cells called B and T lymphocytes (or simply B and T cells). B cells produce and mediate their functions through the actions of antibodies. B cell-dependent immune responses are referred to as “humoral immunity,” because antibodies are detected in body fluids. T cell-dependent immune responses are referred to as “cell mediated immunity,” because effector activities are mediated directly by the local actions of effector T cells. The local actions of effector T cells are amplified through synergistic interactions between T cells and secondary effector cells, such as activated macrophages. The result is that the pathogen is killed and prevented from causing diseases.

Immune cells can require specific stimulation for activation. The use of anti-CD3/CD28, for example, provides the activation signal for some T cell population. Naïve T cells are believed to require at least two signals for activation. Signal one is antigen specific and is elicited by peptide/major histocompatibility complex (MHC) complexes presented by antigen-presenting cells (APC) and received through the T-cell receptor (TCR)/CD3 complex. For some T cell subpopulations, signal two can be delivered by antigen presenting cells and one of the candidate molecules for its receptor is the T cell antigen CD28. It is thought that when both the TCR/CD3 and CD28 T cell receptors are occupied by appropriate ligands, T cells are stimulated to proliferate and produce IL-2 (a cytokine essential for T cell proliferation), whereas occupation of the T cell receptor alone favors T cell anergy or apoptosis.

In vitro it has been shown that T cell growth and cytokine production can be stimulated by culturing T cells with anti-CD3 antibodies which have been immobilized to a solid phase (for example beads or tissue culture plates) and adding soluble CD28 antibodies (Sommer et al., Eur. J. Immunol. 23:2498-2502 (1993), Sunder-Plassmann et al., Blood 87:5179-5184 (1996)). More recently it has been shown that co-immobilizing both CD3 and CD28 antibodies to the same solid phase or to different solid phases can also induce T cell proliferation (Levine et al., J. of Immunol. 159:592130 (1997); Li et al., Science 283:848-851 (1999)).

The present invention allows for one of skill to produce various T cell subpopulations in a reliable, effective and efficient manner as well as use the expanded T cell subpopulations for different purposes, including but not limited to, therapeutic purposes and research/discovery purposes. As further described below, the T cell subpopulations include, but are not limited to, regulatory T cells, Th17 cells and antigen experienced memory cells.

### Regulatory T Cells

Aspects of the present invention relate to methods for efficiently generating regulatory T cells (or “T regulatory cell” or “Treg”) and the use of these methods in the generation of T cell populations which have applications in, for example, immunotherapy. Treg cells can be characterized by markers, such as CD4+, CD25+, FOXP3+, CD127^{neg/low}. In some instances, Treg cell expanded using compositions and methods of the invention will be CD4+, CD25+, FOXP3−. Compositions and methods for generating FOXP3− regulatory T cells are set out in Aarvak et al., U.S. Pat. No. 9,119,807.

Naturally occurring regulatory T (Treg) cells negatively regulate the activation of other T cells, including effector T cells, as well as innate immune system cells and can be utilized in immunotherapy against autoimmune diseases and provide transplantation tolerance. Various populations of Treg cells have been described and include naturally occurring CD4+CD25+FOXP3+ cells and induced Tr1 and Th3 cells that secrete IL-10 and TGF respectively.

Treg cells are characterized by sustained suppression of effector T cell responses. Traditional or conventional Treg cells can be found, e.g., in the spleen or the lymph node or in the circulation. Tregs are proven highly effective in preventing GVHD and autoimmunity in murine models. Clinical trials with adoptive transfer of Tregs in transplantation, treatment of diabetes and other indications are underway. The relative frequency of Tregs in peripheral blood is approximately 1-2% of total lymphocytes implicating the necessity of ex vivo expansion of Tregs prior to adoptive transfer for most clinical applications. Producing sufficient Tregs during the ex vivo expansion has been a major challenge in applying Treg therapy to humans.

### Th17 Cells

T helper 17 cells (or “Th17 cells” or “Th17 helper cells”) are an inflammatory subset of CD4+ T helper cells that regulate host defense, and are involved in tissue inflammation and various autoimmune diseases. Th17 cells have been found in various human tumors however their function in cancer immunity is unclear. When adoptively transferred into tumor-bearing mice, Th17 cells have been found to be more potent at eradicating melanoma than Th1 or non-polarized (Th0) T cells (Muranski et al., Blood. 112:362-373 (2008)). Th17 cells are developmentally distinct from Th1 and Th2 lineages. Th17 cells are CD4+ cells that are responsive to IL-1R1 and IL-23R signaling and produce the cytokines IL-17A, IL-17F, IL-17AF, IL-21, IL-22, IL-26 (human), GM-CSF, MIP-3α, and TNFα. The phenotype of Th17 cells is CD3+, CD4+, CD161+. One obstacle to the use of Th17 cells for adoptive cell transfer has been the identification of robust culture conditions that can expand the Th17 cell subset, as well as their unstable phenotype in vivo (tumor microenvironment).

The invention relates, in part, to compositions and methods for the generation of specific T cell subtypes. One specific T cell subtype that may be produced using compositions and methods of the invention are Th17 cells. Thus, in some aspects, the invention relates to compositions and methods for the expansion (e.g., the selective expansion) of Th17 cells using CD3 signaling (e.g., via anti-CD3 antibodies) and CD5 signaling (e.g., via anti-CD5 antibodies), one or more cytokine and neutralizing antibodies (e.g., anti-IL-4 antibodies and anti-IFNγ antibodies). In some instances, compositions and methods for the selective expansion of Th17 cells are adjusted in a manner that results in a decrease in the amount of or the elimination of one or more neutralizing antibody.

One issue with many T cells is their plasticity. In other words, some T cells can change to become different subtypes. Also, this T cell variation is often mediated by T cells in the surrounding environment. For example, a number of T cell subtypes express IL-4 and/or IFNγ. These proteins have physiological effects on surrounding T cells and, in some instances, mediate differentiation of one type of T cell into another type of T cell.

The expansion (e.g., selective expansion) of T cell subsets, such as Th17 cells, may be performed by the use of one or more signal, adjusting the intensity of one or more signal, adjusting the ratio of intensity between two or more signals, and the elimination of one or more signal. Conditions may be selected based upon ratios of two parameters or more than two parameters. Two examples are as follows: (1) Ratios of CD3 signal and CD5 signal may be adjusted and (2) ratios of CD3 signal and CD5 signal may be adjusted may be combined with adjustment of the total CD3 signal and CD5 signal. By way of example, when it is desirable to expand Th17 cells, two signals may be chosen for use (e.g., CD3 signal and CD5 signal). These signals may be adjusted, for example, in ratio of from about 1:1 to about 1:50 (e.g., from about 1:1 to about 1:50, from about 1:1 to about 1:30, from about 1:1 to about 1:20, from about 1:1 to about 1:15, from about 1:5 to about 1:15, from about 1:5 to about 1:20, from about 1:8 to about 1:12, from about 1:8 to about 1:15, from about 1:8 to about 1:20, etc.).

The invention also includes the use of aryl hydrocarbon receptor (AHR) antagonists for the expansion of T cells (e.g., Th17 T cells). It has been found that Th17 T cells may be expanded by exposure of T cells to CD3 signal (e.g., agonistic anti-CD3 antibody), CD5 signal (e.g., agonistic anti-CD5 antibody), IL-1β, IL-6, and an AHR agonist (e.g., FICZ).

Exemplary AHR ligands that may be used in the practice of the invention include, but are not limited to, FICZ (6-formylindolo[3,2-b]carbazole), dFICZ (6,12-diformylindolo[3,2-b]carbazole), 2,3,7,8-tetrachlorodibenzo-p-dioxin (TCDD), 2-(1′H-indole-3′-carbonyl)-thiazole-4-carboxylic acid methyl ester (ITE), quercetin, indol-3-carbinol, resveratrol, curcumin, M50367 {3-[2-(2-phenylethyl) benzoimidazole-4-yl]-3-hydroxypropanoic acid, and VAF347 {[4-(3-chloro-phenyl)-pyrimidin-2-yl]}, tryptophan derivatives, such as indigo dye and indirubin, tetrapyrroles such as bilirubin, and the arachidonic acid metabolites lipoxin A4 and prostaglandin G.

In some instances, the invention includes compositions and methods for the expansion of Th17 cells, wherein T cells are exposed to the following reagents: one or more agonistic anti-CD3 antibody, one or more agonistic anti-CD5 antibody, and one or more AHR agonist (e.g., FICZ). In some instances, one or more cytokine may also be used. Exemplary cytokines include IL-1β and IL-6. Thus, in some instances, T cells are exposed to one or more agonistic anti-CD5 antibody, one or more AHR agonist (e.g., FICZ), IL-1β and IL-6.

In some instances, the invention includes compositions and methods for the expansion of Th17 cells, wherein T cells are exposed to the following reagents: one or more agonistic anti-CD3 antibody, one or more agonistic anti-CD5 and/or anti-CD28 antibody, and one or more cytokine. Exemplary cytokines include TGF-β, IL-1β, IL-6, and IL-23. Thus, in some instances, T cells are exposed to one or more agonistic anti-CD3 antibody, one or more agonistic anti-CD5 and/or anti-CD28 antibody, TGF-β, IL-1β, IL-6, and IL-23.

The invention thus includes compositions and methods for the expansion of Th17 cells, wherein cells of a starting T cell population is not exposed to TGF-β and/or IL-23.

T cell populations used in the practice of the invention may reside, as examples, in (i) a “buffy coat” sample, (ii) a sample of white blood cells depleted of non-T cells (e.g., a sample that contains greater than 80% mixed T cells), (iii) a sample of CD4+ T cells (e.g., a sample that contains greater than 80% CD4+ T cells), or a sample that contains predominantly Th17 cells (e.g., a sample that contains greater than 80% Th17 cells). Thus, in some instances, methods of the invention related to the selective expansion of one or more T cell sub-population.

With respect to specific formulations for the expansions of Th17 T cells, as well as associated expansion methods, conditions will typically be adjusted to achieve a high level of Th17 cell expansion.

One factor that may be adjusted is the ratio of CD3 signal (e.g., agonistic anti-CD3 antibody) to CD5 signal (e.g., agonistic anti-CD5 antibody). In many instances, the amount of CD3 signal will be lower than the CD5 signal. Exemplary ratios of CD3 to CD5 signal include ratios of from about 1 to about 1.5, from about 1 to about 2, from about 1 to about 3, from about 1 to about 5, from about 1 to about 8, from about 1 to about 10, from about 1 to about 12, from about 1 to about 15, from about 1 to about 20, from about 1 to about 25, from about 1 to about 30, from about 1 to about 40, from about 1 to about 50, from about 1 to about 75, from about 1 to about 100, etc.

Exemplary antibodies that may be used in the practice of the invention include the BC3 anti-CD3 antibody, a clone which expresses this antibody being available from the American Type Culture Collection (HB-10166), and UCHT2 anti-CD5 antibody, available from a number of sources, including eBioscience, Inc., San Diego, Calif.

Antibodies in general tend to vary in specificity and affinity for the cognate ligands. Thus, ratios of CD3 and CD5 signal may be adjusted to be equivalent to ratios of BC3 antibody and UCHT2 antibody. In other words, the ratios referred to above relate, in part, to data derived from BC3 antibody and UCHT2 antibody, with the ratios of these antibodies inducing a physiological effect on T cells. Thus, other CD3 and CD5 signals may be used and the amounts of these other signals may be adjusted to achieve the same physiological effect. Put another way, the invention includes various CD3 and CD5 signaling agents that achieve the same physiological effect of the ratios of BC3 antibody and UCHT2 antibody set out above. Such physiological effects may be measured by methods set out elsewhere herein (e.g., Example 10, and data associated therewith).

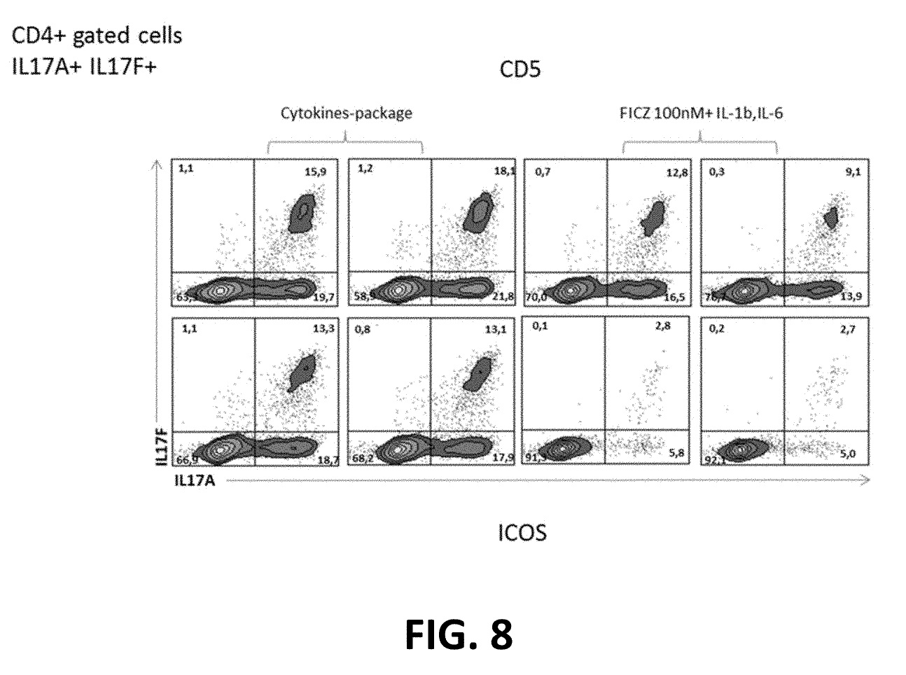

Reagents in addition to CD3 and CD5 signaling agents may be present in compositions of the invention and may be used in methods of the invention. Some of these additional reagents include AHR agonist (e.g., FICZ), cytokines (e.g., IL-1β and IL-6), and one of more neutralizing antibody (e.g., anti-αIL-4 and anti-α-TN-γ neutralizing antibodies). Reagents may be adjusted to achieve, for example, results shown in FIG. 8. In particular, the amounts of AHR agonist and cytokines employed will typically be in the range of from about 1nM to about 1 mM (e.g., from about 1 nM to about 800 nM, from about 1 nM to about 500 nM, from about 1 nM to about 250 nM, from about 25 nM to about 1 mM, from about 25 nM to about 500 nM, from about 25 nM to about 300 nM, from about 50 nM to about 300 nM, from about 75 nM to about 500 nM, from about 75 nM to about 300 nM, etc.).

### Memory T Cells

Memory T cells, or antigen-experienced cells, are experienced in a prior encounter with an antigen. These T cells are long-lived and can recognize antigens and quickly and strongly affect an immune response to an antigen to which they have been previously exposed. Memory T cells can encompass: stem memory cells (T_{SCM}), central memory cells (T_{CM}), effector memory cells (TEM). T_{SCM} cells have the phenotype CD45RO−, CCR7+, CD45RA+, CD62L+ (L-selectin), CD27+, CD28+ and IL-7Ra+, but they also express large amounts of IL-2Rβ, CXCR3, and LFA-1. T_{CM} cells express L-selectin and the CCR7, they secrete IL-2, but not IFNγ or IL-4. T_{EM} cells do not express L-selectin or CCR7 but produce effector cytokines like IFNγ and IL-4.

The present invention provides methods and compositions for the selective expansion of the above T cell subpopulations. Prior methodologies do not allow one of skill in the art to selectively expand for specific T cell subtype in a manner described herein. While the signal strength to some extent can be regulated by varying the ratio of standard DYNABEADS® CD3/CD28 to T cells (Kalamazs et al., J. Immunother. 27:405-418 (2004)), the local concentration of stimulatory CD3-antibodies at the contact area between the DYNABEADS® and the cell surface is unaffected and still high in this approach. Data herein disclose the amounts and ratios of primary and various costimulatory antibodies conjugated to the surface of DYNABEADS® that allow for a fine-tuning of the T-cell response resulting in selective activation and expansion of the various specific T cell subsets as Th17, antigen experienced T cells, and Tregs.

### I. Definitions

Unless defined otherwise herein, all technical and scientific terms used herein have the same meaning as commonly understood by one of ordinary skill in the art to which this invention pertains.

Antibodies for use in methods of the present invention may be of any species, class or subtype providing that such antibodies can react with the target of interest, e.g., CD3, the TCR, or CD28 as appropriate.

Thus “antibodies” for use in the present invention include:

(a) any of the various classes or sub-classes of immunoglobulin (e.g., IgG, IgA, IgM, IgD or IgE derived from any animal e.g., any of the animals conventionally used, e.g., sheep, rabbits, goats, mice, camelids, or egg yolk),

(b) monoclonal or polyclonal antibodies,

(c) intact antibodies or fragments of antibodies, monoclonal or polyclonal, the fragments being those which contain the binding region of the antibody, e.g., fragments devoid of the Fc portion (e.g., Fab, Fab′, F(ab′)2, scFv, V_{H}H fragments, or other single domain antibodies), the so called “half molecule” fragments obtained by reductive cleavage of the disulphide bonds connecting the heavy chain components in the intact antibody. Fv may be defined as a fragment containing the variable region of the light chain and the variable region of the heavy chain expressed as two chains.

(d) antibodies produced or modified by recombinant DNA or other synthetic techniques, including monoclonal antibodies, fragments of antibodies, “humanized antibodies”, chimeric antibodies, or synthetically made or altered antibody-like structures.

Also included are functional derivatives or “equivalents” of antibodies, e.g., single chain antibodies, CDR-grafted antibodies, etc. A single chain antibody (SCA) may be defined as a genetically engineered molecule containing the variable region of the light chain, the variable region of the heavy chain, linked by a suitable polypeptide linker as a fused single chain molecule. Also included are VHH antibodies that may be monovalent or bivalent.

Methods of preparation of antibody fragments and synthetic and derivatized antibodies are well known in the art and widely described in the literature and are not be described herein.

The term “activation,” as used herein, refers to the state of a cell following sufficient cell surface moiety ligation to induce a measurable morphological, phenotypic, and/or functional change. Within the context of T cells, such activation may be the state of a T cell that has been sufficiently stimulated to induce cellular proliferation. Activation of a T cell may also induce cytokine production and/or secretion, and up- or down-regulation of expression of cell surface molecules such as receptors or adhesion molecules, or up- or down-regulation of secretion of certain molecules, and performance of regulatory or cytolytic effector functions. Within the context of other cells, this term infers either up- or down-regulation of a particular physico-chemical process.

The term “unit,” as used herein with respect to antibodies, is tied to anti-CD3 mediated physiological effect. In particular, 0.34 units of anti-CD3 is the amount of antibody that will result in a 1000 fold expansion of Treg cells after 14 days, when contacted with a mixed T cell population in the present of 3.4 units of anti-CD28 (see Example 2 and FIG. 1). Units are defined by the number of antibody molecules present. Thus, in the above instance, there are ten times more anti-CD28 molecules than anti-CD3 molecules.

The term “stimulation,” as used herein, refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event, such as binding the TCR/CD3 complex. Further, the stimulation event may activate a cell and up- or down-regulate expression of cell surface molecules such as receptors or adhesion molecules, or up- or down-regulate secretion of a molecule, such as down-regulation of Tumor Growth Factor beta (TGF-β). Thus, ligation of cell surface moieties, even in the absence of a direct signal transduction event, may result in the reorganization of cytoskeletal structures, or in the coalescing of cell surface moieties, each of which could serve to enhance, modify, or alter subsequent cell responses.

The term “agent”, “ligand”, or “stimulatory agent”, as used herein, refers to a molecule that binds to one or more defined population of cells (e.g., members of T cell subpopulations) and induces a cellular response. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), or the like. Within the specification and in the context of T cell stimulation, antibodies are used as a prototypical example of such an agent.

The terms “selective expansion” and “selectively expanding” as used herein in reference to T cells, refer to the ability of certain T cells to expand under condition where other T cells either will not expand or will expand at a lower rate. As an example, assume T cell subtypes 1 and 2 are present in a mixed population in respective percentages of 5% and 10% of the total T cells present. If certain conditions result in T cell subtype 1 representing 30% and T cell subtype 2 representing 12% of the total T cells present, then T cell subtype 1 is selectively expanded over T cell subtype 2, even though T cell subtype 2 is now a larger portion of the total T cell population. T cell subtype 2 is selectively expanded over general members of the mixed population of T cells in the sense that, as a total percentage of T cell, T cell subtype 2 became present with an increased “frequency”. Thus, selective expansion relates to the expansion of a particular T cell subtype over the general population of T cells and will often result T cell subtypes also expanding. The above example can be referred to as conditions for the selective expansion of T cell subtype 1, even though T cell subtype 2 also expands.

The term “exposing” as used herein, refers to bringing into the state or condition of immediate proximity or direct contact.

The term “proliferation” as used herein, means to grow or multiply by producing new cells.

A “subject” can be a vertebrate, a mammal, or a human. Mammals include, but are not limited to, farm animals, sport animals, pets, primates, mice and rats. In one aspect, a subject is a human. A “subject” can be a “patient” (e.g., under the care of a physician) but in some cases, a subject is not a patient.

A “co-stimulatory signal,” as used herein, refers to a signal, which in combination with a primary signal, such as TCR/CD3 ligation, leads to T cell proliferation and/or activation and/or polarization.

“Separation,” as used herein, includes any means of substantially purifying one component from another (e.g., by filtration, affinity, buoyant density, or magnetic attraction).

A “surface,” as used herein, refers to any surface capable of having an agent attached thereto and includes, without limitation, metals, glass, plastics, co-polymers, colloids, lipids, cell surfaces, and the like. Essentially any surface that is capable of retaining an agent bound or attached thereto.

As used herein, the singular terms “a,” “an,” and “the” include the plural reference unless the context clearly indicates otherwise.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

### II. Methods of Producing T Cell Subpopulations

Methods of the invention can be utilized to selectively expand one or more substantially pure specific T cell subpopulation(s). Exemplary T cell subpopulations include, but are not limited to, Treg cells, Th17 cells and memory T cells. Example uses for the expanded T cell subpopulations are disclosed herein.

### Sources of Mixed Population of T Cells

The starting source for a mixed population of T cell can be blood (e.g., circulating blood) which may be isolated from a subject. Circulating blood can be obtained from one or more units of blood or from an apheresis or leukapheresis. The apheresis product typically contains lymphocytes, including T cells, monocytes, granulocytes, B cells, other nucleated white blood cells, red blood cells, and platelets. T cells can be obtained from a number of sources, including blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources, including cord blood. T cells may also be obtained from a xenogeneic source, for example, from mouse, rat, non-human primate, and pig.

In certain embodiments of the present invention, T cells can be obtained from a unit of blood collected from a subject using any number of techniques known to the skilled artisan, such as Ficoll separation. T cells may be isolated from the circulating blood of a subject. Blood may be obtained from the subject by apheresis or leukapheresis. The apheresis product typically contains lymphocytes, including T cells, monocytes, granulocytes, B cells, other nucleated white blood cells, red blood cells, and platelets. Prior to exposure to a sensitizing composition and subsequent activation and/or stimulation, a source of T cells is obtained from a subject. In some embodiments, the cells collected by apheresis may be washed to remove the plasma fraction and to place the cells in an appropriate buffer or media for subsequent processing steps. In some embodiments of the invention, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, Baxter) according to the manufacturer's instructions. After washing, the cells may be resuspended in a variety of biocompatible buffers, such as, for example, calcium (Ca)-free, magnesium (Mg)-free PBS. Alternatively, the undesirable components of the apheresis sample may be removed and the cells directly resuspended in culture media.

In other embodiments, T cells are isolated from peripheral blood lymphocytes by lysing or removing the red blood cells and depleting the monocytes, for example, by centrifugation through a PERCOLL™ gradient. A specific subpopulation of T cells, can be further isolated by positive or negative selection techniques.

In some embodiments, T cells can be positively selected for CD3+ cells. Any selection technique known to one of skill in the art may be used. One non-limiting example is flow cytometric sorting. In another embodiment, T cells can be isolated by incubation with a solid support to which anti-CD3 antibody is bound. One non-limiting example is anti-CD3/anti-CD28-conjugated beads, such as DYNABEADS® Human T-Expander CD3/CD28 (Life Technologies Corp., Cat. No. 11141D), for a time period sufficient for positive selection of the desired T cells. In a further embodiment, the time periods ranges from 30 minutes to 36 hours or longer and all integer values there between. In a further embodiment, the time period is at least 1, 2, 3, 4, 5, or 6 hours. In another embodiment the time period is 10 to 24 hours. In one embodiment, the incubation time period is 24 hours. Longer incubation times, such as 24 hours, can increase cell yield. Longer incubation times may be used to isolate T cells in any situation where there are few T cells as compared to other cell types. In one aspect of the present invention, enrichment of a T cell population by negative selection can be accomplished with a combination of antibodies directed to surface markers unique to the negatively selected cells. One possible method is cell sorting and/or selection via magnetic immunoadherence or flow cytometry that uses a cocktail of monoclonal antibodies direct to cell surface markers present on the cells negatively selected. In some embodiments, the fold expansion may differ based on the starting materials due to the variability of donor cells. In some embodiments, the normal starting density can be between about 0.5×10⁶-1.5×10⁶.

Additionally, T cell subpopulations may be generated by selection on the basis of whether one or more marker(s) is/are present or absent. For example, Treg cells may be obtained from a mixed population based upon the selection of cells that are CD4⁺, CD25+, CD127^{neg/low} and, optionally, FOXP3+. In some instances, Treg cells may be FOXP3−. Selection, in this instance, effectively refers to “choosing” of the cells based upon one or more definable characteristic. Further, selection can be positive or negative in that it can be for cells have one or more characteristic (positive) or for cells that do not have one or more characteristic (negative).

With respect to Treg cells, for purposes of illustration, these cells may be obtained from a mixed population through the binding of these cells to a surface (e.g., magnetic beads) having attached thereto antibodies that bind to CD4 and/or CD25 and the binding of non-Treg cells to a surface (e.g., magnetic beads) having attached thereto antibodies that binding CD127. As a specific example, magnetic beads having bound thereto an antibody that binds to CD3 may be used to isolate CD3+ cells. Once released, CD3+ cells obtained may then be contacted with magnetic beads having bound thereto an antibody that binds to CD4. The resulting CD3+, CD4+ cells may then be contacted with magnetic beads having bound thereto an antibody that binds to CD25. The resulting CD3+, CD4+, CD25+ cells may then be contacted with magnetic beads having bound thereto an antibody that binds to CD127, where the cells that are collected are those that do not bind to the beads.

In some instances, multiple characteristics may be used simultaneously to obtain a T cells subpopulation (e.g., Treg cells). For example, a surface containing bound thereto antibodies that bind to two or more cell surface marker may also be used. As a specific example, CD4+, CD25+ cells may be obtained from a mixed population through the binding of these cells to a surface having attached thereto antibodies that bind to CD4 and CD25. The selection for multiple characteristics simultaneously may result in number of undesired cells types “co-purifying” with the desired cell type(s). This is so because, using the specific example above, cells that are CD4+, CD25− and CD4−, CD25+ may be obtained in addition to CD4+, CD25+ cells.

Flow cytometry is particularly useful for the separation of cells based upon desired characteristics. Cells may be separated based upon detectable labels associated with molecules that bind to cells of interested (e.g., a natural ligand such as IL-7 binding to CD127, an antibody specific for CD25, etc.). Thus, ligands that bind to cellular components that may be detected and/or differentiated by flow cytometry systems may be used to purify/isolate T cells that have specific characteristics. Further, the presence or absence of multiple characteristics may be simultaneously determined by flow cytometry.

The invention thus include methods for obtaining members of one or more T cell subpopulations, where members of the T cell subpopulations are identified by specific characteristics and separated from cells with differ with respect to these characteristics. Examples of characteristics that may be used in methods of the invention include the presence or absence of the following proteins CD3, CD4, CD5, CD8, CD11c, CD14, CD19, CD20, CD25, CD33, CD34, CD45, CD56, CD123, CD127, CD278, CD335, and FOXP3.

Selection of characteristics related to cells that one seek to obtain will vary with cells present in the particular sample. For example, umbilical cord blood (UCB) has been shown to contain Treg cells that can lessen the effects of graft vs. host disease. UCB, however, contains cell ratios not normally found in peripheral blood. One category of cells found in UCB is hematopoietic stem cells (HSCs). HSCs are generally CD34+, which may be separated from CD4+, CD25+ cells by either binding to surfaces that have attached thereto suitable ligands or by labeled molecules that bind to one or more of CD4+, CD25+, and/or CD34+.

The invention thus relates to compositions and method for purifying cells where the number of cells of interest in a mixture are increased at least 5 fold (e.g., from about 5 fold to about 50 fold, from about 10 fold to about 50 fold, from about 15 fold to about 50 fold, from about 20 fold to about 50 fold, from about 25 fold to about 50 fold, from about 10 fold to about 40 fold, from about 10 fold to about 30 fold, etc.). An example of purification is where a cell type represents 5% of the total cells in a mixed population prior to purification and represents 25% of the total cells in a mixed population prior to purification. This is an example of 5 fold purification.

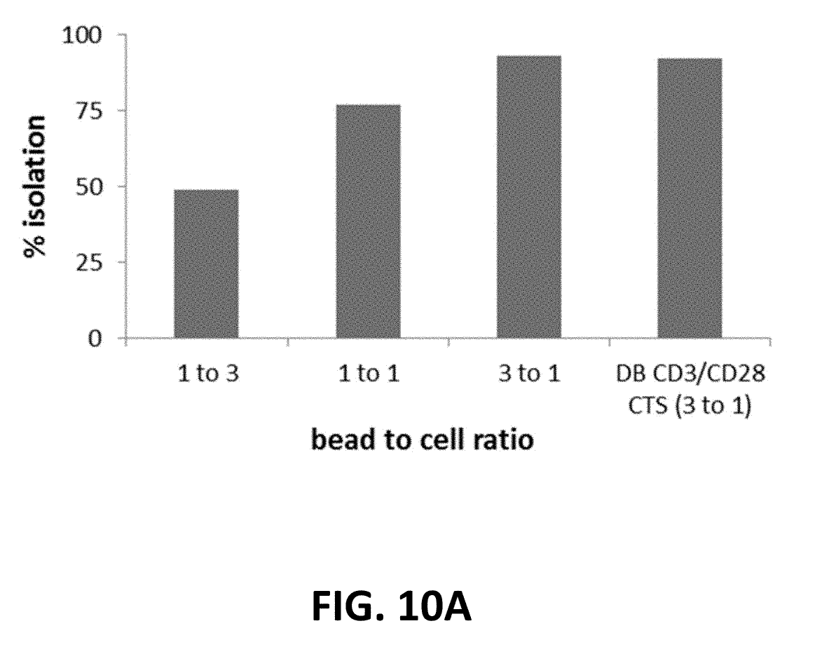

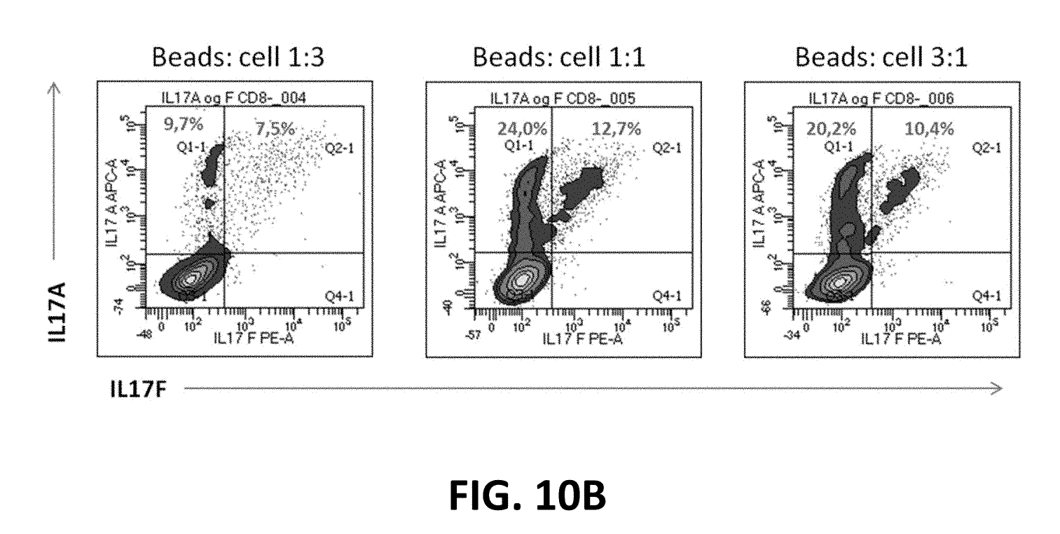

As can be seen in FIG. 10A and FIG. 10B, CD3/CD5 isolated T cells can be directly polarized and expanded. One starting material for Th17 polarization protocols is CD3 or CD4 enriched cells (positive or negative isolation).

Upstream isolation is laborious and costly, and complicates processes for Th17 generation. It has been found that CD3/CD5 beads (same as used for Th17 polarization) efficiently isolate CD3 cells (T cells) with an efficiency similar to using DYNABEADS® CD3/CD28 CTS (>90% at a bead:cell ratio of 3:1, and 80% at a bead:cell ratio of 1:1 ratio) (FIG. 10A). As shown in FIG. 10B, such positively CD3/CD5 isolated T cells can be directly polarized to Th17 cells.

The invention also relates to compositions and methods for the simultaneous activation and purification of T cells. An example of such compositions and methods include the formation of a mixture containing magnetic beads with anti-CD3 and anti-CD5 antibodies bound hereto, where the reaction mixture includes Th17 T cells. In this instance, the Th17 T cells may be both activated by the anti-CD3 and anti-CD5 antibodies and separated from other cells that do not contain CD3 and/or CD5 surface markers. Of course, proteins other than CD3 and CD5, as well as various combinations of proteins (e.g., CD3 and CD28; CD3, CD28, and CD137; CD3 and CD137; CD3 and CD278; etc.) may be used for both the activation and purification of T cells.

Further, it has been found that, when magnetic beads are used to simultaneously activate and purify T cells, the bead to cell ratio can be adjusted to enhance purification efficiency. For example, it was found using DYNABEADS® CD3/CD28 CTS™ (Thermo Fisher, cat. no. 40203D) that a 3:1 bead to cell ratio could be used to obtain a T cell population that was >90% pure and a 1:1 bead to cell ratio could be used to obtain a T cell population that was about 80% pure. Thus, the invention provides compositions and methods for the purification of T cells where the bead to cell ratio is in the from about 20:1 to about 1:5 (e.g., from about 20:1 to about 1:2, from about 20:1 to about 1:1, from about 20:1 to about 2:1, from about 20:1 to about 3:1, from about 10:1 to about 1:1, from about 10:1 to about 2:1, from about 10:1 to about 3:1, etc.). The invention also provides compositions and methods for the purification of T cells where the bead to cell ratio is adjusted to yield an activated T cell population that is at least 80% pure (e.g., from about 80% to about 99%, from about 83% to about 99%, from about 85% to about 99%, from about 88% to about 99%, from about 90% to about 99%, from about 93% to about 99%, from about 95% to about 99%, from about 80% to about 95%, from about 85% to about 95%, etc.).

Purified T cells obtained by the above methods may be mixed in type or may be a specific type (e.g., Treg cells). For example, CD3 and CD28 proteins are present on the surfaces of a number of different T cell sub-types. Thus, T cells purified using anti-CD3 and anti-CD28 antibodies will often be a mixed population. Specific T cell sub-types may be expanded out of such mixed populations using additional agents such as chemokines, cytokines, or other agent (e.g., rapamycin, an aryl hydrocarbon receptor agonist, etc.). Thus, purified, mixed populations of T cell may be used to generate T cells of one or more sub-type by adjustment of activation and/or expansion conditions.

For example, Th17 cells may be purified, activated and expanded by processes such as the following. T cells may be purified from a mixed cell population using either an anti-CD3 antibody or an anti-CD4 antibody bound to a solid support (positive isolation). Cells bound to the solid support are collected and exposed to a solid support containing anti-CD3 antibody and an anti-CD5 antibody. T cells that bind to these beads are separated from unbound cells and then exposed to agents that facilitate the polarization of Th17 cells (e.g., IL-1β, IL-6, an aryl hydrocarbon receptor antagonist, etc.). The T cells are then maintained under conditions that allow for the expansion of Th17 cells. In some instances, other T cells may expand under the conditions used. Conditions will normally be adjusted such that Th17 cells expand more rapidly than other T cell sub-types.

Expansion and/or Proliferation to Various T Cell Subpopulations

Mixed population of T cells isolated from a subject can be expanded into various T cell subpopulations by varying their exposure to a primary activation signal with a primary agent. The primary activation signal is anti-CD3 and can be achieved with a primary agent that is anti-CD3 (e.g., anti-CD3 antibody or other agent with binding specificity for CD3). The primary activation signal can be used in combination with second agent and/or a third agent, which can be directed to CD28, CTLA-4, CD137, CD27, CD5, CD6, CD134, CD2, LFA-1, CD40, SLAM, GITR, and/or ICOS.

The cells of the invention can be expanded by incubation in culture with the agents as described above and herein. The compositions of the invention comprise specific T cell expansion agents in defined ratios conjugated on a surface. T cell expansion agents of the invention may be present with one or more costimulatory signals. Ratios of a T cell expanding agent to a costimulatory signal may be 1:1000, about 1:500, about 1:100, about 1:50, about 1:40, about 1:30, about 1:20, about 1:10, about 1:5, about 1:4, about 1:3, or about 1:2. In some embodiments, two or more costimulatory signals may be present for example at ratios of 1:1000:1000, about 1:500:500, about 1:100:100, about 1:50:50, about 1:40:40, about 1:30:30, about 1:20:20, about 1:10:10, about 1:5:5, about 1:4:4, about 1:3:3, or about 1:2:2. In some embodiments, additional costimulatory signals may be present. In some embodiments, the ratio of the individual costimulatory signals to a primary signal may not be identical (e.g., a ratio of primary signal to costimulatory signals is 1:4:3). Furthermore, a ratio of signal to cell can be controlled by a total amount of signal applied in selective expansion. For example, in a bead-bound stimulatory signal a bead:cell ratio can be altered.

In one embodiment, isolated T cells are expanded by beads comprising surface acting agents. Isolated T cells may be activated and expanded ex vivo by incubation with beads or other compositions of the present invention at a ratio of about 1 bead per cell. In other embodiments, beads may be used in culture to activate and expand T cells at ratio of about 100 cells per bead, a ratio of about 10 cells per bead, a ratio of about 5 cells per bead, a ratio of about 2 cells per bead, a ratio of about 2 beads per cell, a ratio of about 5 beads per cell, a ratio of about 10 beads per cell, or a ratio of about 100 beads per cell.

Bead:cell ratio, total concentrations, and/or relative ratios of costimulatory signals can each be adjusted to achieve maximal fold increase, percentage of desired cell type within the expanded cell population, or relative numbers of the desired cell type. Resultant cell populations may comprise about 10%, about 20%, about 25%, about 30%, about 35%, about 40%, about 45%, about 50%, about 55%, about 60%, about 65%, about 70%, about 75%, about 80%, about 85%, about 90%, about 95%, or about 100% of the desired cell types compared to the total cell population. Relative numbers of desired cell type is defined as the fraction of desired cell multiplied by the total cell population fold expansion. Relative numbers of desired cell type may comprise about 50, about 75, about 100, about 125, about 150, about 175, about 200, about 225, about 250, about 275, about 300, about 350, about 400, or about 500.

For Tregs, the stimulation method may be performed in the presence of other cells, e.g., other T cells, such as CD4+, CD25+ cells which themselves may proliferate to a greater degree during the method of the invention. Thus in the above described protocols, the step of isolating T cells may comprise isolating a significant portion (i.e., at least about 20, about 30, about 40, about 50, about 60, about 70, about 80 or about 90%) or all CD4+ cells, i.e., which include both CD25+ from the sample. Thus in a one embodiment of the invention, the isolation step comprises the isolation of CD4+ cells. Furthermore, other cells may also be present such that Treg, Th17 or memory T cells form only a portion of the cells used in the stimulatory method, before or after the isolation step. Thus in the stimulatory step the CD4+, CD25+ may be present as a portion of the cells subject to stimulation, i.e., an enriched preparation may be used, such as a preparation comprising at least about 50, about 60, about 70, about 80 or about 90% of the total cells subjected to stimulation.

In some instances, at least some CD4−, CD25− cells are absent, e.g., at least about 10%, about 20%, about 30%, about 40%, about 50%, about 60%, about 70%, about 80%, or about 90% of the cells which appeared in the starting material are absent. Alternatively, the starting cells for stimulation/expansion can be substantially all CD4+, CD25+ cells, such as at least about 80%, about 90%, about 95% or about 98% CD4+, CD25+ cells.

Stimulation of T cells to selectively expand Treg cells may include stimulation by an anti-CD3 and an anti-CD28 agent. In contrast to previous anti-CD3/CD28 costimulatory approaches, in some aspects the present invention utilizes a low anti-CD3 to anti-CD28 ratio, such that an anti-CD3 agent is present to a lesser degree than an anti-CD28 agent. The ratio of anti-CD3 to anti-CD28 may be about 1:1000, about 1:500, about 1:100, about 1:50, about 1:40, about 1:30, about 1:20, about 1:10, about 1:5, about 1:4, about 1:3, or about 1:2. In some instances, selectively expand Treg cells may include stimulation by (1) an anti-CD3 and (2) an anti-CD5 and/or an anti-ICOS agent.

Stimulation of T cells to selectively expand Th17 cells may include stimulation agents that innervate with CD3, CD28, CD5 and/or ICOS receptors. In some embodiments, Th17 cells may be produced by using anti-CD3 and anti-ICOS antibodies. In some embodiments of the invention, the level of CD3 receptor stimulation may be in a lower concentration compared to the level of ICOS stimulation. Using antibodies as an example, the ratio of anti-CD3 to anti-ICOS may be about 1:1000, about 1:500, about 1:100, about 1:50, about 1:40, about 1:30, about 1:20, about 1:10, about 1:5, about 1:4, about 1:3, or about 1:2.

Stimulation of T cells to selectively expand T memory cells may include stimulation by an anti-CD3, and anti-CD27 or an anti-CD28 or anti-CD137. In many instances, anti-CD3 agent will be present in a lower concentration compared to anti-CD28, anti-CD27 and/or anti-CD137 agents. The ratio of anti-CD3 to anti-CD28 and anti-CD27 to anti-CD137 may be about 1:1000:1000, about 1:500:500, about 1:100:100, about 1:50:50, about 1:40:40, about 1:30:30, about 1:20:20, about 1:10:10, about 1:5:5, about 1:4:4, about 1:3:3, or about 1:2:2.

Following isolation, T cells may be activated by undisturbed incubation with compositions of the invention in culture for a period of 1-5 days, a period of 1-4 days, a period of 2-3 days, a period of about 2 days, or a period of about 3 days. Following undisturbed activation, the isolated T cells are expanded by adding fresh culture media and cytokines as required. Expansion may occur for a period of 7-20 days, a period of 8-15 days, a period of 10-14 days, a period of about 10 days, a period of about 11 days, a period of about 12 days, a period of about 13 days, or a period of about 14 days. Cytokines for T cell expansion are described above and discussed below in reference to the examples.

The expansion of T cells may be such that a set cell count of T cell subpopulation cells can be produced. For example, a T cell subpopulation comprising about 10⁶ cells, about 10⁷ cells, about 10⁸ cells, or about 10¹⁰ cells (e.g., from about 10⁶ to about 10¹⁰ cells, from about 10⁷ to about 10¹⁰ cells, from about 10⁸ to about 10¹⁰ cells, from about 10⁹ to about 10¹⁰ cells, from about 10⁶ to about 10⁹ cells, from about 10⁶ to about 10⁹ cells, etc.) may be produced by expanding T cells using methods and compositions of the present invention.

Using methods of the invention, activation/expansion in T cell subpopulations can be achieved in about 2 hours, about 4 hours, about 6 hours, about 8 hours, about 10 hours, about 12 hours, about 18 hours, about 24 hours, about 36 hours, about 48 hours, about 3 days, about 4 days, about 5 days, about 6 days, about 7 days, about 8 days, about 9 days, about 10 days, about 11 days, about 12 days, about 13 days, about 14 days about 20 days about 30 days, etc. (e.g., from about 2 hours to about 30 days, from about 8 hours to about 30 days, from about 20 hours to about 30 days, from about 1 day to about 30 days, from about 3 days to about 30 days, from about 5 days to about 30 days, from about 7 days to about 30 days, etc.).

In some instances, solid phases (e.g., beads) containing one or more stimulatory signal (e.g., anti-CD3 antibodies, anti-CD28 antibodies, interleukin 2, etc.) will not be removed from T cell populations after expansion. In other instances, once a T cell population has been expanded to a required level as described above, the expanded population can then be separated from the solid phase in an appropriate way. For example, if one of one or more stimulatory signal is attached to a solid phase and that solid phase is a tissue culture well, plate or bottle, the T cell population can be obtained by removal of the culture medium which contains the T cells. If the solid phase comprises for example magnetic beads, a magnetic field is used to attract the beads to the side of the vessel and the culture medium containing the T cells can then be, for example, poured off. Other particulate solid supports (e.g., non-magnetic beads) may be centrifuged or filtered away from the cells. Although the majority of T cells will typically be located in the culture medium, some T cells are likely to be attached to the solid phase after expansion. If desired, such T cells can be detached by, for example, resuspension using a pipette or other suitable means. Such a resuspension will normally be carried out before the T cells are separated from the solid phase to improve the yield. Once separated from the solid phase the T cell population can then be further treated and/or manipulated in any desired way or used directly for suitable applications such as for example in vitro experiments and research, non-therapeutic applications, therapeutic applications etc. as discussed below.

When soluble activators are employed, these may be removed, if desired, by competition with appropriate ligands, e.g., CD3 or CD28, but in many instances the T cells are collected from the culture medium and used for applications as described herein without further refinement. Conveniently, for large scale applications, appropriate isolation and preparation platforms may be used for selection of the T cell populations for stimulation and/or for the expansion protocol and/or isolation of the generated T cell population. In this regard, special mention may be made of DYNAL'S DYNAMAG™ CTS platforms in which closed sterile disposable bags may be used for any magnetic cell separation steps which are performed, e.g., for cell isolation prior to and DYNABEAD® removal after expansion.

If necessary the T cell population which is being expanded/activated according to the methods of the present invention may be re-stimulated by contacting the cells with further activators in a way similar to the initial stimulation. In general, re-stimulation is only necessary or desired if the T cells are to be cultured for a long period of time, e.g., more than 10-16 days.

Treg cells may be re-stimulated following expansion by incubation with the same bead as utilized for primary activation. Treg cells may be restimulated at about 16 days, 14 days, at about 11 days, at about 10 days, at about 9 days, at about 8 days, at about 7 days, at about 6 days, or at about 12 hours after primary activation.

The expansion in T cell subpopulations following methods of the invention may result in an increase in number that be relatively small, but is many instances the result is a significant increase, such as for example an increase in cell number of at least about 2 fold, preferably at least about 5 fold, about 20 fold, or about 50 fold and more preferably at least about 100 fold, about 500 fold, about 1,000 fold, about 2,000 fold, about 5,000 fold, about 10,000 fold, about 20,000 fold, about 30,000 fold, about 40,000 fold, about 50,000 fold, about 75,000 fold, about 100,000 fold, or greater. Such increases in number may be measured at any appropriate time point in the cell expansion protocol.

The present invention provides methods for generating one or more substantially pure population(s) of a T cell subpopulation. For purposes of the invention, a population of substantially pure T cell subpopulation contains about 10% or less of undesired cells (e.g., not the subpopulation cell type desired). For example, for purposes of illustration, if Treg cells are the T cell subpopulation desired, then a substantially pure Treg subpopulation would contain about 10% or less of cells that are not Treg cells. Of course, this also applies to other T cell subpopulations. In some embodiments, substantially pure can encompass about 25% or less, about 20% or less, about 15% or less, about 14% or less, about 13% or less, about 12% or less, about 11% or less, about 10% or less, about 9% or less, about 8% or less, about 7% or less, about 6% or less, about 5% or less, about 4% or less, about 3% or less, about 2% or less, and/or about 1% or less of T cell that are not members of the desired subpopulation(s).

In standard Th17 generation protocols (X-VIVO™15 media with polarizing cytokines) stimulatory solid supports (e.g., DYNABEADS®) are typically kept in culture during entire culture period. Process simplification would occur if the solid supports (e.g., beads) could be removed prior to the end of the culture period (e.g., 48 h or 72 h post-activation), especially where cells are contained in a small volume and cells are being prepared for viral transduction (gene modification). One way processes would be improved is that there would be no need for large-scale solid support (e.g., magnetic bead) removal at or near the end of culture.

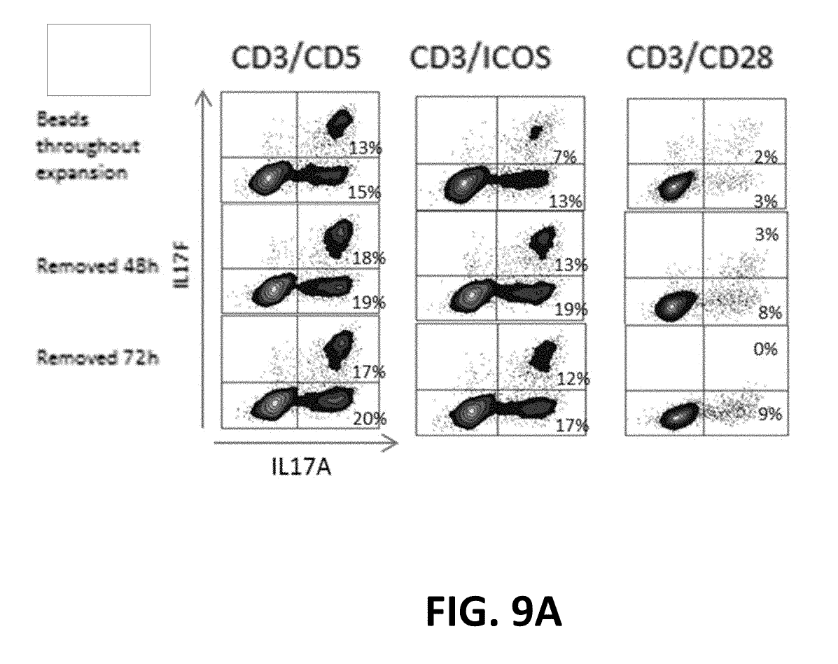

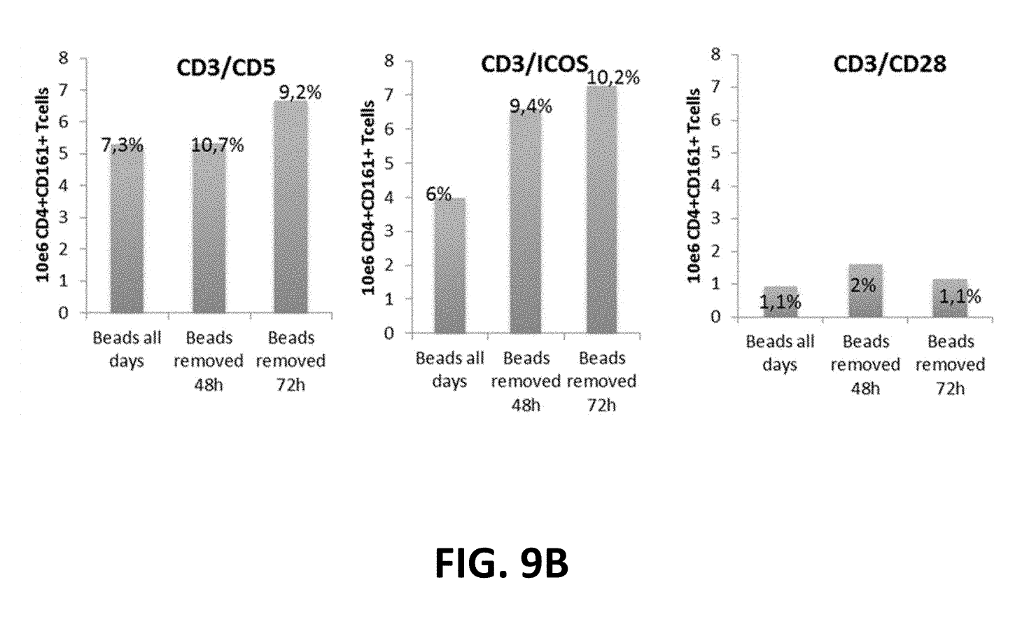

As shown in FIG. 9A and FIG. 9B, it has also been found that early activating support removal results in improved polarization of Th0 cells towards the Th17 phenotype. Experiments with early bead removal have also shown up to 200 fold expansion that is not affected by bead removal.

It has been observed that the effect of early bead removal on Th17 polarization is greater with ICOS co-stimulated cells than with CD5 co-stimulated cells, and almost negligible after CD28 co-stimulation (FIG. 9A and FIG. 9B).

In some instances, it may be desirable to remove solid supports (e.g., beads, such as magnetic beads) used to activate T cells from T cell mixtures. Further, in some instances, it may be desirable to remove such solid supports after a fairly short period of time (e.g., within 5 days). Thus, the invention includes compositions and methods for T cell activation where T cells are exposed to agents that stimulate one or more cell surface markers for less than five days (e.g., from about 1 day to about 5 days, from about 2 days to about 5 days, from about 3 days to about 5 days, from about 1 day to about 4 days, from about 2 days to about 3 days, etc.).

In a specific embodiment, multiple samples of a mixed population of T cells are exposed to (1) solid surfaces (e.g., magnetic beads) containing an anti-CD3 antibody and an anti-CD5 antibody, (2) IL-1β and (3) IL-6, (4) IL-23, and (5) TGF-β (and, optionally, an aryl hydrocarbon receptor agonist), under conditions that allow for T cell expansion. For one sample, the solid surface is not removed. For a second sample, the solid surfaces are removed after 1 day. For a third sample, the solid surfaces are removed after 2 days. For a fourth sample, the solid surfaces are removed after 3 day. Expansion of activated T cells may be performed essentially as set out in Example 10. At the end of the expansion process (about 14 days), the Th17 phenotype of the expanded cells, the number of Th17 cells, and the ratios of Th17 cells to other cell types are determined.

### III. Compositions of the Invention

Compositions of the invention comprise surfaces with immobilized agents in quantities, ratios, or combinations capable of stimulating expansion of specific T cell subpopulations. In alternative embodiments, the agents may be in solution.

Agents contemplated by the invention include protein ligands, and synthetic ligands. Agents that can bind to cell surface moieties, and under certain conditions, cause ligation and aggregation that leads to signaling include, but are not limited to, lectins (for example, phytohemagluttinin (PHA), lentil lectins, concanavalin A), antibodies, antibody fragments, peptides, polypeptides, glycopeptides, receptors, B cell receptor and T cell receptor ligands, MHC-peptide dimers or tetramers, extracellular matrix components, steroids, hormones (for example, growth hormone, corticosteroids, prostaglandins, tetra-iodo thyrohormone, corticosteroids, prostaglandins, tetra-iodo thyromine), bacterial moieties (such as lipopolysaccharides), mitogens, superantigens and their derivatives, growth factors, cytokines, adhesion molecules (such as, L-selectin, LFA-3, CD54, LFA-1), chemokines, and small molecules. The agents may be isolated from natural sources such as cells, blood products, and tissues, or isolated from cells propagated in vitro, prepared recombinantly, by chemical synthesis, or by other methods known in the art.

In one embodiment, agents of the invention include antibodies. Antibodies of the present invention include but are not limited to: Anti-CD3 (Thermo Fisher Scientific, Norway); Anti-CD137 (6B4: provided by the University of Navarra, Pamplona, Spain or 4B4-1 from Affymetrix/BioScience, CA, USA); Anti-CD28 (XR-CD28: Thermo Fisher Scientific, Norway); Anti-ICOS (ISA-3) (Affymetrix/BioScience, CA, USA), anti-CD2, anti-CD5, anti-CD6, anti-CD134, anti-CD40L, anti-CTLA-4, anti-GITR, anti-LFA-1, anti-SLAM, anti-CD27, anti-HVEM, anti-LIGHT, anti-DR3, anti-TIM1, anti-CD226.

Antibodies of the present invention can be obtained from public sources such as the American Type Culture Collection (ATCC), antibodies to T cell accessory molecules and cell surface proteins can be produced by standard techniques. Methodologies for generating antibodies for use in the methods of the invention are known in the art. Antibodies may also be produced as genetically engineered immunoglobulins (Ig) or Ig fragments designed to have specific desired properties. As a non-limiting example, antibodies may include a recombinant IgG that is a chimeric fusion protein having at least one variable (V) region domain from a first mammalian species and at least one constant region domain from a second distinct mammalian species. Most commonly, a chimeric antibody has murine variable region sequences and human constant region sequences. Such murine/human chimeric immunoglobulin may be “humanized” by grafting the complementarity determining regions (CDRs), which confer binding specificity for an antigen, derived from a murine antibody in a human-derived V region framework regions and human-derived constant regions. Antibodies containing CDRs of different specificities can also be combined to generate multi-specific (bi- or tri-specific, etc.) antibodies. Fragments of these molecules may be generated by proteolytic digestion, or optionally, by proteolytic digesting followed by mild reduction of disulfide bonds and alkylation, or by recombinant genetic engineering techniques.

Agents of the invention may further include any molecule that specifically binds to the cell surface receptors CD3, CD137, CD28, CTLA-4, GITR, ICOS (ISA-3), CD2, CD5, CD6, CD134, anti-CD40L, LFA-1, LFA-2, LFA-3. Agents of the invention may comprise peptides, small organic molecules, peptidomimetics, soluble T cell receptors, antibodies, or the like. Agents of the invention may be naturally occurring cell surface receptor ligands. Suitable agents of the invention may be identified by assays to determine affinity and specificity of binding that are known in the art, including competitive and non-competitive assays. Assays of interest include ELISA, RIA, flow cytometry, etc.

Agents contemplated by the present invention may further include protein ligands, natural ligands, and synthetic ligands. Agents that can bind to cell surface moieties, and under certain conditions, cause ligation and aggregation that leads to signalling include, but are not limited to, lectins (for example, PHA, lentil lectins, concanavalin A), antibodies, antibody fragments, peptides, polypeptides, glycopeptides, receptors, B cell receptor and T-cell receptor ligands, extracellular matrix components, steroids, hormones (for example, growth hormone, corticosteroids, prostaglandins, tetra-iodo thyronine), bacterial moieties (such as lipopolysaccharides), mitogens, antigens, superantigens and their derivatives, growth factors, cytokine, viral proteins (for example, HIV gp-120), adhesion molecules (such as, L-selectin, LFA-3, CD54, LFA-1), chemokines, and small molecules. The agents may be isolated from natural sources such as cells, blood products, and tissues, or isolated from cells propogated in vitro, or prepared recombinantly, or by other methods known to those with skill in the art.

In one aspect of the present invention, when it is desirous to stimulate T-cells, useful agents include ligands that are capable of binding the CD3/TCR complex, CD2, and/or CD28 and initiating activation or proliferation, respectively. Accordingly, the term ligand includes those proteins that are the “natural” ligand for the cell surface protein, such as a B7 molecule for CD28, as well as artificial ligands such as antibodies directed to the cell surface protein. Such antibodies and fragments thereof may be produced in accordance with conventional techniques, such as hybridoma methods and recombinant DNA and protein expression techniques. Useful antibodies and fragments may be derived from any species, including humans, or may be formed as chimeric proteins, which employ sequences from more than one species.

In alternative embodiments, a costimulatory signal may also include rapamycin. Rapamycin may be contacted with the cells prior to, simultaneously with, and/or subsequent to contact of the cells with the activators. Rapamycin may be present throughout the proliferation/expansion step in the method according to the invention. Rapamycin may be added in one or more steps. Thus for example, as described in the examples herein, isolated CD4+ cells may be stimulated with activators and rapamycin at the same time. In such methods, subsequent growth and passaging may be performed in the presence of rapamycin, but not the activators.

Rapamycin may be used at a concentration of from about 0.01 μM to about 10 μM, such as about 0.5 μM to about 2 μM, or about 1 μM. Rapamycin is a protein kinase inhibitor with a molecular weight of 914.2, also referred to as Sirolimus, Rapamune, AY-22989, RAPA and NSC-226080, available from Sigma, Calbio Chem, LC Labs etc. Rapamycin is available from a variety of commercial sources, such as A.G. Scientific, Inc. (San Diego, Calif., USA).

Other cytokines and/or growth factors may be added to the cultures as appropriate. Such cytokines and/or growth factors are added at appropriate concentrations and time points. For example, IL-2 and/or IL-4 may be added to enhance the proliferation of the T cells. Other cytokines may be added to induce particular differentiation patterns if required (e.g., TGF-β and IL-10). For example, IL-4 has been shown to trigger differentiation of T cell populations into the Th2 subpopulation and IFN-γ and IL-12 to trigger differentiation into the Th1 subpopulation (Sunder-Plassmann et al., Blood 87:5179-5184 (1996)). Thus in some embodiments of the invention, a cytokine, for example, IL-2 may be added at one or more steps to a final exogenously added concentration of 10-4,000 U/ml (e.g., from about 10 U/ml to about 3,000 U/ml, from about 10 U/ml to about 2,000 U/ml, from about 10 U/ml to about 1,500 U/ml, from about 15 U/ml to about 4,000 U/ml, from about 15 U/ml to about 3,000 U/ml, from about 15 U/ml to about 1,500 U/ml, from about 20 U/ml to about 2,000 U/ml, etc.). In some specific instances, IL-2 may be added at 20 U/ml during stimulation and at 1,000 U/ml periodically during the initial culture period and 20 U/ml in the period prior to harvest.

IL-4, for example, may be exogenously added, for example, at a final concentration of about 1,000-5,000 U/ml or about 100-10,000 U/ml (e.g., from about 1,000 U/ml to about 5,000 U/ml, from about 5,000 U/ml to about 5,000 U/ml, from about 250 U/ml to about 5,000 U/ml, from about 100 U/ml to about 5,000 U/ml, from about 800 U/ml to about 2,500 U/ml, etc.). In some instances, at about 1,000 U/ml may be added during stimulation and culture until harvest. Appropriate cytokines and growth factors and their effects on T cells are well known and described in the art. Once the T cell population has been brought into contact with the activators and, optionally, rapamycin under appropriate conditions for growth of the T cells, growth may be allowed to progress for a time period selected according to the final number of T cells required and the rate of expansion of the cells. Passaging of the cells may be undertaken during this period. Such a time period is normally between about 3 and about 10 days but can be as long as about 14 to about 20 days or even longer providing the viability and continued proliferation of the T cells is maintained.

Compositions of the invention may comprise a surface on which to immobilize the above described agents. A surface of the present invention may be any surface capable of having a ligand bound thereto or integrated into and that is biocompatible, that is, substantially non-toxic to the target cells to be stimulated. The biocompatible surface may be biodegradable or non-biodegradable. The surface may be natural or synthetic. A synthetic surface may be a polymer. The surface may comprise collagen, purified proteins, purified peptides, polysaccharides, glycosaminoglycans, extracellular matrix compositions, liposomes, or cell surfaces. A polysaccharide may include, by way of example, cellulose, agarose, dextran, chitosan, hyaluronic acid, or alginate. Other polymers may include polyesters, polyethers, polyanhydrides, polyalkylcyanoacryllates, polyacrylamides, polyorthoesters, polyphosphazenes, polyvinylacetates, block copolymers, polypropylene, polytetrafluorethylene (PTFE), or polyurethanes. The polymer may be lactic acid or a copolymer. A copolymer may comprise lactic acid and glycolic acid (PLGA). Non-biodegradable surfaces may include polymers such as poly(dimethylsiloxane) and poly(ethylene-vinyl acetate). Biocompatible surfaces include, by way of example, glass (e.g., bioglass), collagen, chitin, metal, hydroxyapatite, aluminate, bioceramic materials, hyaluronic acid polymers, alginate, acrylic ester polymers, lactic acid polymer, glycolic acid polymer, lactic acid/glycolic acid polymer, purified proteins, purified peptides, or extracellular matrix compositions. Other polymers comprise a surface may include glass, silica, silicon, hydroxyapatite, hydrogels, collagen, acrolein, polyacrylamide, polypropylene, polystyrene, nylon, or any number of plastic of synthetic organic polymers, or the like. The surface may comprise a biological structure, such as a liposome or cell surface, such as red blood cells (RBCs). The surface may be in the form of a lipid, a plate, bag, pellet, fiber, mesh, or particle. A particle may include, a colloidal particle, a microsphere, nonparticle, a bead, or the like. In the various embodiments, commercially available surfaces, such as beads, or other particles, are useful (e.g., Miltenyi Particles, Miltenyi Biotec, Germany; Sepharose beads, Pharmacia Fine Chemicals, Sweden; DYNABEADS®, Dynal Inc., New York; PURABEADS®, Prometric Biosciences).

Proteins, such as antibodies, may be conjugated to solid supports (e.g., beads) in any number of ways. One way to connect proteins to supports is through biotin and avidin or streptavidin. One system that has been developed is referred to as Strep-tag® (IBA GMBH, Gottingen, Germany). This system allows for the use of an eight amino acid sequence tag (Trp-Ser-His-Pro-Gln-Phe-Glu-Lys (SEQ ID NO: 1)) that binds to streptavidin. Further, such systems allow for the release of bound cells and proteins through competitive binding with biotin. Thus, the invention includes compositions and methods for the connection of proteins (e.g., antibodies) to solid supports by streptavidin interactions, as well as purification of cells bound to such solid supports. Cell purification methods include the following. A solid support and antibody interaction is formed by avidin or streptavidin, where the antibody binds to a cell surface marker (e.g., CD4). The solid supports are separated from unbound cells, then bound cells are released. Release may occur by such methods as proteolytic cleavage of the antibody or through competition with added biotin. Such supports may be used for cell purification, as well as T cell activation.

Similar products are available from Miltenyi Biotec (Bergisch Gladbach, Germany, cat. no. 130-090-485). The products comprise beads with anti-biotin bound thereto, which may be used to bind to biotinylated antibodies. Such beads may also be used for cell purification, as well as T cell activation.

When beads are used, the bead may be of any size that effectuates target cell stimulation. In some embodiments, beads from about 5 nm to about 500 μm in size are used. The choice of bead size will often depend on the particular use the bead will serve. For example, when using paramagnetic beads, the beads typically range in size below 1 μm to from about 2.8 μm to about 500 μm and generally from about 2.8 μm to about 50 μm. Lastly, one may choose to use super-paramagnetic nonparticles which can be as small as about 10⁻⁵ nm. An exemplary bead, the DYNABEADS® M-450, is 4.5 μm. Thus, beads used in the practice of the invention will generally have diameters from about 0.00001 nm to about 500 μm (e.g., from about 0.01 nm to about 500 μm, from about 1 nm to about 500 μm, from about 5 nm to about 500 μm from about 0.00001 nm to about 50 μm, from about 0.1 nm to about 50 μm, from about 10 nm to about 50 μm, from about 50 nm to about 50 μm, from about 50 nm to about 25 μm, from about 50 nm to about 1 μm, from about 10 μm to about 500 μm, from about 1 μm to about 10 μm, etc.

In some embodiments, DYNABEADS® may be used. DYNABEADS® have the advantage of allowing parameters such as ligand composition and stoichiometry to be varied for a systematic examination of their effects on the T cell activation and differentiation. DYNOSPHERES® (Thermo Fisher Scientific, Lillestrøm, Norway) may be conjugated with agents in varied stoichiometry for the expansion of specific T cell subpopulations. DYNOSPHERES® allow for the conjugation of antibodies or other agents of the invention to a bead without a linking group by conjugation to a naked bead coated with Epoxy groups.

An agent may be conjugated, attached, incorporated into, coupled to, or integrated into a surface by a variety of methods known and available in the art. The agent may be a natural ligand, a protein ligand, or a synthetic ligand. The attachment may be covalent or noncovalent, electrostatic, or hydrophobic and may be accomplished by a variety of attachment means, including by way of example, chemical, mechanical, enzymatic, electrostatic, or other means whereby a ligand is capable of stimulating the cells. The attachment of the agent may be direct or indirect (e.g., tethered). For example, the antibody may be attached directly to a surface (i.e., directly attached) or through indirect attachment (e.g., avidin or streptavidin/biotin).

With respect to cell surfaces, the attachment may be via genetic expression of the agent using any number of technologies known in the art, such as transfection or transduction, etc. of an expression vector comprising the coding region of the agent of interest. The antibody to the ligand may be attached to the surface via an anti-idiotype antibody. Another example includes using Protein A or Protein G, or other non-specific antibody binding molecules, attached to any appropriate surface, including DYNABEADS®, as a method for conjugating antibodies thereto. Alternatively, the ligand may be attached to the surface by chemical means, such as cross-linking to the surface, using commercially available cross-linking reagents (for example, those available from Pierce, Rockford, Ill.) or other means. In certain embodiments, ligands are covalently bound to the surface. Further, in one embodiment, commercially available tosyl-activated DYNABEADS® or DYNABEADS® with epoxy-surface reactive groups are incubated with the ligands of interested according to the manufacturer's instructions. Briefly, such conditions typically involve incubations in a phosphate or borate buffers from about pH 4 to pH 9.5 at temperatures ranging from about 4° to 37° C.

The activators, in appropriate ratios, are generally mixed together before they are put into contact with the beads. “Appropriate reaction conditions” can vary according to the particular activators used but exemplary conditions may comprise for example an incubation of the beads and activators, e.g., antibodies in a phosphate buffer (for example 0.5 M phosphate) of about pH 7.4 and a particle concentration of about 4×10⁸ beads/ml. Human serum albumin (e.g., recombinant HSA) or other serum albumin, such as bovine serum albumin, may optionally be present (e.g., at a concentration of about 0.05% w/v) to stabilize the activators and block any remaining hydrophobic patches on the surface of the beads.

Once the appropriate reaction mixture has been set up the reaction is allowed to progress for an appropriate time and under appropriate conditions to facilitate the activator absorption to the surface in the required ratio. Again the particular conditions can be varied depending on the components of the reaction mixture but exemplary conditions include incubation for about 6 to about 24 hours at about 37° C. with slow tilt and rotation.

Whatever the conditions chosen, once the immobilization reaction is complete the beads with activators immobilized to their surfaces in the appropriate ratio can be removed from the remaining aqueous medium by placing a magnet at the side of the reaction vessel and discarding the supernatant. The beads will generally be washed to remove any excess non-bound antibodies and are then ready for use. Such beads, once prepared, can be used immediately or can be stored for future use.

In some embodiments DYNABEADS® M-450 may be utilized for conjugation with surface acting agents. According to the manufacturer, DYNABEADS® M-450 are 4.5 micron epoxy beads. They are non-porous, monodisperse, superparamagnetic beads. High solution mobility allows the DYNABEADS® to interact with solution constantly for ease of antibody conjugation. The beads are further available with surface tosyl groups.

Agents may be immobilized on beads either on the same bead, i.e., “cis” or to separate beads, i.e., “trans.”

In alternative embodiments activating agents of the present invention may be in solution.

Activation and expansion of particular subpopulation of T cells can be targeted via the particular surface agents and ratios of the surface agents. In some embodiments, compositions of the invention for expansion of T cells (e.g., Treg cells) comprise anti-CD3 agents and anti-CD28 agents. T cell (e.g., Treg cell) expanding compositions of the invention may comprise low, mid or high relative levels of CD3 adhered to a surface. By way of example, low anti-CD3 agents may be present on a surface at concentrations of about 0.01 to about 0.04, about 0.05 to about 0.4, about 0.06 to about 0.4, about 0.07 to about 0.4, about 0.08 to about 0.4, about 0.09 to about 0.4, about 0.1 to about 0.4, about 0.2 to about 0.4, about 0.3, or about 0.34 units; mid anti-CD3 agents may be present on surfaces of the invention at concentrations of about 0.5 to about 1.5, about 0.6 to about 0.8, or about 0.75 units; high anti-CD3 agents be present on surfaces of the invention at concentrations of about 2 to about 4, about 3 to about 4, about 3.4, or about 3.41 units. In some embodiments, beads for the expansion of T cell (e.g., Treg cell) comprising a low relative level of anti-CD3 antibody in conjunction with the anti-CD28 costimulation are used.

In some embodiments, compositions of the invention for activation and expansion of T cells (e.g., memory T cells) comprise anti-CD3 agent, anti-CD137 agent, and anti-CD28 agent. T cell (e.g., memory T cell) expanding compositions of the invention may comprise low, mid-low, mid-high, or high relative levels of CD3 adhered to a surface. By way of example, low anti-CD3 agents may be present on surfaces of the invention at concentrations of about 0.005 to about 0.02, about 0.008 to about 0.015, or about 0.01 units; mid-low anti-CD3 antibody may be present on surfaces of the invention at concentrations of about 0.02 to about 0.08, about 0.03 to about 0.07, or about 0.05 units; mid-high anti-CD3 agents may be present on surfaces of the invention at concentrations of about 0.09 to about 0.16, about 0.1 to about 0.15, about 0.14 or about 0.142 units; and high anti-CD3 agents may be present on surfaces of the invention at concentrations of about 1 to about 2, about 1.3 to about 1.7, about 1.4 to about 1.6 or about 1.5 units. In some embodiments for the expansion of T cell (e.g., memory T cell), bead comprising a low relative level of anti-CD3 antibody in conjunction with anti-CD28 and anti-CD137 costimulation are used.

In some embodiments, compositions of the invention for activation and expansion of T cells (e.g., Th17 cells) comprise anti-CD3 antibody, and anti-ICOS antibody, anti-CD5 antibody, or anti-CD28 antibody. T cell (e.g., Th17 cell) expanding compositions of the invention may comprise low, mid, or high relative levels of CD3 adhered to a surface. By way of example, low anti-CD3 agents may be present on surfaces of the invention at concentrations of about 0.02 to about 0.1, about 0.03 to about 0.08, about 0.04 to about 0.07, about 0.05 to about 0.07, or about 0.06 units; mid anti-CD3 agents may be present on surfaces of the invention at concentrations of about 0.1 to about 0.5, about 0.2 to about 0.5, or about 0.3 units; high anti-CD3 agents may be present on surfaces of the invention at concentrations of about 1 to 2, about 1.3 to about 1.7, or about 1.5 units. In some embodiments for the expansion of T cell (e.g., Th17 cell), beads comprising a low relative level of anti-CD3 antibody in conjunction with anti-CD5, anti-CD28, and/or anti-ICOS costimulation are used.

Antibodies may be conjugated to solid supports (e.g., beads) by any number of means. Some methods for conjugating antibodies involve contacting antibodies with solid supports under conditions that allow for the covalent coupling of the antibodies to the solid supports. A commercial product that may be used to prepare antibody coupled beads is the DYNABEADS® Antibody Coupling Kit (Thermo Fisher Scientific, Cat. No. 14311D).

The DYNABEADS® Antibody Coupling Kit enables covalent coupling of antibodies, as well as other proteins such as lectins, functional enzymes, onto the surface of DYNABEADS® M-270 Epoxy. The surface epoxy groups allow for the coupling of proteins by primary amino and sulfhydryl groups. The beads may be coupled to “saturation” resulting in low background binding upon exposes to post-coupling processing.

The amount of protein used in a reaction mixture to saturate the beads will vary with the “carrying capacity” of the beads used. When DYNABEADS® M-270 Epoxy beads, as an example, are used, it is recommended that at least 10 μg of protein be used per mg of beads to ensure saturation. This is so because the carrying capacity of these beads typically varies between roughly 7 and 9 μg of protein per mg of beads. Further, antibody load may be adjusted on a per bead basis by altering the ratios of antibodies the beads are contacted with. Thus, antibody/bead coupling can be adjusted to alter the ratios of ligands present on the beads, with respect to each other, and/or the average total amount of ligand on each bead.

DYNABEADS® M-450 Epoxy (Thermo Fisher Scientific, Cat. No. 14011) may also be used in the practice of the invention. In the process here is for the coupling of 1 ml (4×10⁸) beads. About 200 μg antibody (Ab) should be used per 1 ml (4×10⁸) beads. One ml of washed, and resuspended beads are placed in a tube. The tube is then placed in a magnet for 1 min and the supernatant is discarded. The tube is then removed from the magnet and the beads are resuspended in Buffer A (0.1 M sodium phosphate buffer, pH 7.4-8.0 or 0.1 M sodium borate buffer, pH 7.6-9.5). The volume is then brought up to 1 ml by the addition of 200 μg of antibody(ies). The mixture is then incubated for 15 min and then BSA is added to 0.01-0.1% w/v. This mixture is then incubated for 16-24 hours at room temperature with gentle tilting and rotation. The tube is then placed in a magnet for 1 minute and the supernatant is discarded. 1 ml Buffer B (Ca²⁺ and Mg²⁺ free phosphate buffered saline (PBS), 0.1% bovine serum albumin (BSA) and 2 mM EDTA, pH 7.4) is then added. The mixture is then mixed and incubated for 5 minutes with gentle tilting and rotation. This washing procedure is repeated twice. The tube from the magnet and the beads are resuspended in 1 ml of Buffer B (4×10⁸ beads/ml).

Another product that may be sued in the practice of the invention is the PIERCE™ NHS-Activated Magnetic Beads (Thermo Fisher Scientific, cat. no. 88827). These beads have N-hydroxysuccinimide (NHS) functional groups on a blocked magnetic bead surface. They are superparamagnetic (no magnetic memory) and a nominal mean diameter of 1 μm (nominal). These beads have a density 2.0 g/cm3 and a binding capacity: ≥26 μg of rabbit IgG/mg of beads.

To begin with, the protein solution and magnetic beads are brought to room temperature, then 300 μl of magnetic beads are placed into a 1.5 ml microcentrifuge tube. The tube is placed into a magnetic stand, the beads are collected and the supernatant is discarded. 1 ml of ice-cold Wash Buffer A (ice-cold 1 mM hydrochloric acid) is introduced into the tube and gently vortexed for 15 seconds. The tube is placed into a magnetic stand, the beads are collected and the supernatant is discarded. 300 μl of protein solution is then added into the tube and the tube is then vortexed for 30 seconds. The mixture is incubated for 1-2 hours at room temperature on a rotator. During the first 30 minutes of the incubation, the tube is vortexed for 15 seconds every 5 minutes. For the remaining time, the tube vortexed for 15 seconds every 15 minutes until incubation is complete. The beads are collected with a magnetic stand and the flow-through is saved. 1 ml of Wash Buffer B (0.1 M glycine, pH 2.0) is added to the beads and the tube is vortexed for 15 seconds. The tube is placed into a magnetic stand, the beads are collected and the supernatant is discarded. The washing and collection is repeated once. 1 ml of ultrapure water is then added to the beads and the tube is vortexed for 15 seconds. The tube is placed into a magnetic stand, the beads are collected and the supernatant is discarded. 1 ml of Quenching Buffer (3 M ethanolamine, pH 9.0) is added to the beads and the tube is vortexed for 30 seconds. The tube is then placed on a rotator for 2 hours at room temperature. The tube is placed into a magnetic stand, the beads are collected and the supernatant is discarded. 1 ml of water is added to the tube, mixed well, the beads are collected and the supernatant is discarded. 1 ml of Storage Buffer (50 mM borate, 0.05% sodium azide, pH 8.5) is added to the tube, mixed well, the beads are collected and the supernatant is discarded. This wash step is repeated two additional times. 300 μl of Storage Buffer is added to the beads, mixed well, and stored at 4° C. until ready for use. The final concentration of the protein-coupled magnetic beads should be around 10 mg/ml.

Other beads that may be used in the practice of the invention are DYNABEADS® M-270 Carboxylic Acid (Thermo Fisher Scientific, cat. no. 14306D), DYNABEADS® M-450 Tosylactivated (Thermo Fisher Scientific, Cat. No. 14013), DYNABEADS® M-280 Tosylactivated (Thermo Fisher Scientific, cat. no. 14204), and DYNABEADS® MyOne™ Tosylactivated (Thermo Fisher Scientific, cat. no. 65502).

In many instances, solid supports (e.g., beads) used in the practice of the invention will be saturated with protein. The ratios of the proteins used may vary. For example, 100% of the total load may be ligand (e.g., antibody). In such instances, one ligand may be present or multiple ligands may be present. When multiple ligands are present, they may be present in the same ratio (1:1) or different ratios (e.g., 1:10). Exemplary ratios of ligands that may be used in the practice are set out elsewhere herein.

TABLE 2
Exemplary Antibody Mixtures

| | | | CD3/CD28 | Antibody |
|---|---|---|---|---|
| Antibody 1 | Antibody 2 | Protein | Ratio | Load |
| 1 μg CD3 | 9 μg CD28 | None | 1:9 | 100% |
| 2 μg CD3 | 8 μg CD28 | 10 μg HSA | 1:4 | 50% |
| 1 μg CD3 | 9 μg CD28 | 30 μg HSA | 1:9 | 25% |
| 5 μg CD3 | 5 μg CD28 | None | 1:1 | 100% |
| 5 μg CD3 | 5 μg CD28 | 30 μg HSA | 1:1 | 25% |

Total ligand load may also be adjusted. As illustrated in Table 2 using antibody ligands, solid supports may be loaded to less than 100% capacity. In many instances when this is done it will be desirable to block sites of ligand attachment from further reaction. One way of doing this is by contacting solid support with an agent (e.g., a protein) that does not have ligand binding activity relevant for the particular application (e.g., human serum albumin (HSA), bovine serum albumin (BSA), one or more antibodies that do not bind T cell receptors, etc.) (non-ligand agent) under conditions that allow for both the ligand(s) and non-ligand agent to attach with the solid support. This allows for the adjustment of different ligand ratios and different total signal ratios. A number of examples along these lines are set out in Table 2.

Total signal with respect to T cells may be adjusted in any number of ways. For example, if the signal per solid support (e.g., bead) is low, then each T cell may be contacted with more solid support surface. When beads are used, this means that the bead to T cell ratio may be adjusted. For example, if a bead is used that is 50% loaded with antibody, then twice as many beads may be used to obtain the same total signal as beads that are 100% loaded.

### IV. Methods of Using the Invention

T cell subpopulations of the invention can be used in any number of physiological conditions, diseases and/or disease states for therapeutic purposes and/or research/discovery purposes. Condition or disease typified by an aberrant immune response may be an autoimmune disease, for example diabetes, multiple sclerosis, myasthenia gravis, neuritis, lupus, rheumatoid arthritis, psoriasis or inflammatory bowel disease. Conditions in which immune suppression would be advantageous include conditions in which a normal or an activated immune response is disadvantageous to the mammal, e.g., allo-transplantation of, e.g., body fluids or parts, to avoid rejection, or in fertility treatments in which inappropriate immune responses have been implicated in failure to conceive and miscarriage. The use of such cells before, during, or after transplantation avoids extensive chronic graft versus host disease which may occur in patients being treated (e.g., cancer patients). The cells may be expanded immediately after harvest or stored (e.g., by freezing) prior to expansion or after expansion and prior to their therapeutic use. Such therapies may be conducted in conjunction with known immune suppressive therapies.

Once an appropriate T cell population or sub population has been isolated from a patient or animal, genetic or any other appropriate modification or manipulation may optionally be carried out before the resulting T cell population is expanded using the methods and supports of the invention. The manipulation may, for example, take the form of stimulate/re-stimulation of the T cells with anti-CD3 and anti-CD28 antibodies to activate/re-activate them.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 ml to 400 ml. In certain embodiments, T cells are activated from blood draws of about 20 ml, about 30 ml, about 40 ml, about 50 ml, about 60 ml, about 70 ml, about 80 ml, about 90 ml, or about 100 ml. The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention may be administered by i.v. injection. The compositions of T cells may be injected directly into a tumor, lymph node, or site of infection/inflammation (autoimmunity).

Compositions of T cell subpopulation generated according to the invention have many potential uses, including experimental and therapeutic uses. In particular it is envisaged that such T cell populations will be extremely useful in suppressing undesirable or inappropriate immune responses. In such methods a small number of T cells are removed from a patient and then manipulated and expanded ex vivo before reinfusing them into the patient. Examples of diseases which may be treated in this way are autoimmune diseases and conditions in which suppressed immune activity is desirable (e.g., for allo-transplantation tolerance). A therapeutic method could comprise providing a mammal, obtaining a biological sample from the mammal that contains T cells; expanding/activating the T cells ex vivo in accordance with the methods of the invention as described above; and administering the expanded/activated T cells to the mammal to be treated. The first mammal and the mammal to be treated can be the same or different. The mammal can generally be any mammal, such as cats, dogs, rabbits, horses, pigs, cows, goats, sheep, monkeys, or humans. The first mammal (“donor”) can be syngeneic, allogeneic, or xenogeneic. Therapy could be administered to mammals having aberrant immune response (such as autoimmune diseases including, for example diabetes, multiple sclerosis, myasthenia gravis, neuritis, lupus, rheumatoid arthritis, psoriasis, and inflammatory bowel disease), tissue transplantation, or fertility treatments.

The main technical hurdles involved in such therapies include the purification of the cells of interest from the patient and the expansion and/or the manipulation of the cells in vitro. Such therapies generally require a large number of cells and thus it can be seen that it is vital to optimize the methods of inducing in vitro T cell proliferation in order to maximize the number of T cells produced and minimize the time required to produce the T cells in sufficient numbers. The compositions and methods of the present invention provide large numbers of T cells of specific subpopulations.

T cell subpopulations of the present invention can be used in a variety of applications and treatment modalities. T cell subpopulations of the present invention can be used in the treatment of disease states including, but not limited to, cancer, autoimmune disease, allergic diseases, inflammatory diseases, infectious diseases, and graft versus host disease (GVHD). Broad example T cell therapies include infusion to a subject of T cell subpopulations externally selectively expanded by methods of the present invention following or not following immune depletion, or infusion to a subject of heterologous externally expanded T cells that have been isolated from a donor subject (e.g., adoptive cell transfer).

### Autoimmune Disorders

Autoimmune diseases or disorders are those diseases that result from an inappropriate and excessive response to a self-antigen. Studies have implicated defective Treg cells in autoimmune disorders. Autoimmune diseases include: diabetes mellitus, uveoretinitis and multiple sclerosis, Addison's disease, celiac disease, dermatomyositis, Grave's disease, Hashimoto' s thyroiditis, alopecia areata, ankylosing spondylitis, autoimmune hepatitis, autoimmune parotitis, hemolytic anemia, pemphigus vulgaris, psoriasis, rheumatic fever, sarcoidosis, scleroderma, spondyloarthropathies, vasculitis, vitiligo, myxedema, pernicious anemia, ulcerative colitis, Crohn's disease, dystrophic epidermolysis bullosa, epididymitis, glomerulonephritis, Graves' disease, Guillain-Barre syndrome, Myasthenia gravis, pernicious anemia, reactive arthritis, rheumatoid arthritis, Sjogren' s syndrome, and systemic lupus erthematosus as none limiting examples. In autoimmune disease states, the CD4⁺ CD25⁺ T regs may be present in decreased number or be functionally deficient. Tregs from peripheral blood having reduced capacity to suppress T-cell proliferation have been found in patients with multiple sclerosis (Viglietta et al., J. Exp. Med. 199:971-979 (2004).), autoimmune polyglandular syndrome type II (Kriegel et al., J. Exp. Med. 199:1285-1291 (2004).), type I diabetes (Lindley et al. Diabetes 54:92-929 (2005).), psoriasis (Sugiyama et al., J. Immunol. 174:164-173 (2005)), and myasthenia gravis (Balandina et al., Blood 105:735-741 (2005)).

Treatment of autoimmune disorders with T cell therapy may involve differing mechanisms. In one embodiment, blood or another source of immune cells can be removed from a subject inflicted with an autoimmune disorder. Methods of the invention discussed herein can be used to selectively expand T cell types other than memory T cells from the patient sample. Following removal and expansion of autologous cells, innapropriate memory T cells can be depleted within a subject in need thereof by known methods, including low dose total body radiation, thymic irradiation, antithymocyte globulin, and administration of chemotherapy. Illustrative chemotherapeutic agents of the present invention include but are not limited to campath, anti-CD3 antibodies, cytoxin, fludarabine, cyclosporine, FK506, mycophenolic acid, steroids, FR901228, and irradiation. Following depletion of the innappropriate memory T cells which are capable of recognizing self-antigens, the externally expanded autologous T cells can be readministered to the subject to reconstitute or restimulate their immune system.

Alternatively, or in addition to the above described treatment modalities, Treg cells can be isolated from sources including peripheral blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. These T cells can be can be selectively expanded using methods of the invention. These expanded Treg cells can be readministered to a patient to suppress inappropriate immune responses. This Treg therapy may be administered either to suppress the minimal remaining immune responses following immune depletion, or in subjects that have not undergone immune depletion.

### Graft Versus Host Disease

A major problem in hematopoietic stem cell transplantation is GVHD, which is caused by alloreactive T cells present in the infused hematopoietic stem cell preparation. In organ transplantation, graft rejection mediated by alloreactive host T cells is the major problem, usually overcome by long-term immunosuppression of the transplant recipient.

In methods similar to those described above, treating, reducing the risk of, or the severity of, an adverse GVHD event with T cell therapy may involve differing mechanisms. In one embodiment, blood or another source of immune cells can be removed from a subject inflicted with GVHD. Methods of the invention discussed herein can be used to selectively expand T cell types other than memory T cells, selectively expanding those cell types that do not comprise long-lasting recognition of antigens from the exogenous tissue. Following removal and external expansion of autologous cells, innapropriate memory T cells can be depleted within a subject in need thereof by known methods, including low dose total body radiation, thymic irradiation, antithymocyte globulin, and administration of chemotherapy. Illustrative chemotherapeutic agents of the present invention include but are not limited to campath. anti-CD3 antibodies, cytoxin. fludarabine, cyclosporine, FK506, mycophenolic acid, steroids, FR901228, and irradiation. Following depletion of the innappropriate memory T cells capable of recognizing antigens on the exogenous tissues, the externally expanded autologous T cells can be readministered to the subject to reconstitute or restimulate their immune system.

Alternatively, or in addition to the above described treatment modalities, Treg cells removed from patient blood can be selectively expanded. These expanded Treg cells can be readministered to a patient to suppress inappropriate immune responses, either to suppress the minimal remaining immune responses following immune depletion, or in subjects that have not undergone immune depletion.

### Allergic Diseases

Allergic diseases may also be affected by T cell dysfunction. Studies have indicated impaired CD4⁺ CD25⁺ Treg-mediated inhibition of allergen-specific T helper type 2 (Th2) are present in patients suffering seasonal allergies (Ling E M, et al., Lancet 2004; 363:608-15.; Grindebacke H, et al., Clin Exp Allergy 2004; 34:1364-72.). Furthermore, altered proportions of T cells populations have been implicated in individuals with allergies and asthmatic diseases compared to healthy subjects (Akdis M, et al., J Exp Med 2004; 199:1567-75.; Tiemessen M M, et al., J Allergy Clin Immunol 2004; 113:932-9.).

In methods similar to those described above, treatment, prevention, or alleviation of allergic diseases with T cell therapy may involve differing mechanisms. Similar to autoimmune disorders and GVHD, allergic diseases are caused by an innappropriate immune response. Suppression of that response, or depletion of cells capable of recognizing the inappropriate antigen may alleviate the allergic symptoms. In methods of T cell therapy for the treatment of allergies, blood can be removed from a subject suffering from an allergic disorder. Methods of the invention discussed herein can be used to selectively expand non T memory cell T cell types, selectively expanding those cell types that do not comprise long-lasting recognition of antigens from the inapprporiate antigen (e.g., a legume protein). Following removal and expansion of autologous cells, innapropriate memory T cells can be depleted within a subject in need thereof by known methods, including low dose total body radiation, thymic irradiation, antithymocyte globulin, and administration of chemotherapy. Illustrative chemotherapeutic agents of the present invention include but are not limited to campath, anti-CD3 antibodies, cytoxin, fludarabine, cyclosporine. FK506, mycophenolic acid, steroids, FR901228, and irradiation. Following depletion of the innappropriate memory T cells capable of recognizing antigens on the exogenous tissues, the externally expanded autologous T cells can be readministered to the subject to reconstitute or restimulate their immune system.

Alternatively, or in addition to the above described treatment modalities, Treg cells removed from patient blood can be can be selectively expanded. These expanded Treg cells can be readministered to a patient to suppress inappropriate immune responses, either to suppress the minimal remaining immune responses following immune depletion, or in subjects that have not undergone immune depletion.

### Inflammatory Diseases

T cell therapy has been implicated in the treatment of inflammatory diseases and inflammation associated disorders. Many of these diseases can also be categorized as autoimmune disorders. Non-limiting examples of inflammatory diseases and inflammation associated disorders include: diabetes; rheumatoid arthritis; inflammatory bowel disease; familial mediterranean fever; neonatal onset multisystem inflammatory disease; tumor necrosis factor (TNF) receptor-associated periodic syndrom (TRAPS); deficiency of interleukin-1 receptor antagonist (DIRA); and Behcet's disease.

Because of the role of Treg cells in suppressing inappropriate immune responses to non pathogenic antigens, decreased numbers or imparied functioning of these T cell subpopulations can contribute to inflammatory diseases. This is true of, for example, inflammatory bowel disease (M Himmell, et al., Immunology 2012 June; 136(2): 115-122) and rheumatoid arthritis (M Noack, et al., Autoimmunity Reviews 2014 June; 13(6): 668-677).

Inflammatory diseases are mechanistically similar to autoimmune disorders. As such, infllamatory diseases can be caused in part by an innappropriate immune response. Suppression of that response, or depletion of cells capable of recognizing the inappropriate antigen may alleviate the inflammatory symptoms. In methods of T cell therapy for the treatment of inflammatory diseases, blood can be removed from a subject suffering from an inflammatory disorder. Methods of the invention discussed herein can be used to selectively expand non T memory cell T cell types, selectively expanding those cell types that do not comprise long-lasting recognition of inapprporiate antigens (e.g., carbamylated proteins in anticarbamylated protein (anti-CarP) antibody mediated rheumatoid arthritis). Following removal and expansion of autologous cells, innapropriate memory T cells can be depleted within a subject in need thereof by known methods, including low dose total body radiation, thymic irradiation, antithymocyte globulin, and administration of chemotherapy. Illustrative chemotherapeutic agents of the present invention include but are not limited to campath, anti-CD3 antibodies, cytoxin, fludarabine, cyclosporine, FK506, mycophenolic acid, steroids, FR901228, and irradiation. Following depletion of the innappropriate memory T cells capable of recognizing self-antigens and mounting the resultant inflammatory response, the externally expanded autologous T cells can be readministered to the subject to reconstitute their immune system.

Alternatively, or in addition to the above described treatment modalities, Treg cells removed from patient blood can be can be selectively expanded. These expanded Treg cells can be readministered to a patient to suppress inappropriate immune responses, either to suppress the minimal remaining immune responses following immune depletion, or in subjects that have not undergone immune depletion.

### Hyperproliferative Disorders

Evidence from cancer patients has further implicated T cell dysfunction with hyperproliferative disorders, including cancer. For example, increased Treg activity may result in poor immune response to tumor antigens and contribute to immune dysfunction. Elevated populations of CD4+ CD25+ have been found in lung, pancreatic, breast, liver and skin cancer patients, in either the blood or tumor itself (Woo E Y, et al.; J Immunol 2002; 168:4272-6.; Wolf A M, et al. Clin Cancer Res 2003; 9:606-12.; Liyanage U K, et al. J Immunol 2002; 169:2756-61; Viguier M, et al. J Immunol 2004; 173:1444-53.; Ormandy L A, et al. Cancer Res 2005; 65:2457-64.).

Using the methods of the invention T cells specific for tumor antigens or hyperproliferative disorder antigens or antigens associate with a hyperproliferative disorder can be expanded. Tumor antigens are proteins that are produced by tumor cells that elicit an immune response, particularly T-cell mediate immune responses.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated by products of the invention include but are not limited to carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. Among these are cancers including skin cancer, brain cancer and other central nervous system cancers, head cancer, neck cancer, muscle/sarcoma cancer, bone cancer, lung cancer, esophagus cancer, stomach cancer, pancreas cancer, colon cancer, rectum cancer, uterus cancer, cervix cancer, vagina cancer, vulva cancer, penis cancer, breast cancer, kidney cancer, prostate cancer, bladder cancer, or thyroid cancer or glioblastoma.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia, and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia, and myelodysplasia.

Solid tumors are abnormal masses that usually do not contain cysts or liquid areas. Solid tumors can be benign or malignant. Different types of solid tumors are named for the types of cells that form them (such as sarcomas, carcinomas, and lymphomas). Examples of solid tumors such as sarcomas and carcinoma, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

One approach to treating subjects in need thereof or patients is to use the expanded T cells of the invention and genetically modify the T cells to target antigens expressed on tumor cells through the expression of chimeric antigen receptors (CARs). CARs are antigen receptors that are designed to recognize cell surface antigens in a human leukocyte antigen independent manner. In treatment utilizing CARs immune cells may be collected from patient blood or other tissue. The T cells are engineered as described below to express CARs on their surface, allowing them to recognize specific antigens (e.g., tumor antigens). These CAR T cells can then be expanded by methods of the present invention and infused into the patient. In certain embodiments, T cells are administered at 1×10⁵, 1×10⁶, 1×10⁷, 1×10⁸, 5×10⁸, 1×10⁹, 5×10⁹, 1×10¹⁰, 5×10¹⁰, 1×10¹¹, 5×10¹¹, or 1×10¹² cells to the subject. Following patient infusion, the T cells will continue to expand and express the CAR, allowing for the mounting of an immune response against cells harboring the specific antigen the CAR is engineered to recognize.

In one embodiment, the invention provides a cell (e.g., a T cell) engineered to express a CAR wherein the CAR T cell exhibits an antitumor property. The CAR of the invention can be engineered to comprise an extracellular domain having an antigen binding domain fused to an intracellular signaling domain of the T cell antigen receptor complex zeta chain (e.g., CD3 zeta). The CAR, when expressed in a T cell is able to redirect antigen recognition based on the antigen binding specificity.

The antigen binding moiety of the CAR comprises a target-specific binding element otherwise referred to as an antigen binding moiety. The choice of moiety depends on the type and number of ligands that define the surface of a target cell. For example, the antigen binding domain may be chosen to recognize a ligand that acts as a cell surface marker on target cells associated with a particular disease state. Thus the antigen moiety domain in the CAR of the invention include those associated with viral, bacterial and parasitic infections, autoimmune disease and cancer cells.

The expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The T cells of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements (e.g., enhancers) regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Methods of making CAR T cells are known in the art (see, e.g., U.S. Pat. No. 8,906,682).

In an embodiment where a T cell is a CAR T cell, the selection of the antigen binding moiety of the invention will depend on the particular type of cancer to be treated. Tumor antigens are known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RUL RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, surviving and telomerase, prostate-carcinoma tumor antigen-1 (PCTA-1), MAGE, ELF2M, neutrophil elastase, ephrin B2, CD22, insulin growth factor (IGF-1), IGF-II, IGF-I receptor and mesothelin.

In one embodiment, the tumor antigen comprises one or more antigenic cancer epitopes associate with a malignant tumor. Malignant tumors express a number of proteins that can serve as a target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecular such as the oncogene HER-2/Neu/ErbB-2. Yet another group of target antigens are onco-fetal antigens such as carcinoembryonic antigen (CEA). In B-cell lymphoma, the tumor-specific immunoglobulin constitutes a truly tumor-specific immunoglobulin antigen that is unique to the individual tumor. B-cell differentiation antigens such as CD19, CDd20; ROR1, CD22, CD23, λ/κ light chains are other candidates for target antigen in B-cell lymphoma.

The type of tumor antigen referred to may also be a tumor specific antigen (TSA) or tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond, or they may be antigens that are normally present at extremely low levels on normal cells but which are expressed at much higher levels on tumor cells.

Non-limiting examples of TSA or TAA antigens include the following: Differentiation antigens such as MART-1/MelanA (MART-1), gp100 (Pme117), tyrosinanse, TRP-1, TRP-2 and tumor-specific mutilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL. E2A-PRL, H4-RET, IGH-IGK, MYL-RAR; and viral antigens such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-catenin, CDK4, Mum-1, p 15, p 16, 43-9F, 5T4, 791Tgp72, alpha-fetoprotein, beta-HCG, BCA224, BTAA, CA 125, CA 15-3\CA 27/29\BCAA, CA 195, CA 242, CA-50, CAM43, CD68\P1, CO-029, FGF-5, C250, GA733\EpCAM, HTgp-175, M344, MA-50, MG7-Ag, MOV18, NB/70K, NY-CO-1, RCAS1, SDCCAG16, TA-90, MAC-2 binding protein\cyclophillin C-associated protein, TAA16, TAG72, TLP, and TPS, CD19, CD20, CD22, ROR1, Mesothelin, CD33/IL3Ra, c-met, PSMA, Glycolipid F77, EGRvIII, GD-2, MY-ESO-1 TCR, MAGE A3 TCR, and others.

### Infectious Diseases

The immune response to infectious diseases involves a balance of anti-pathogen and anti-inflammatory responses. T cells are heavily involved in this intricate balance. Infectious pathogens capable of eliciting a T cell response may be bacterial, viral, parasitic or fungal. Treg cells have been implicated in contributing to the chronicity of infection by Helicobacter pylori (Lundgren A, et al. Infect Immun 2003; 71:1755-62.), hepatitis B virus (HBV), and hepatitis C virus (HCV) (Cabrera R, et al., Hepatology 2004; 40:1062-71.; Stoop J N, et al. Hepatology 2005; 41:771-8.; Sugimoto K, et al., Hepatology 2003; 38:1437-48.). This elevation in a particular T cell subpopulation may contribute to the prolonged nature of these infections by inapproprirately suppressing memory T cell responses. The compositions of the present invention may be utilized to specifically expand a particular T cell subpopulation and could be utilized in the treatment of such infectious diseases.

Infectious disease can be caused by direct contact with a pathogen and spread from person to person, animal to person, or from mother to unborn child. Infectious diseases can alternatively spread through indirect contact, e.g., from contact with an infected surface such as door handle, table, counter or faucet handle. Infectious diseases can further be spread via insect bites or food contamination. Certain autoimmune disorders, such as HIV or AIDS, and some cancers can increase susceptibility to infectious diseases. Certain treatment regimens that supress the immune system can also enhance susceptibility to infectious diseases. Example infectious diseases include: smallpox, malaria, tuberculosis, typhus, plague, diphtheria, typhoid, cholera, dysentery, pneumonia.

As described above, several mechanisms exist by which selective expansion of T cells may be used in the treatment of disease states. In infectious disease states, a patient suffering from the infection does not have sufficient immunity to the infectious agent. Methods of the present invention may be used to selectively expand heterologous T memory cells from a donor with immunity to a particular infectious agent and utilized in adoptive T-cell transfer. The externally expanded T cells from an infectious agent experienced donor can then be infused into a patient inflicted with the infection. In certain embodiments, T cells are administered at 1×10⁵, 1×10⁶, 1×10, 1×10⁸, 5×10⁸, 1×10⁹, 5×10⁹, 1×10¹⁰, 5×10¹⁰, 1×10¹¹, 5×10¹¹, or 1×10¹² cells to the subject. The infectious antigen competent donor memory T cells can then aid in mounting an autologous immune response within the patient.

Alternative methods of utilizing the present invention in the treatment of infectious diseases include the selective expansion of autologous or heterologous Th17 cells for reinfusion or adoptive cell transfer respectively. Using methods described herein, T cells can be externally expanded from patient isolated blood or tissue. These expanded T cells can then be infused to the patient to aid in induction of B cells to secrete antibodies against the particular infectious antigen (e.g., Streptococci M-protein, Neisseria pilli, Borrelia burgdorferi lipoprotein VisE, B. pseudomallei polysaccharide antigens, Aspergillus fumigatus galactomannan, or F. tularensis lipopolysaccharide). In certain embodiments, T cells are administered at 1×10⁵, 1×10⁶, 1×10⁷, 1×10⁸, 5×10⁸, 1×10⁹, 5×10⁹, 1×10¹⁰, 5×10¹⁰, 1×10¹¹, 5×10¹¹, or 1×10¹² cells to the subject.

### Co-Therapies

Existing treatments may be recommended for many of the above listed disease states. T cell subpopulations expanded by the present invention may be used as sole replacement therapy in some cases or in conjunction with other known therapies. T cell therapies may be administered prior to, concurrently with, or following administration of other therapies.

The methods of the present invention may also be utilized with vaccines to enhance reactivity of the antigen and enhance in vivo effect. In one embodiment, the compositions of the present invention are administered to a patient in conjunction with a composition that enhances T cells in vivo, for example, IL-2, IL-4, IL-7, IL-10, IL-12, and/or IL-15. Further, given that T cells expanded by the present invention have a relatively long half-life in the body, these cells could act as perfect vehicles for gene therapy as described above, by carrying a desired nucleic acid sequence of interest and potentially homing to sites of cancer, disease, or infection. Accordingly, the cells expanded by the present invention may be delivered to a patient in combination with a vaccine, one or more cytokines, one or more therapeutic antibodies, etc. Virtually any therapy that would benefit by a more robust T cell population could be used in conjunction with the compositions of the present invention.

### V. Kits of the Invention

Also provided herein are kits comprising (i) compositions for the isolation of T cells from a subject; (ii) compositions for the ex vivo culture of T cells (iii) compositions for the selective expansion of one or more T cell subpopulation (e.g., Th17, Treg, memory T cells, etc.). Kits of the invention may optionally include compositions for the re-activation of Treg cells.

Kits can also include written instructions for use of the kit, such as instructions for wash steps, culturing conditions and duration of incubation of isolated T cells with compositions of the invention for selective expansion of specific T cell subpopulations.

It is intended that every maximum numerical limitation given throughout this specification includes every lower numerical limitation, as if such lower numerical limitations are expressly written herein. Every minimum numerical limitation given throughout this specification will include every higher numerical limitation, as if such higher numerical limitations are expressly written herein. Every numerical range given throughout this specification will include every narrower numerical range that falls within such broader numerical range, as if such narrower numerical ranges are all expressly written herein.

### EXAMPLES

The invention can be further understood by reference to the following examples, which are provided by way of illustration and are not meant to be limiting.

### Example 1

### Primary T Cells and Culturing Conditions

Peripheral blood mononuclear cells (PBMCs) are isolated from healthy donor buffy coats or aphaeresis (HemaCare Corp., CA USA, and Astarte Biologics, LLC, WA, USA) under informed consent. Antigen specific memory T cells are enriched in PBMCs from cytomegalovirus (CMV) positive donors after stimulation with CMV pp65 peptide NLVPMVATV (Astarte Biologics LLC). Regulatory T cells (Tregs) are obtained from elutriated and CD4-enriched (DYNABEADS® UNTOUCHED™ Human CD4 T cells, Cat. No. 11346D, Thermo Fisher Scientific, CA USA) lymphocyte fraction by fluorescence-activated cell sorting based on antibody labeling of CD4, CD25 and CD127 resulting in CD4⁺CD25⁺CD127^{low/-} expressing Treg cells (Oslo University Hospital, Ulleval Blood Bank Oslo, Norway).

T cells are activated either directly from PBMCs or from isolated or enriched T-cell subsets by using DYNABEADS® with various ligand compositions and stoichiometry as described. Activated T cells are cultured at 37° C. and 5% CO² in X-VIVO™ 15 (Lonza Biologics, MD, USA, Cat. No. BE04-418F) plus 2-5% CTS™ Immune Cell SR and 0.25 μg/ml Gentamicin (both Thermo Fisher Scientific NY, USA, Cat. No. 15710-049) and expansion is achieved by adding cytokines and fresh medium every 1-3 days to maintain a cell concentration of 0.5-2×10⁶ cells/ml.

Flow sorted T regulatory cells (Tregs) are expanded in medium containing 100 ng/ml rapamycin (PHZ1235 Thermo Fisher Scientific, NY USA) and 300 IU IL-2/ml (Thermo Fisher Scientific, NY USA), while CMV stimulated T cells are expanded in 100 IU IL-2/ml. Th17/Tc17 cells are expanded in medium containing polarizing cytokines (IL-6, IL-1β, IL-23, and TGF-β, all Thermo Fisher Scientific CA USA) in presence of anti-IL-4 and anti-IFN-γ neutralizing antibodies (Thermo Fisher Scientific, CA US) as described in Paulos et al. (Paulos et al., Science Transl. Med 55:55ra78 (2010)). 100 IU IL-2/ml is added day 3 post-activation.

### Example 2

### Generation of DYNABEADS®-Based Expansion Platforms with Various Ligand Compositions and Ratios

DYNOSPHERES® MS-4.5-REK particles (Thermo Fisher Scientific, Lillestrøm, Norway) are conjugated with different amounts and ratios of stimulatory and costimulatory ligands optimized for the activation and polarization of various T cell subsets i) Tregs, ii) antigen experienced memory T cells, iii) and Th17/Tc17 cells respectively. The relative amounts of stimulatory anti-CD3 antibody and its co-conjugated costimulatory ligand are summarized in Table 3.

TABLE 3
a) Dynabeads ® CD3/CD28 Treg expander

| DYNABEADS ® | CD3 (XR-CD3) | |
|---|---|---|
| conjugation | (units) | Costimulation (units) |
| DYNABEADS ®CD3/ | 0.34 | 3.4 CD28 (XR-CD28) |
| CD28 (low) | | |
| DYNABEADS ®CD3/ | 0.75 | 1.5 CD28 (XR-CD28) |
| CD28 (mid) | | |
| DYNABEADS ®CD3/ | 3.41 | 0.34 CD28 (XR-CD28) |
| CD28 (high) | | |

b) DYNABEADS ® T antigen-specific expander

| Dynabeads conjugation | CD3 (XR-CD3) | Costimulation |
|---|---|---|
| DYNABEADS ®CD3/ | 0.01 | 1.5 CD28 (XR-CD28), |
| CD137/CD28 (low) | | 1.5 CD137 (6B4) |
| DYNABEADS ®CD3/ | 0.01 | 1.5 CD137 (6B4) |
| CD137 (low) | | |
| DYNABEADS ®CD3/ | 0.01 | 1.5 CD28 (XR-CD28) |
| CD28 (low) | | |
| DYNABEADS ®CD3/ | 0.05 | 2.95 CD137 (6B4) |
| CD137 (mid-low) | | |
| DYNABEADS ®CD3/ | 0.142 | 1.42 CD28 (XR-CD28), |
| CD137/CD28 (mid-high) | | 1.42 CD137 (6B4) |
| DYNABEADS ®CD3/ | 1.5 | 1.5 CD28 (XR-CD28) |
| CD28 (high, CTS bead) | | |

c) DYNABEADS ™ CD3/ICOS Th17 expander

| | CD3 (XR-CD3) | |
|---|---|---|
| Dynabeads conjugation | units | Costimulation |
| DYNABEADS ®CD3/ | 0.06 | 2.94 ICOS/CD278 (ISA-3) |
| ICOS (low) | | |
| DYNABEADS ®CD3/ | 0.3 | 2.7 ICOS/CD278 (ISA-3) |
| ICOS (mid) | | |
| DYNABEADS ®CD3/ | 1.5 | 1.5 ICOS/CD278 (ISA-3) |
| ICOS (high) | | |
| DYNABEADS ®CD3/ | 1.5 | 1.5 CD28 (XR-CD28) |
| CD28 (high, CTS bead) | | |

Anti-CD3 and anti-CD28 antibodies XR-CD3 and XR-CD28 (Thermo Fisher Scientific, Norway), Anti-ICOS antibody ISA-3 (Affymetrix/eBioScience CA USA), Anti-CD137 antibody 6B4 (University of Navarra, Pamplona, Spain)

### Example 3

### Stimulation and Expansion of T Cells

T cells are activated either directly from PBMCs or from isolated or enriched T cell subsets by adding one DYNABEADS® per T cell, unless other ratio is stated. In most experiments DYNABEADS® CD3/CD28 CTS™ (CD3 high) is included as a reference stimulator. Additionally, an alternative Treg activation reagent (MACS GMP ExpAct Treg Kit; Miltenyi Cat. No 170-076-119) included in experiments to assess the level of Treg expansion. Activated T cells are left undisturbed for 2-3 days and thereafter expanded for 10-14 days by adding fresh culture media and cytokines as required. Tregs are re-stimulated day 9 utilizing the same stimulatory bead as the primary activation (day 0). Cells are analyzed on a Coulter Counter (Beckman Coulter, CA USA) in order to measure absolute cellular expansions.

### Example 4

### Antibodies and Flow Cytometry Analysis

The following antibodies are used for the Treg analysis: CD4 PerCP (Invitrogen, MHCD0431), CD25 APC (Invitrogen, MHCD2505) and CD127 PE (Invitrogen, A18684). Intracellular FOXP3 expression is analyzed by Alexa Fluor 488 (BD560047) following fixation/permeabilization (eBioscience). Flow cytometric data are collected on a BD LSRII (BD Biosciences) and analyzed with FACS Diva software (BD Biosciences). Lymphocytes are gated according to their forward and size scatter characteristics. Appropriate irrelevant isotype controls are used to set negative gates (IgG2a PerCP (Thermo Fisher Scientific, Cat. No. MA1-10426), IgG1 APC (Thermo Fisher Scientific, Cat. No. MG105), IgG1 PE (Thermo Fisher Scientific, Cat. No. MG104), and IgG1 Alexa Fluor 488 (BD Biosciences, Cat. No. 557702)).

CMV-specific CD8+ T cells are identified using Pro5 Pentamer APC (NLVPMVATV/A*02:01) (ProImmune, Oxford UK) and CD8-PE (Thermo Fisher Scientific, Cat. No. MHCD0804), with appropriate isotype controls (IgG1 PE, Thermo Fisher Scientific, Cat. No. MG104).

Intracellular IL-17 expression in PMA/Ionomycin activated Th17 cells are detected using IL-17A-PE (cat no. A18695, clone: 4H1524, Molecular Probes) and appropriate isotype controls (IgG2b PE, Cat No. 400313 BioLegend) following fixation/permeabilization (eBioscience). Flow cytometric data are collected on a BD LSRII (BD Biosciences) and analyzed with FACS Diva software (BD Biosciences).

### Example 5

### Functionality by Flow Cytometry

Th17 polarized and expanded T cells are characterized by their intracellular IL-17 expression 4-5 hours after PMA/Ionomycin (day 13 post-DYNABEADS® activation) stimulation using the Cell Stimulation Cocktail Kit containing protein transport inhibitors Brefeldin and Monensin (eBioscience, Cat. No. 00-4971). The stimulation is performed according to the protocol provided by eBioscience. IL-17 expression is assessed by intracellular IL-17 staining by flow cytometry as described.

### Example 6

### Activation and Expansion of Tregs

As shown in FIG. 1, CD4⁺CD²⁵⁺CD127^{low/-} flow cytometry sorted Tregs (˜90% FOXP3+ cells) are activated with DYNABEADS® Treg prototypes efficiently and expanded several hundred fold (upper) and retained high FOXP3 expression (lower) after 14 days in expansion culture. Cells are re-stimulated using the same prototype bead at day 9. Increased expansion is achieved with DYNABEADS® conjugated with a lower CD3 amount. As seen in FIG. 1, the stimulation protocol utilizing DYNABEADS®CD3/CD28 (low CD3—about 0.34) results in the highest Treg expansion. DYNABEADS® CD3/CD28 (high CD3—about 3.4) does not produce any effective Treg expansion. DYNABEADS® CD3/CD28 low (CD3—about 0.34) and DYNABEADS® CD3/CD28 mid (CD3—about 0.75) perform similar or better to the alternative Treg Stimulation Reagent. DYNABEADS® CD3/CD28 CTS™ (CD3—about 1.5) result in suboptimal expansion.

### Example 7

### Activation and Expansion of Antigen Experienced (CMV Specific Memory) T Cells

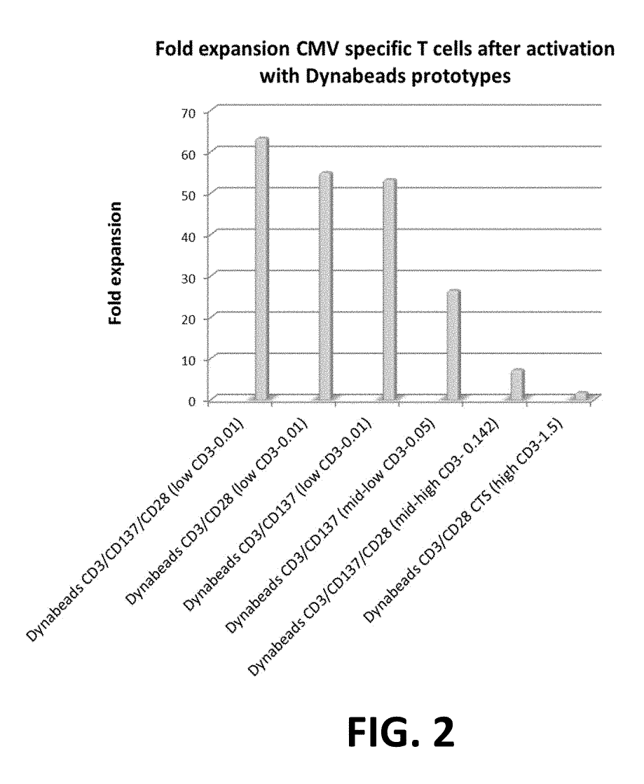

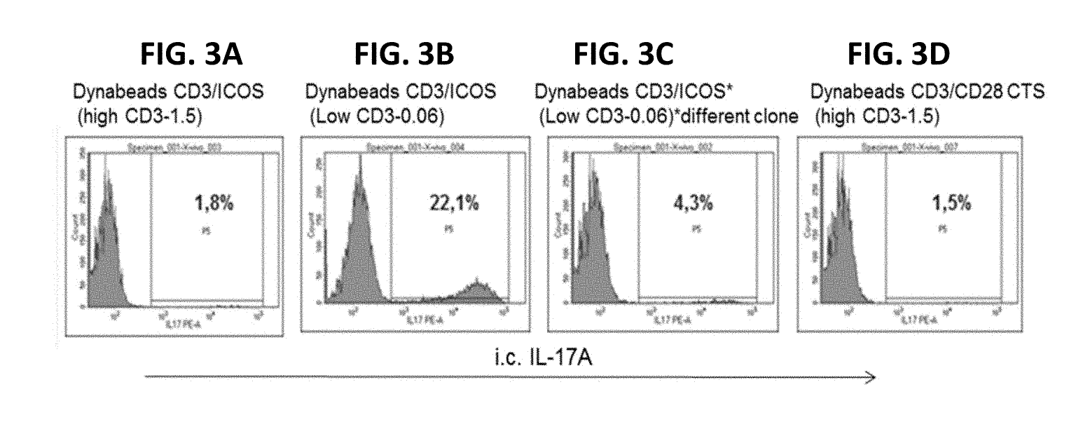

As seen in FIG. 2, fold expansion of CMV specific memory T cells at day 10 post-activation negatively correlates with increased signal strength provided by DYNABEADS® prototypes conjugated with increasing amounts of the agonistic CD3 antibody. By using cytomegalovirus (CMV) specific T cells as a model, growth kinetics and phenotypes are compared for T cells expanded from peptide stimulated PBMCs from CMV+ donors using different DYNABEADS® prototypes conjugated with CD3, CD28 and/or CD137 antibodies, and DYNABEADS® CD3/CD28 CTS™. The superiority of beads conjugated with low CD3 amounts (provide weaker CD3 activation signal) is shown herein. DYNABEADS® CD3/CD28 CTS™ is suboptimal and does not expand the CMV specific T cells. DYNABEADS® CD3/CD137/CD28 give the highest memory T cell expansion.

### Example 8

### Activation, Polarization and Expansion of Th17 Cells

T cells are cultured with Th17-polarizing conditions and expanded with DYNABEADS® conjugated with antibodies against CD3/ICOS (various amounts of anti-CD3 antibody conjugated; 1.5 and 0.06, and two different ICOS clones) or CD3/CD28 (CTS bead). Starting on day 3, IL-2 is added to the cultures. At day 13 cultures are stimulated with PMA-ionomycin for 4-5 hours before assessment of the of IL-17 expression. Histograms show intracellular expression of IL-17; DYNABEADS® CD3/ICOS (ISA-3), CD3 high and low (1.5 and 0.06), DYNABEADS® CD3/ICOS (different ICOS C398.4A purchased from eBiocienses, Affymetrix), low CD3 (0.06), and DYNABEADS® CD3/CD28 CTS™. The activation signal strength and the nature of costimulation highly influence polarization and expansion of Th17 cells. Note: Activation with DYNABEADS® CD3/ICOS (mid-0.3) result in a similar phenotype as “(high-1.5)”, not shown.

Successful polarization and expansion of Th17 cells is only achieved by activation of the T cells with DYNABEADS® CD3/ICOS (ISA-3 clone) designed to provide a weak CD3 signal (CD3-0.06). Only the ISA-3 ICOS clone is functional in this study.

DYNABEADS® CD3/CD28 CTS™ (CD3-1.5) result in low/no Th17 polarization/expansion.

### Example 9

### Effects of Bead:Cell Ratio on Selective Expansion of IL-17 Producing Cells

T cells are harvested from three donors (A, B and C). PBMC collected from healthy donors by leukapheresis and further purified on Ficoll-sodium metrizoate density gradients. Astarte Biologics (Normal PBMC). CD3+ T cell enrichment by using Dynabeads® Untouched™ Human T Cells Kit (Thermo Fisher Scientific Cat no. #11344D) according to manual provided.

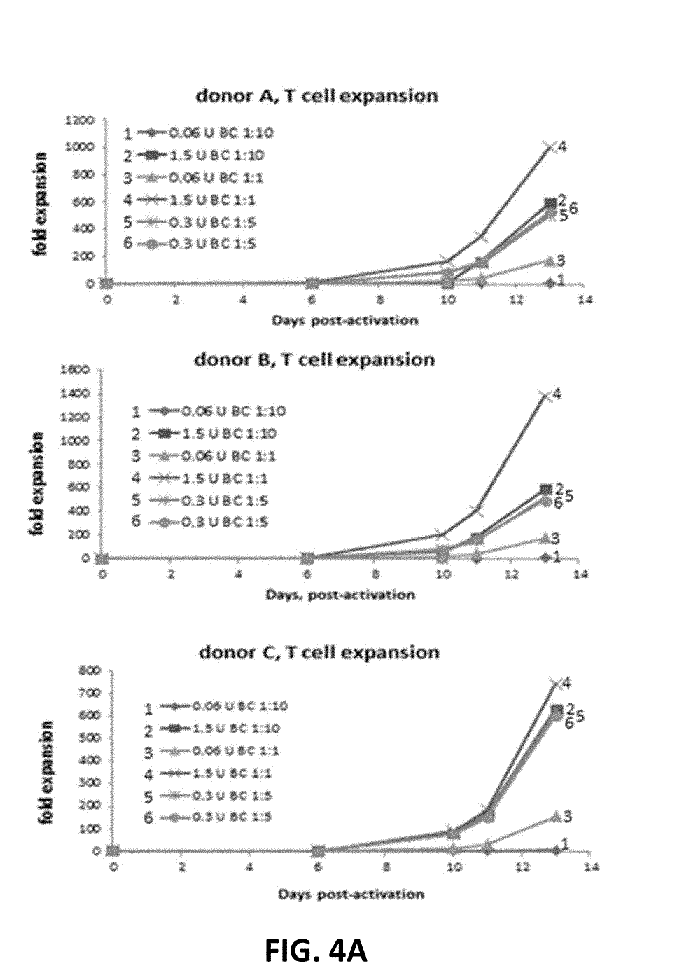

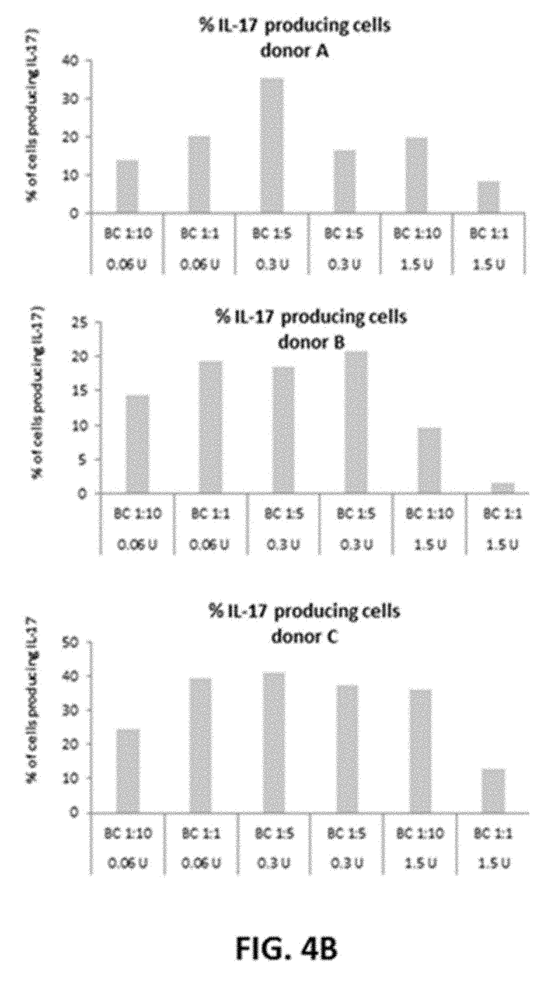

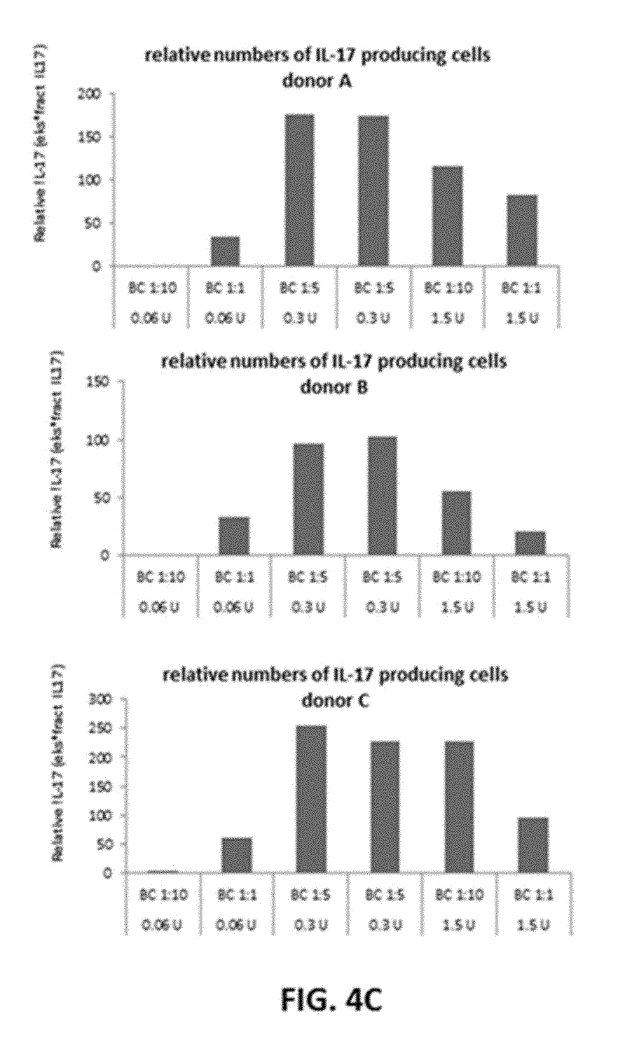

Negatively isolated CD3+ T cells are stimulated with DYNABEADS® CD3/ICOS (low-mid-high) at given bead:cell ratios (BC 1:1, 1:3, 1:5) (Table 4) and cultured for 3 days with Th17 polarizing cytokines. TGF-β1 (Thermo Fisher Scientific, PHG9214; 10 ng/ml, IL-10: PHC0816 10 ng/ml, IL-23; PHC9324, 20 ng/ml, rIL-6; PHC0066 10 ng/ml, and IL-2 (100 ULml, from day 3. Anti-IFNγ antibody AHC4032 10 μg/ml, Anti human IL4 from E. Bioscience (cat. no. BMS129) 10 μg/ml. Split as required. At day 10, total expansion measured (T cell expansion) and cells are restimulated and fraction of IL-17 producing cell is assessed by intracellular flow cytometry staining for IL-17 (% IL-17 producing cells). Data shown in FIG. 4A through FIG. 4C indicate the effects of bead:cell ratio on selective expansion of Th17 cells. Relative numbers of IL-17 producing cells are reported as the fraction of IL-17 producing cells multiplied by total fold T cell expansion.

TABLE 4

| | | | Batch T-cell | Fraction of T | Relative expansion |
|---|---|---|---|---|---|
| Donor | AB | BC | expansion | cells IL-17+ (%) | of IL-17+ T cells |
| A | 1:50 | 1:10 | 4 | 13.7 | 0.4 |
| A | 1:50 | 1:1 | 171 | 20.1 | 34 |
| A | 1:10 | 1:5 | 498 | 35.3 | 176 |
| A | 1:10 | 1:5 | 526 | 16.4 | 174 |
| A | 1:1 | 1:10 | 590 | 19.7 | 116 |
| A | 1:1 | 1:1 | 997 | 8.2 | 82 |
| B | 1:50 | 1:10 | 7 | 14.4 | 1 |
| B | 1:50 | 1:1 | 171 | 19.3 | 33 |
| B | 1:10 | 1:5 | 522 | 18.4 | 96 |
| B | 1:10 | 1:5 | 492 | 20.8 | 102 |
| B | 1:1 | 1:10 | 589 | 9.5 | 56 |
| B | 1:1 | 1:1 | 1377 | 1.5 | 21 |
| C | 1:50 | 1:10 | 7 | 24.6 | 2 |
| C | 1:50 | 1:1 | 155 | 39.2 | 61 |
| C | 1:10 | 1:5 | 623 | 41.0 | 256 |
| C | 1:10 | 1:5 | 604 | 37.5 | 226 |
| C | 1:1 | 1:10 | 632 | 36.1 | 228 |
| C | 1:1 | 1:1 | 744 | 12.7 | 95 |

AB = CD3/ICOS antibody ratio
BC = Bead to cell ratio

### Example 10

### Protocols to Generate Th17 Cells

Introduction

T cells secreting IL-17A (Th17 and Tc17 cells) regress tumors to a greater extent than non-polarized T cells or IFN-γ secreting Th1 or Tc1 cells following adoptive transfer into tumor-bearing mice (Muranski et al., Blood, 112:362-73, (2008), Nelson et al, J Immunol, 194:1737-47 (2015), Guedan et al., Blood, 124:1070-80 (2014)). The factors that modulate the generation of human Th17 cell development remain a matter of debate and differentiation of Th17 cells has been reported to be controlled by various transcription factors, including RORγt, IRF4, RUNX1, BATF, and STAT3 (Nalbant and Eskier, Front. Biosci. (Elite Ed.), 8:427-35 (2016)). Identification of robust culture conditions that can expand Th17 cells for adoptive cell transfer is desirable. DYNABEADS®CD3/CD28 CTS™ activation yields a T-cell product characterized by a Th1 polarized phenotype, while a weaker CD3-signal provided to the T cells under certain polarizing conditions induces Th17 development (Purvis et al., Blood, 11:4829-37 (2010)). It has been found that an optimized CD3 signal strength can be achieved by lowering the amount of agonistic CD3 antibody conjugated to beads together with a low bead to cell ratio of 1:5. It has also been found that the weak CD3 signal together with ICOS costimulation generates a higher fraction of IL-17A producing T cells compared to beads providing CD28 costimulation.

Alternative pathways and protocols demonstrated to generate Th17 cells have been reported: i) CD5 or CD6 costimulation in presence of IL-1β, IL-6, IL-23 and TGF-β (de Wit et al., Blood, 118:6107-14 (2011)), ii) engagement of SLAMF3 and SLAMF6 along with Ag-mediated CD3/TCR stimulation (Chatterjee et al., J Immunol, 188:1206-12 (2012)), iii) agonists acting on aryl hydrocarbon receptor (ARH) (Veldhoen et al., J Exp Med, 206:43-9 (2009), and iv) cholesterol precursors which functions as potent retinoic acid receptor-related orphan receptors (ROR) agonists (Hu et al., Nat Chem Biol, 11:141-147 (2015)). However, the commonly used medium RPMI supports only low levels of Th17 polarization. Other media richer in aromatic amino acids, precursors of arly hydrocarbon receptor (AHR) agonists, consistently results in higher Th17 expansion and demonstrate that AHR activation is essential Th17 polarization. RORγt is the master transcription factor for Th17 cells. When injected into mice, RORγt agonist result in elevated levels of Th17 cytokines, increase the level of costimulatory receptors such as CD137, and decrease expression of the co-inhibitory receptor PD-1 (Hu et al., “RORg agonist regulate immune checkpoint receptors to enhance anti-tumor immunity” AACR abstract #565, New Orleans 2016). Furthermore, T cells treated with RORγt agonist in vitro maintained low PD-1 expression following adoptive transfer.

Significant Th17 polarization of T cells following low CD3 strength activation and CD5 costimulation under polarizing conditions, including the use of AHR agonists, has been found.

Materials and Methods

Primary T cells and culturing conditions: Peripheral blood mononuclear cells (PBMCs) were isolated from healthy donor buffy coats or aphaereses (Astarte Biologics WA, USA) under informed consent. Negatively isolated T cells (DYNABEADS® Untouched Human T Cells, cat. no. 11344D, Thermo Fisher Scientific, Waltham, Mass.) were activated by using DYNABEADS® with various ligand compositions and stoichiometry as described. Polarizing conditions are described in the text. Activated T cells were cultured at 37° C. and 5% CO2 in X-VIVO15™ (cat. no. BE02-060F, Lonza Biologics, MD, USA), plus 2-5% CTS™ Immune Cell SR (cat. no. A25961-02, Thermo Fisher Scientific, Waltham, Mass.) and 0.25 μg/mL Gentamicin (cat. no. 15750060, Thermo Fisher Scientific) and expansion was achieved by adding cytokines and fresh medium every 1-3 days to maintain a cell concentration of 0.5-2×10⁶cells/mL as specified.

Generic protocol: Th17/Tc17 cells were expanded in medium containing polarizing cytokines (IL-6, IL-1β, IL-23, and TGF-β (cat. nos. IL6—PHC066, IL-1β—PHC0816, IL23-PHC9324, and TGF-β13 PHG9214, Thermo Fisher Scientific, Waltham, Mass. in presence of anti-IL-4 and anti-IFN-γ neutralizing antibodies (cat. nos. AC0642 and ACH4032, Thermo Fisher Scientific, Waltham, Mass.) as described in Paulos et al., Sci Transl Med, 2:55ra78, (2010). Polarizing cytokines and antibodies were only added for the initial activation (day 0-3). Cells were split and maintained in medium containing 100 IU IL-2/mL and IL-23 from day 3 post activation.

Stimulatory Dynabeads: The relative amounts of stimulatory anti-CD3 antibody and its co-conjugated costimulatory ligand conjugated to Dynospheres are summarized in Table 5.

TABLE 5
Dynabeads Th17 expander prototypes and controls

| Dynabeads conjugation | CD3 (XR-CD3) conc. | Costimulation |
|---|---|---|
| Dynabeads ®CD3/ICOS (mid) | 0.3 | ICOS |
| Dynabeads ®CD3/CD5 (mid) | 0.3 | CD5 |
| Dynabeads ®CD3/CD28 (mid) | 0.3 | CD28 |

Anti-CD3 and anti-CD28 antibodies XR-CD3 and XR-CD28 (Thermo Fisher Scientific, Norway), Anti-ICOS antibody ISA-3 (Affymetrix/eBioScience CA USA), Anti-CD5 antibody clone UCHT2 (Affymetrix/eBioScience CA. USA).

Polarization and expansion of Th17 cells. T cells isolated from healthy donor PBMC by negative isolation (DYNABEADS® Untouched Human T Cells, cat. no. 11344D, Thermo Fisher Scientific, Waltham, Mass.) were activated with DYNABEADS®-Th17 prototypes (bead to cell ratio 1:5) designed to deliver different costimulatory signals in presence of polarizing cytokines (IL-1β, IL-21, IL-23, IL-6, TGF-β) and neutralizing IL-4 and IFNγ antibodies (Table 5). Activated T cells were left undisturbed for 2-3 days and thereafter expanded for 10-14 days by adding fresh culture media and cytokines with IL-2 and IL-23. T cell expansion was assessed by cell counting (Coulter Counter, Beckman Coulter, CA USA). At day 10-14 of expansion, cells were stimulated with PMA/Ionomycin for 5 hours in medium containing monensin (BD GOLGISTOP™, cat. no. 554724, BD Biosciences) and brefeldin A (BD GOLGIPLUG™, cat. no. 555029, BD Biosciences) before being analyzed by flow cytometry for phenotype markers and intracellular expression of IL-17A and IFNγ after fixation and permeabilization.

TABLE 6
Polarizing agents and their function

| Polarizing agent | Activity on T cells |
|---|---|
| IL-1β | IL-1β drives Th17 polarization in a variety |
| | of inflammatory conditions. IL-1 signaling |
| | promotes proliferation and survival of antigen- |
| | stimulated helper T cells. IL-1 also induces IL-21 |
| | autocrine signaling loop via activation of Irf4- |
| | transcriptional network. Irf4-deficient animals |
| | have increased numbers of Foxp3+ Tregs and a |
| | diminished ability to form Th17 responses despite |
| | intact Stat3 signaling. |
| IL-23 | IL-23 stabilizes IL-17 expression, increases IL-17 |
| | production but typically is not alone sufficient to |
| | induce Th17 differentiation. IL-23 deficient mice |
| | contain very few Th17 cells and are protected |
| | from certain autoimmune diseases. |
| TGF-β | TGF-β central role—drives Th17 polarization |
| | in combination with IL-1β or IL-21, and induces |
| | Treg generation alone. Low concentration of |
| | TGF-β favors, while high concentration |
| | inhibits Th17 generation. |
| IL-6 | IL-6 in combination with TGF-β drives |
| | differentiation from naïve T cells |
| | (absence of IL-6 generates Tregs). |
| IL-21 | IL-21 (via Stat3 signaling) and TGF-β are |
| | sufficient to induce RoR-γt and IL-23R |
| | expression and drive robust Th17 polarization |
| | in the absence of IL-6. In presence of IL-6, |
| | stimulated CD4+ T cells produce IL-21 |
| | themselves, which further augments its secretion |
| | in an autocrine self-amplifying loop. |
| IL-2 | T cell growth factor |
| (from day 3) | |
| Anti-IL-4 | Block Th2 differentiation |
| antibody | |
| Anti-IFNγ | Block Th1 differentiation |
| antibody | |

Flow cytometry, phenotype and functionality: Intracellular cytokine expression in PMA/Ionomycin (Cell Stimulation Cocktail Kit containing protein transport inhibitors Brefeldin and Monensin, cat. no. 00-4971, eBioscience) activated Th17 cells were detected using IL-17A-PE (cat no. A18695, clone: 4H1524, Molecular Probes) and IL17-F, IFN-γ and appropriate isotype controls (IgG2b PE, Cat No. 400313 BioLegend). Flow cytometric data were collected on a BD LSRII (BD Biosciences) and analyzed with FACS Diva software (BD Biosciences).

Results and Discussion

i). Effect of Neutralizing Antibodies

αIL-4/α-IFNγ antibodies were utilized in Th17 cell polarization protocols to block Th1/Th2 polarization.

Blocking antibodies were introduced to improve Th17 generation after CD3/CD28 activation. Clinical grade blocking antibodies are expensive and add complexity to the Th17 cell generation protocol.

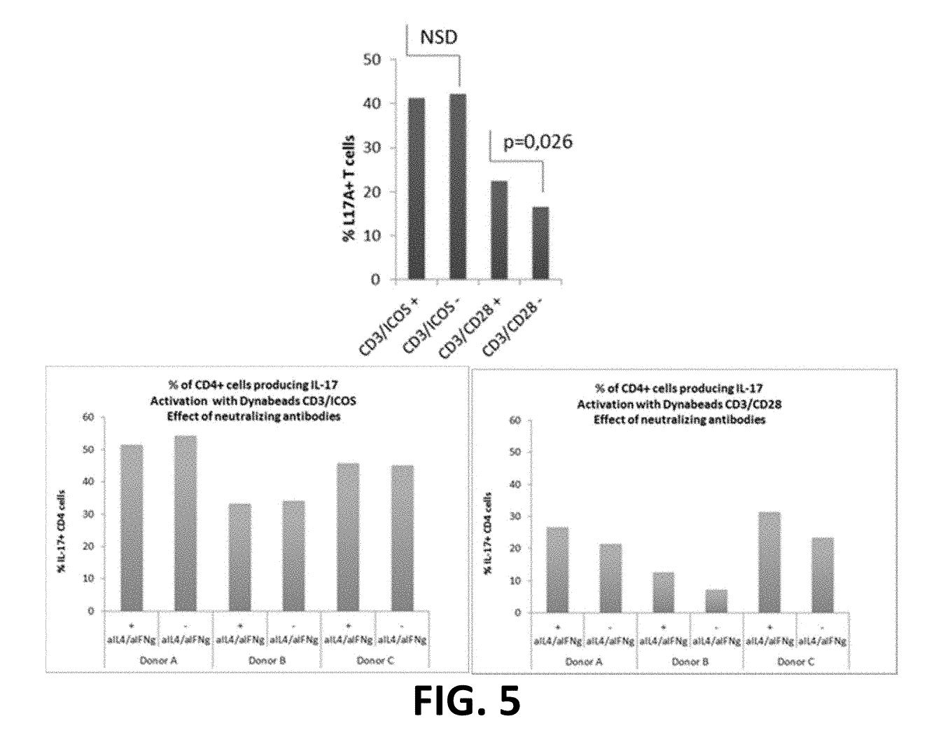

Protocol permutation: T cells are activated using optimized conditions with either DYNABEADS® CD3/ICOS or DYNABEADS® CD3/CD28. Th17 polarization is facilitated by cytokines TGF-β, IL-23, IL-6, IL-1β. The effect of αIL-4/α-TN-γ neutralizing antibodies on the fraction of IL-17 producing CD4+ cells post-expansion are compared in FIG. 5.

Conclusion: Stimulation through CD3/ICOS results in higher fraction IL-17 producing T cells compared to CD3/CD28 (42.5 vs. 16.5% of CD4 cells). Generation of Th17 cells after stimulation of CD4+ T cells with DYNABEADS® CD3/ICOS (+Th17 polarizing cytokines) is less critically dependent on neutralizing IFN-γ and IL-4 antibodies. Stimulation with DYNABEADS®s CD3/CD28 requires addition of Th1/Th2 blocking antibodies.

ii). Effect of CD5 Costimulation

CD5 has been reported to promote Th17 development through elevation of IL-23R expression, resulting in prolonged STAT3 activation and enhanced levels of ROR-γt compared to CD28 costimulation (de Wit et al., Blood, 118: 6107-14 (2011)).

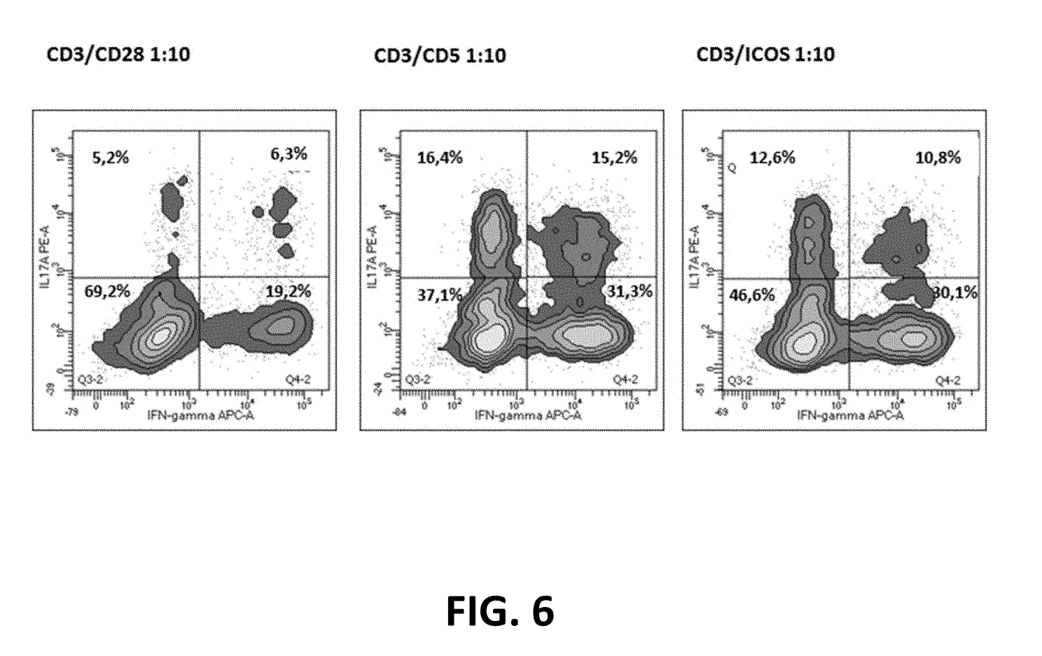

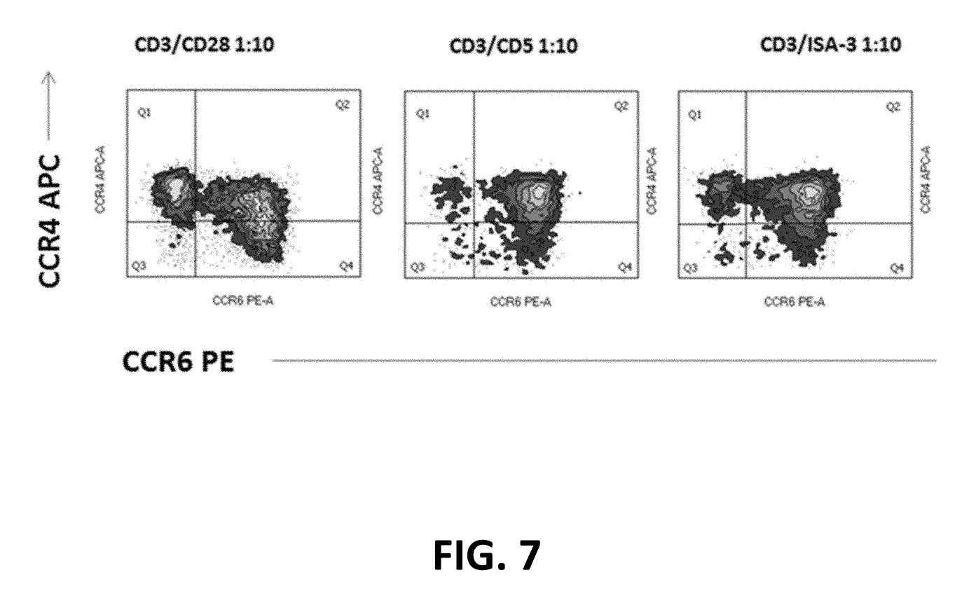

Protocol permutation: T cells were activated with DYNABEADS® CD3/CD5, DYNABEADS® CD3/ICOS, or DYNABEADS® CD3/CD28 with equal signal strength and stoichiometry. Th17 polarization was facilitated by addition of cytokines (TGF-β, IL-23, IL-6, IL-1β) and αIL-4/α-TN-γ neutralizing antibodies. The fractions of IL-17 and IFN-γ producing CD4+ T cells (intracellular staining), as well as the fractions of CCR4+CCR6+ expressing cells—a phenotype associated with Th17—were compared (FIG. 6 and FIG. 7).

Conclusion: Stimulation through CD3/CD5 yields a comparable to or slightly higher Th17 profile compared to CD3/ICOS, and is superior to CD3/CD28 stimulation, as determined by IL-17 production and CCR4/CCR6 co-expression.

iii). Effect of AHR Agonists

The AHR ligand FICZ (5,11-Dihydroindolo(3,2-b)carbazole-6-carboxaldehyde) has been shown to up-regulate the Th17 program (Veldhoen et al., J. Exp. Med., 206:43-9 (2009).

Protocol permutation: T cells were activated with DYNABEADS® CD3/CD5, DYNABEADS® CD3/ICOS, or DYNABEADS® CD3/CD28 with equal signal strength and stoichiometry. Th17 polarization was facilitated by addition of i) cytokines (TGF-β, IL-23, IL-6, IL-1β) and αIL-4/α-TN-γ neutralizing antibodies or ii) the AHR agonist FICZ plus IL-6, IL-1βa. Th17 polarization was assessed by intracellular staining of IL-17 and IFN-γ following PMA/Ionomycin activation of polarized T cells (FIG. 8).

Conclusion: FICZ had promising activity on T cells costimulated through CD5 but not ICOS. The FICZ, IL-1β and IL-6 protocol generates fewer IL-17 producing cells compared to standard polarizing conditions, and optimization is required.

### Example 11

### Expansion of Treg Cells (>80% FOXP3) with DYNABEADS™ Treg Beads

TABLE 7
Materials Used in this Example and Material Sources

| Reagents | Source/Cat. No. |
|---|---|
| Treg Cells | Flow sorted, FOXP3 > 80% |
| DYNABEADS ™ Treg beads | Different Prototypes |
| Control beads; | Thermo Fisher: 40203D |
| DYNABEADS ™CD3/CD28 CTS | |
| Control beads; MACS GMP | Miltenyi: 170-076-119 |
| ExpAct Treg Kit | |
| XVIVO ™ 15 | Lonza: BE02-060F |
| CTS Immune Cell Serum | Thermo Fisher: A2596102 |
| Replacement | |
| rIL-2 | Thermo Fisher: PHC0023 |

Sorted Treg cells (>80% FOXP3) were diluted in X-Vivo 15+5% CTS Immune Cell Serum Replacement (X-VIVO™ 15/CTS) to 2 million cells/mL. DYNABEADS™ were washed once by magnetic isolation and diluted the beads to 4 million/mL in X-VIVO™ 15/CTS. Control beads were washed once and dilute the beads to 4 million/mL in X-VIVO™ 15/CTS with 300 U/mL rIL2. Equal amounts of cells, X-VIVO™ 15/CTS, and beads were then added wells of a 48-well plate to a total volume of 400 μl. Setup was done in triplicate for each sample.

TABLE 8
Reagent Mixtures

| | | | Total | X-VIVO ™ | 4 million | 2 million |
|---|---|---|---|---|---|---|
| | | | volume | 15/CTS | beads/mL | cells/mL |
| No | Beads:cells | Beads | (μL) | (μL) | (μL) | (μL) |
| 1 | 4:1 | DYNABEADS ™ Treg bead | 400 | 100 | 200 | 100 |
| 2 | 4:1 | Control (DYNABEADS ™CD3/CD28 CTS) | 400 | 100 | 200 | 100 |
| 3 | 4:1 | Control (MACS GMP ExpAct Treg Kit) | 400 | 100 | 200 | 100 |

Day 0: Treg expansion mixtures were prepared in 48-well plate with X-VIVO™ 15/CTS with 300 U/mL rIL2, as above.

Day 2: 200 μL X-VIVO™ 15/CTS and 300 U/mL rIL2 were added to each wells and the resulting cell suspension was mixed.

Days 3-12: The cell density was estimated each day by microscope. When a density of approximately 1.5-2 million cells/cm² was reached, the cells are transferred to larger wells or flasks. Fresh X-VIVO™ 15/CTS with 300 U/mL rIL2 was added as needed.

Day 5: 250 μL X-VIVO™ 15/CTS was removed from each sample and 250 μL fresh X-VIVO™ 15/CTS with 300 U/mL rIL2 was added. The cell suspension was then mixed.

Day 7: Well volume and cells counts were determined. When cell densities of approximately 1.5-2 million cells/cm² were reached, the cells were transferred to bigger wells/flasks and fresh X-VIVO™ 15/CTS with 300 U/mL rIL2 was added.

Day 9: Fresh X-VIVO™ 15/CTS with 300 U/mL rIL2 was again added and the cell suspension was mixed.

Day 12: The volume was determined in each well and cells were counted. 100,000-200,000 cells were used for FoxP3 staining using reagents set out in Table 9.

TABLE 9

| Staining colors | Clone | Source/Cat. No. |
|---|---|---|
| CD4 PerCP | S3.5 (IgG2a) | Thermo Fisher, MHCD0431 |
| CD25 APC | CD25-3G10 | Thermo Fisher, MHCD2505, |
| | (IgG1) | |
| CD127 PE | R34-34 | Thermo Fisher, A18684 |
| | (IgG1 kappa) | |
| FOXP3 Alexa Fluor 488 | 259D/C7 | BD Biosciences, 560047 |
| | (IgG1) | |
| Fixation/Permeabilization | N.A. | eBioscience, 005123-43 |
| concentrate (4x) | | |
| Fixation/Permeabilization | N.A. | eBioscience, 00-5223-56 |
| diluent | | |
| Permeabilization | N.A. | eBioscience, 008333-56 |
| concentrate (10x) | | |

Fixation/permeabilization solution was prepared by mixing Fixation/Permeabilization (1 part) with Fixation Permeabilization diluent (3 parts). Fixation/permeabilization buffer/wash was prepared by mixing Permeabilization concentration (1 part) with water (9 parts). Prepared fresh each day. Typically, 0.1-0.2 million cells were stained.

The cells were washed once with 1 mL DPBS/0.1% HAS and centrifuged a 350×g, 8 minutes to remove the supernatant.

Pulse vortex the cells were then mixed by vortexing. One mL of fixation/permeabilization (1×) solution was added and the cells were vortexed again. The cell suspension was then incubated for 60 minutes at 4° C. Two mL permeabilization buffer/wash (1×) was then added and the suspension was centrifugation at 400×g for 8 minutes at 4° C. to remove the supernatant. Addition of permeabilization buffer/wash followed by centrifugation was repeated once.

Staining mixture 50 μL (2.5 μL CD4 PerCP, 2.5 μL CD25 APC, 5 μL FOXP3 AF488, 2 μL CD127PE+38 μL permeabilization buffer/wash (1×)) was then added to the samples, followed by incubation for 30 minutes at 4° C. The samples were then analyzed by flow cytometry.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference.

While this invention has been particularly shown and described with references to specific embodiments, it will be understood by those skilled in the art that various changes in form and details may be made therein without departing from the scope of the invention encompassed by the appended claims.

Exemplary Subject Matter of the Invention is Represented by the Following Clauses:

Clause 1. A method for selectively expanding members of a T cell subpopulation, the method comprising exposing a mixed population of T cells to:

(a) a first agent which provides a primary activation signal to the members of the T cell subpopulation, thereby activating the T cells, and

(b) a second agent and a third agent, each of which stimulates two or more different accessory molecules on the members of the T cell subpopulation, thereby stimulating the proliferation of the activated T cells of (a), wherein the ratios of the first agent, the second agent, and the third agent are adjusted to induce the members the T cell subpopulation to selectively expand over members of other T cell subpopulations.

Clause 2. The method of clause 1, wherein the first agent is an anti-CD3 antibody.

Clause 3. The method of clause 1 or 2, wherein the second agent is an antibody.

Clause 4. The method of any one of the previous clauses, wherein the third agent is an antibody or a non-antibody protein.

Clause 5. The method of any one of the previous clauses, wherein the non-antibody protein is a chemokine or cytokine.

Clause 6. The method of clause 4, wherein the chemokine or cytokine is one or more protein selected from the group consisting of:

(a) Interleukin-1α,

(b) Interleukin-2,

(c) Interleukin-4,

(d) Interleukin-1β,

(e) Interleukin-6,

(f) Interleukin-12,

(g) Interleukin-15,

(h) Interleukin-18,

(i) Interleukin-21,

(j) Interleukin-7,

(k) Interleukin-23, and

(l) Transforming growth factor β1.

Clause 7. The method of any one of the previous clauses, wherein the ratio of the first agent, the second agent, and the third agent are adjusted such that the first agent is lower in concentration compared to the second or third agent.

Clause 8. The method of clause 7, wherein the lower concentration of the first agent is about 0.34 units.

Clause 9. The method of clause 8, wherein the first agent is an anti-CD3 antibody at a concentration of about 0.34 units, and a second agent is an anti-CD28 antibody at a concentration of 3.4 units.

Clause 10. The method of any one of the previous clauses, wherein the T cell subpopulation selectively expanded are Treg cells.

Clause 11. The method of any one of the previous clauses, wherein the lower concentration of the first agent is about 0.01 units.

Clause 12. The method of any one of the previous clauses, wherein the first agent is an anti-CD3 antibody at a concentration of about 0.01 units, and a second agent is an anti-CD28 antibody, and a third agent is an anti-CD137 antibody.

Clause 13. The method of any one of the previous clauses, wherein the T cell subpopulation selectively expanded are memory T cells.

Clause 14. The method of any one of the previous clauses, wherein the lower concentration of the first agent is about 0.06 units.

Clause 15. The method of any one of the previous clauses, wherein the first agent is an anti-CD3 antibody at a concentration of about 0.34 units, and a second agent is an anti-ICOS or anti-CD5 antibody.

Clause 16. The method of any one of the previous clauses, wherein the T cell subpopulation selectively expanded are Th17 cells.

Clause 17. A method for selectively expanding T cell subpopulations, said method comprising

(a) exposing T cells to CD3, CD28, anti-CD5, anti-ICOS and/or CD137 signals ex vivo, and

(b) culturing said T cells in a manner that allows for the expansion of Th17 cells, antigen experienced T cells and/or regulatory T cells.

Clause 18. The method of clause 17, wherein the CD3, CD28 and CD137 signals are mediated by an anti-CD3, an anti-CD28 and/or an anti-CD137 antibodies.

Clause 19. The method of clause 17 or 18, wherein the anti-CD3, the anti-CD28 and the anti-CD137 antibodies are used in a range that encompasses the concentrations of 0.01 units to 1.5 units for the expansion of memory T cells.

Clause 20. The method of clause 17 or 18, wherein the anti-CD3, the anti-CD28 anti-CD5, anti-ICOS and the anti-CD137 antibodies are used in a range that encompasses the concentrations of 0.06 units to 1.5 units for the expansion of Th17 cells.

Clause 21. The method of clause 17 or 18, wherein the anti-CD3, the anti-CD28 and the anti-CD137 antibodies are used in a range that encompasses the concentrations of about 0.34 units to 3.41 units for the expansion of Treg cells.

Clause 22. The method of any one of clauses 17 to 21, wherein the anti-CD3 antibodies are used in a lower concentration compared to the concentration of the anti-CD28 and the anti-CD137 antibodies.

Clause 23. The method of any one of clauses 17 to 22, wherein the T cells are isolated using CD3 selection.

Clause 24. The method of any one of clauses 17 to 23, wherein the Th17 cells are CD3+, CD8/CD4+/, and produces IL-17 cytokine.

Clause 25. The method of any one of clauses 17 to 24, wherein the Th17 cells are capable of producing IL-17, IL-21 and/or IL-22.

Clause 26. The method of any one of clauses 17 to 25, wherein the memory T cells are selected from the group consisting of stem memory T cells, central memory T cells, and effector memory T cells.

Clause 27. The method of clause 26, wherein the stem memory cells have one or more of following markers: CD3+, CD45RO−, CCR7+, CD45RA+, CD62L+ (L-selectin), CD27+, CD28+, IL-7Ra+, IL-2Rβ, CXCR3, and LFA-1.

Clause 28. The method of clause 26 wherein the central memory cells have one or more of following markers: CD3+, CCR7+, CD45RA−, CD45RO+, CD62L+ (L-selectin), CD27+, and CD28+.

Clause 29. The method of clause 28, wherein the central memory cells are capable of producing IL-2.

Clause 30. The method of clause 26, wherein the effector memory cells have one or more of following markers: CD28+/−, CD27+/−, CD3+, CD4+, CD8+, CCR7−, CD45RA−, CD45RO+.

Clause 31. The method of clauses 26 or 30, wherein the effector memory cells are cells are capable of producing IFNγ and IL-4.

Clause 32. The method of any one of clauses 17 to 24, wherein the Treg cells have one or more of following markers: CD4+, CD25+, FOXP3+ and CD127neg/low.

Clause 33. A method for selectively expanding T regulatory cells, said method comprising:

(a) exposing T cells to CD3 and CD28 signals ex vivo, and

(b) culturing said T cells in a manner that allows for the expansion of T regulatory cells.

Clause 34. The method of clause 33, wherein the CD3 and the CD28 signals are mediated by anti-CD3, and anti-CD28 antibodies.

Clause 35. The method of clause 34, wherein the anti-CD3, and anti-CD28 antibodies are used in a range that encompasses concentration range of the 0.34 to 3.4 units.

Clause 36. The method of clause 34 or 35, wherein the anti-CD3 antibodies are used in a lower concentration compared to the concentration of anti-CD28 and/or antibodies.

Clause 37. A method for selectively expanding Th17 cells, said method comprising:

(a) exposing CD3+ T cells to CD3, CD28, anti-CD5, and/or ICOS signals ex vivo, and

(b) culturing said CD3+ T cells in a manner that allows for the expansion of Th17 cells,

wherein the amount of CD3, CD28, CD5 and/or ICOS are not the same.

Clause 38. The method of clause 37, wherein the CD3, CD28, CD5, and ICOS signals are mediated by anti-CD3, anti-CD28, anti-CD5, and anti-ICOS antibodies.

Clause 39. The method of clause 37 or 38, wherein the anti-CD3, anti-CD28, anti-CD5, and anti-ICOS antibodies are used in a range that encompasses the concentration range of 0.06 to 1.5 units for the expansion of Th17 cells.

Clause 40. The method of any one of clauses 37, 28 or 39, wherein the anti-CD3 antibodies are used in a lower concentration compared to the concentration of anti-CD28, anti-CD5, and/or anti-ICOS antibodies.

Clause 41. A method for selectively expanding antigen experienced T cells, said method comprising:

(a) exposing T cells to CD3, CD28, CD27 and/or CD137 signals ex vivo, and

(b) culturing said T cells in a manner that allows for the expansion of antigen experienced T cells.

Clause 42. The method of clause 41, wherein the CD3, CD28 and CD27 and/or anti-CD137 signals are provided by anti-CD3, anti-CD28, anti-CD27 and/or anti-CD137 antibodies.

Clause 43. The method of clause 41 or 42, wherein the anti-CD3, anti-CD28, anti-CD27 and anti-CD137 antibodies are used in a range that encompasses the concentration range 0.01-1.5 units.

Clause 44. The method of any one of clauses 41, 42 or 43, wherein the anti-CD3 antibodies are used in a lower concentration compared to the concentration of anti-CD28 and/or anti-CD137 antibodies.

Clause 45. A composition comprising CD3+ (1) T cells and (2) beads containing (a) anti-CD3 antibodies and (b) anti-CD28, anti-CD137 or ICOS antibodies capable of selective expansion of T cell subpopulations, wherein the amount of (a) anti-CD3 antibodies and (b) anti-CD28, anti-CD137 or ICOS antibodies present are not the same.

Clause 46. The composition of clause 45, wherein the T cell subpopulation is selected from the group consisting of Th17 cells, antigen experienced T cells and/or regulatory T cells.

Clause 47. The composition of clause 45 or 46, wherein anti-CD3, anti-CD28, anti-CD27 and anti-CD 137 antibodies are used in a range that encompasses the concentration range of 0.01 to 1.5 units for the expansion of memory T cells.

Clause 48. The composition of any one of clauses 45, 46 or 47, wherein the anti-CD3, anti-CD28 and anti-CD137 antibodies are used in a range that encompasses the concentration range of 0.06 to 1.5 units for the expansion of Th17 cells.

Clause 49. The composition of any one of clauses 45 to 48, wherein the anti-CD3, and anti-CD28 antibodies are used in a range that encompasses the concentration range of 0.34 to 3.41 units for the expansion of Treg cells.

Clause 50. The composition of any one of clauses 45 to 49, wherein the anti-CD3 antibodies are used in a lower concentration compared to the concentration of anti-CD28 and anti-CD137 antibodies.

Clause 51. A composition comprising (1) T cells and (2) beads containing anti-CD3, anti-CDS, anti-CD28, anti-ICOS, and anti-CD137 antibodies capable of selective expansion of Th17 cells, wherein the Th17 cells are capable of producing one or more effector cytokines and wherein the amount of anti-CD3 and anti-CD5/ICOS/CD28/CD137 antibodies present on the beads are not the same.

Clause 52. The composition of clause 51, wherein the one or more effector cytokine is selected from the group consisting of IL-17, IL-21, and IL-22.

Clause 53. A composition comprising (1) T cells and (2) beads containing anti-CD3, anti-CD28, and anti-CD137 antibodies capable of selective expansion of antigen experienced memory T cells, wherein the T cells are capable of recognizing specific antigen and, wherein the amount of anti-CD3 and anti-CD28 antibodies present on the beads are not the same.

Clause 54. The composition of clause 53, wherein the specific antigen is selected from the group consisting of viral antigens, bacterial, fungal, protozoal, and cancer antigens.

Clause 55. The composition of clause 54, wherein the viral antigen is selected from a group consisting of CMV, EBV, Influenza, and HIV.

Clause 56. The composition of clause 53, wherein the antigen is selected from a group consisting of Streptococci M-protein, Neisseria pilli, Borrelia burgdorferi lipoprotein VisE, B. pseudomallei polysaccharide antigens, Aspergillus fumigatus galactomannan, and F. tularensis lipopolysaccharide.

Clause 57. A composition comprising (1) T cells and (2) beads containing anti-CD3 and anti-CD28 antibodies that are capable of selectively expanding regulatory T cells, wherein the amount of anti-CD3 and anti-CD28 antibodies present on the beads are not the same.

Clause 58. The composition of clause 57, wherein the regulatory T cells are CD4+ CD25+ FOXP3+ CD127low/neg.

Clause 59. The composition of clause 57 or 58, wherein the regulatory T cells activity comprises suppressive activity.

Clause 60. A method of treating an individual in need thereof, said method comprising administering to the individual a pharmaceutically acceptable composition comprising Th17 cells, antigen experienced T cells, and/or regulatory T cells.

Clause 61. The method of clause 60, wherein the individual in need thereof is affected by cancer, inflammatory diseases, autoimmune diseases, allergic disease, or infectious diseases.

Clause 62. The method of clause 61, wherein the cancer is lung, ovarian, pancreatic, breast, liver and skin cancer.

Clause 63. The method of clause 61, wherein the inflammatory disease is selected from the group consisting of diabetes; rheumatoid arthritis; inflammatory bowel disease; familial mediterranean fever; neonatal onset multisystem inflammatory disease; tumor necrosis factor (TNF) receptor-associated periodic syndrom (TRAPS); deficiency of interleukin-1 receptor antagonist (DIRA); Systemic Lupus; Uveitis; and Behcet's disease.

Clause 64. A method of reconstituting an immune system of an individual in need thereof, said method comprising administering to the individual a pharmaceutically acceptable composition comprising Th17 cells, antigen experienced T cells, and/or regulatory T cells.

Clause 65. A method of providing adoptive immunotherapy to an individual in need, said method comprising administering to the individual a pharmaceutically acceptable composition comprising Th17 cells, antigen experienced T cells, and/or regulatory T cells.

Clause 66. The method according to any one of clauses 60-65, wherein the T cells are genetically modified.

Clause 67. The method according to clause 66, wherein the genetic modification is chimeric antigen receptor.

Clause 68. The method according to clause 66, wherein the genetic modification is genetically modified T cell receptor.

Clause 69. A method for selectively altering the proportional ratio of two T cell subtypes in a sample, the method comprising contacting a sample comprising a mixed population of T cells with at least two stimulatory agents, wherein the stimulatory agents provide different amounts of signals to the T cells in the mixed population, wherein one T cell subtype selectively expands as compared to a second T cell subtype.

Clause 70. The method of clause 69, wherein the sample comprises buffy coat cells derived from an individual.

Clause 71. The method of clause 69 or 70, wherein the at least two stimulatory signals stimulate CD3 and CD28 receptors.

Clause 72. The method of clause 69, 70 or 71, wherein at least one T cell subtype is selectively eliminated from the mixed population.

Clause 73. The method of any one of clauses 69 to 72, wherein Treg T cells are increased in proportion with respect to all T cells within the mixed population.

Clause 74. The method of any one of clauses 69 to 73, wherein the total number of memory T cells is decreased in the sample.

Clause 75. The method of any one of clauses 69 to 74, wherein the amount of stimulatory signal CD3 is less than half than the CD28 stimulatory signal.

Clause 76. A method for expanding Th17 cells, the method comprising:

(a) exposing a population of T cells to CD3 and CD5 signals ex vivo, and

(b) culturing the population of T cells under conditions that allows for the expansion of Th17 cells,

wherein the population of T cells is exposed to an aryl hydrocarbon receptor agonist or is not exposed to exogenous Interleukin-23.

Clause 77. The method of clause 76, wherein the population of T cells is a mixed population of different T cells types.

Clause 78. The method of clause 76 or 77, further comprising contacting the population of T cells with one or more polarizing agents.

Clause 79. The method of clause 76, 77 or 78, further comprising contacting wherein the one or more polarizing agents are one or more agent selected from the group consisting of: Interleukin-1β, Interleukin-23, Tumor Growth Factor-β, Interleukin-6, Interleukin-21, Interleukin-2, anti-Interleukin-4 antibody, and anti-Interferon γ antibody.

Clause 80. The method of any one of clauses 76 to 79, wherein the population of T cells is further exposed to an aryl hydrocarbon receptor agonist.

Clause 81. The method of clause 80, wherein the aryl hydrocarbon receptor agonist is 6-formylindolo[3,2-b]carbazole (FICZ).

Clause 82. The method of any one of clauses 69 to 81, wherein the population of T cells is further exposed to Interleukin-10 and Interleukin-6.

Clause 83. The method of any one of clauses 69 to 82, wherein the Th17 cells are engineered to express one or more chimeric antigen receptors.

Clause 84. The method of clause 83, wherein the at least one of the one or more chimeric antigen receptors has specificity for a cell surface antigen of a mammalian cell.

Clause 85. The method of any one of clauses 69 to 84, wherein the cell surface antigen of a mammalian cell is an antigen associated with a tumor cell.

Clause 86. A composition comprising a CD3 signal, a CD5 signal, an aryl hydrocarbon receptor agonist, and one or more cytokine.

Clause 87. The composition of clause 86, wherein the one or more cytokine comprising both Interleukin-10 and Interleukin-6.

Clause 88. The composition of clause 86 or 87, further comprising a population of T cells.

Clause 89. The composition of clause 86, 87 or 89, wherein the CD3 signal is an anti-CD3 antibody.

Clause 90. The composition of any one of clauses 86 to 89, wherein the CD5 signal is an anti-CD5 antibody.

Clause 91. A composition comprising a population of T cells, a CD3 signal, a CD5 signal, an aryl hydrocarbon receptor agonist, and one or more cytokine.

Clause 92. The composition of clause 91, wherein the population of T cells is present in a mixture comprising:

(a) a “buffy coat” sample,

(b) a sample of white blood cells that contains greater than 80% mixed T cells,

(c) a sample that contains greater than 80% CD4+ T cells, or

(d) a sample that contains greater than 80% Th17 cells.

Clause 93. A method for the separation and activation of T cells from a mixed population of cells, the method comprising:

(a) contacting the mixed population of cells with a solid support having bound thereto at least a first ligand with binding affinity for a protein located on T cells present in the mixed population of cells, under conditions that allow for binding of the T cells to the solid support and activation of the same T cells, and

(b) separation of the T cells bound to the solid support from cells not bound to the solid support to obtain a purified T cell population.

Clause 94. The method of clause 93, wherein the solid support has bound thereto at least a first ligand and a second ligand, wherein each of the first ligand and the second ligand have binding affinity for different proteins located on individual T cells present in the mixed population of cells.

Clause 95. The method of clause 94, wherein the first ligand is either an anti-CD3 antibody or an anti-CD4 antibody and wherein the second ligand is a ligand selected from the group consisting of:

(a) an anti-CD5 antibody,

(b) an anti-CD28 antibody,

(c) an anti-CD137 antibody, and

(d) an anti-ICOS antibody.

Clause 96. The method of clause 93, 94 or 95, further comprising releasing cells of the purified T cell population obtained in step (b) from the solid support.

Clause 97. The method of clause 96, further comprising expanding the released T cells.

Clause 98. The method of clause 97, wherein expansion of the released T cells occurs in a culture medium.

Clause 99. The method of clause 98, wherein one or more chemokine or cytokine is present in the culture medium.

Clause 100. The method of any one of clauses 93 to 99, wherein the one or more chemokine or cytokine is present in step (a).

Clause 101. The method of clause 100, wherein the one or more chemokine or cytokine is selected from the group consisting of:

(a) Interleukin-1α,

(b) Interleukin-2,

(c) Interleukin-4,

(d) Interleukin-1β,

(e) Interleukin-6,

(f) Interleukin-12,

(g) Interleukin-15,

(h) Interleukin-18,

(i) Interleukin-21, and

(j) Transforming growth factor β1.

Clause 102. A method for the activation and expansion of T cells, the method comprising contacting a mixed population of T cells with:

(a) a first agent which provides a primary activation signal to the members of a T cell subpopulation by stimulating a molecule on the members of the T cell subpopulation, and

(b) a second agent that stimulates a molecule on the members of the T cell subpopulation that is different than the molecule stimulated by the first agent,

whereby T cells in the population are activated and expand,

wherein the first agent and the second agent are bound to one or more solid supports,

wherein the T cells are maintained under conditions that allow for expansion, and

wherein the solid supports are removed from contact with the T cells after a time period of less than 120 hours.

Clause 103. The method of clause 102, wherein the first agent is an anti-CD3 antibody.

Clause 104. The method of clause 102 or 103, wherein the second agent is an antibody.

Clause 105. The method of clause 102,103 or 104, wherein the T cells are contacted with a third agent which is an antibody or a non-antibody protein.

Clause 106. The method of clause 105, the non-antibody protein is a chemokine or cytokine.

1. A method for selectively expanding members of a T cell subpopulation, the method comprising exposing a mixed population of T cells to:
(a) a first agent which provides a primary activation signal to the members of the T cell subpopulation, thereby activating the T cells, and
(b) a second agent and a third agent, each of which stimulates two or more different accessory molecules on the members of the T cell subpopulation, thereby stimulating the proliferation of the activated T cells of (a),
wherein the ratios of the first agent, the second agent, and the third agent are adjusted to induce the members the T cell subpopulation to selectively expand over members of other T cell subpopulations.

2. The method of claim 1, wherein the first agent is an anti-CD3 antibody.

3. The method of claim 1, wherein the second agent is an antibody.

4. The method of claim 1, wherein the third agent is an antibody or a non-antibody protein.

5.-6. (canceled)

7. The method of claim 1, wherein the ratio of the first agent, the second agent, and the third agent are adjusted such that the first agent is lower in concentration compared to the second or third agent.

8. The method of claim 7, wherein the lower concentration of the first agent is about 0.34 units.

9. The method of claim 8, wherein the first agent is an anti-CD3 antibody at a concentration of about 0.34 units, and a second agent is an anti-CD28 antibody at a concentration of 3.4 units.

10.-12. (canceled)

13. The method of claim 12, wherein the T cell subpopulation selectively expanded are memory T cells.

14.-68. (canceled)

69. A method for selectively altering the proportional ratio of two T cell subtypes in a sample, the method comprising contacting a sample comprising a mixed population of T cells with at least two stimulatory agents, wherein the stimulatory agents provide different amounts of signals to the T cells in the mixed population, wherein one T cell subtype selectively expands as compared to a second T cell subtype.

70. The method of claim 69, wherein the sample comprises buffy coat cells derived from an individual.

71. The method of claim 69, wherein the at least two stimulatory signals stimulate CD3 and CD28 receptors.

72. The method of claim 69, wherein at least one T cell subtype is selectively eliminated from the mixed population.

73. The method of claim 69, wherein Treg T cells are increased in proportion with respect to all T cells within the mixed population.

74. The method of claim 69, wherein the total number of memory T cells is decreased in the sample.

75. The method of claim 69, wherein the amount of stimulatory signal CD3 is less than half than the CD28 stimulatory signal.

76.-101. (canceled)

102. A method for the activation and expansion of T cells, the method comprising contacting a mixed population of T cells with:
(a) a first agent which provides a primary activation signal to the members of a T cell subpopulation by stimulating a molecule on the members of the T cell subpopulation, and
(b) a second agent that stimulates a molecule on the members of the T cell subpopulation that is different than the molecule stimulated by the first agent,
whereby T cells in the population are activated and expand,
wherein the first agent and the second agent are bound to one or more solid supports,
wherein the T cells are maintained under conditions that allow for expansion, and
wherein the solid supports are removed from contact with the T cells after a time period of less than 120 hours.

103. The method of claim 102, wherein the first agent is an anti-CD3 antibody.

104. The method of claim 102, wherein the second agent is an antibody.

105. The method of claim 102, wherein the T cells are contacted with a third agent is an antibody or a non-antibody protein.

106. (canceled)
